(19) United States
(12) Patent Application Publication
(10) Pub. No.: US 2024/0182842 A1
(43) Pub. Date: Jun. 6, 2024

(54) TEMPERATURE CONTROL DEVICES, TEMPERATURE-RESPONSIVE PROTEINS, AND METHODS OF USING THE SAME

(71) Applicant: THE TRUSTEES OF THE UNIVERSITY OF PENNSYLVANIA, Philadelphia, PA (US)

(72) Inventors: Lukasz Bugaj, Philadelphia, PA (US); Brian Chow, Cherry Hill, NJ (US); William Benman, Philadelphia, PA (US); Zikang Huang, Philadelphia, PA (US)

(21) Appl. No.: 18/527,020

(22) Filed: Dec. 1, 2023

Related U.S. Application Data
(60) Provisional application No. 63/385,680, filed on Dec. 1, 2022.

(51) Int. Cl. C12M 1/34; C12M 1/32

(52) U.S. Cl. CPC C12M 41/12; C12M 23/12

(57) ABSTRACT

Provided herein are a device for well plate temperature control, a method of independently controlling the temperature in individual wells of a microwell plate, and a temperature-responsive protein. The device includes a microwell plate with at least one well formed therein; a temperature control assembly including a printed circuit board with at least one pair of thermistors extending therefrom, each of the at least one pair of thermistors arranged and disposed to align with one of the at least one wells; and a microcontroller configured to individually control each of the at least one pair of thermistors. The method includes positioning the temperature control assembly adjacent to the well plate such that each pair of thermistors extends into one of the wells, and independently providing a current flow to each of the at least one pair of thermistors to separate heat each well. The temperature-responsive protein includes a BcLOV4 protein variant having a point mutation at Q355, the variant having at least 80% sequence homology with the wild-type BcLOV4 protein.

### CROSS-REFERENCE TO RELATED APPLICATIONS

The present application claims priority under 35 U.S.C. § 119(e) to U.S. Provisional Patent Application No. 63/385,680, filed Dec. 1, 2022, which application is incorporated herein by reference in its entirety.

### STATEMENT REGARDING FEDERALLY SPONSORED RESEARCH OR DEVELOPMENT

This invention was made with government support under GM138211 awarded by the National Institutes of Health and 2145699 awarded by the National Science Foundation. The government has certain rights in the invention.

### BACKGROUND OF THE INVENTION

There are many situations where one would want to control proteins and cells remotely and on-demand. One attempt at achieving this includes blue light-activatable control of cells. However, blue light does not penetrate tissues efficiently (˜1 mm). Another approach includes the use of heat shock promoters, which induce transcription in response to heat. However, heat shock promoters also respond to other cell stresses and thus this system could be inadvertently triggered. Moreover, heat shock promoters also drive other, endogenous cellular programs, so thermal induction would be at least somewhat non-specific. These promoters may also require extended heating to generate strong output (15 min-1 hr), which can damage surrounding tissue. Furthermore, although heat shock promoters also only allow transcriptional control, there are no ways to use temperature for post-translational control (e.g., for signaling, for proteolysis, other post-transloational modifications, etc.)

Accordingly, there remains a need in the art for articles and methods that improve on existing remote control of proteins and cells by providing focused thermal control. The present invention addresses this need.

### SUMMARY OF THE INVENTION

In one aspect a device for well plate temperature control includes a microwell plate comprising a top well plate surface, a bottom well plate surface, and at least one well extending from the top well plate surface towards the bottom well plate surface; a temperature control assembly comprising a printed circuit board having a top circuit board surface and a bottom circuit board surface, and at least one pair of thermistors extending outwardly from the bottom circuit board surface, each of the at least one pair of thermistors arranged and disposed to align with one of the at least one wells when the bottom circuit board surface is positioned adjacent to the top well plate surface; and a microcontroller configured to individually control each of the at least one pair of thermistors.

In some embodiments, each pair of thermistors comprises a heating thermistor and a measurement thermistor. In some embodiments, the heating thermistor comprises a resistive heating thermistor. In some embodiments, the temperature control assembly further comprises at least one shift register and at least one control transistor. In some embodiments, the microcontroller communicates with the at least one shift register to control the at least one control transistor, and the at least one control transistor determines the current flow through each of the thermistors. In some embodiments, the temperature control assembly further comprises at least one voltage divider and at least one multiplexer in electrical communication with the microcontroller. In some embodiments, the temperature of each of the at least one wells is measured through the at least one voltage divider and the at least one multiplexer. In some embodiments, the at least one well comprises 96 wells.

In some embodiments, each of the at least one pair of resistors includes a liquid-tight covering. In some embodiments, the liquid-tight covering comprises heatshrink tubing. In some embodiments, the liquid-tight covering further comprises a conformal coating over the heatshrink tubing. In some embodiments, the conformal coating comprises a silicone conformal coating. In some embodiments, each of the resistors independently includes the liquid-tight covering. In some embodiments, the device further includes an individual feedback loop between the microcontroller and each of the at least one pair of thermistors. In some embodiments, the device further includes an adapter positioned between the microwell plate and the temperature control assembly, the adapter being arranged and disposed to position the temperature control assembly relative to the well plate.

In another aspect a method of independently controlling the temperature in individual wells of a microwell plate includes providing the device according to one or more of the embodiments disclosed herein, filling one or more of the at least one wells with a liquid sample, positioning the bottom circuit board surface adjacent to the top well plate surface, the positioning immersing the at least one pair of thermistors in the one or more wells with the liquid sample, independently providing a current flow to each of the at least one pair of thermistors, the current flow to each of the at least one pair of thermistors providing a desired temperature in the corresponding well through resistive heating. In some embodiments, the further comprises an adapter positioned between the microwell plate and the temperature control assembly, the adapter being arranged and disposed to position the temperature control assembly relative to the well plate.

In another aspect a temperature-responsive protein includes a BcLOV4 protein variant having a point mutation at Q355, wherein the variant includes at least 80% sequence homology with the wild-type BcLOV4 protein. In some embodiments, the Q355 point mutation is Q355N. In some embodiments, the protein further includes a point mutation at C292. In some embodiments, the C292 point mutation is selected from the group consisting of C292A, C292R, C292N, C292D, C292E, C292Q, C292G, C292H, C292I, C292L, C292K, C292M, C292F, C292P, C292S, C292T, C292W, C292Y, C292V. In some embodiments, the C292 point mutation is C292A. In some embodiments, the C292 point mutation is selected from the group consisting of C292R, C292N, C292D, C292E, C292Q, C292G, C292H, C292I, C292L, C292K, C292M, C292F, C292P, C292S, C292T, C292W, C292Y, C292V. In some embodiments, the point mutation further comprises deletion of one or more of amino acids 1-97.

In another aspect, a method of controlling the membrane localization of a protein includes providing the protein according to one or more of the embodiments disclosed herein and exposing the protein to a temperature above or below an activation temperature. In some embodiments, the C292 point mutation is selected from the group consisting of C292A, C292R, C292N, C292D, C292E, C292Q, C292G, C292H, C292I, C292L, C292K, C292M, C292F, C292P, C292S, C292T, C292W, C292Y, C292V.

### BRIEF DESCRIPTION OF THE DRAWINGS

FIG. 1 shows a photograph of a temperature control device.

FIG. 2 shows a photograph of a temperature control assembly.

FIG. 3 shows a photograph of a temperature control assembly with an adapter.

FIG. 4 shows a schematic of a temperature control assembly.

FIG. 5 shows graphs illustrating sample heating of 3 wells through 15° C. 3 of the 96 wells were heated to 40 degrees and then allowed to recover to ambient temperature. Traces show precise, rapid attainment of the target temperature and subsequent return to baseline. Heating dynamics show a high level of precision, accuracy and low variability between wells.

FIGS. 6A-G show images and graphs illustrating that single-component BcLoV4 fusions allow control of Ras and PI3K signaling. (A) BcLOV4 binds the cell membrane when exposed to blue light. The three BcLOV4 domains represent the LOV, DUF and RGS domains, as previously described15. (B) Light-induced membrane recruitment of BcLOV4 fused to the SOS_{cat} catalytic domain will induce Ras/Erk signaling. (C) Analogous recruitment of the iSH domain will induce PI3K/Akt signaling. (D-E) Five minutes of blue-light stimulation (160 mW cm−2 at 20% duty cycle) increases intracellular ppErk levels in cells that express BcLOV-SOS_{cat} (D) and increases pAkt in cells that express BcLOV-iSH (E). a.u., arbitrary units. Gray zone indicates the change in ppErk or pAkt in wild-type cells that were stimulated with 10% calf serum for 10 min. Data represent means of three biologically independent replicates, each representing the mean signal intensity from approximately 2,000 to 4,000 single cells. (F-G) Light intensity dose-response of (F) ppErk fold-change induction in BcLOV-SOS_{cat}-expressing cells or (G) pAkt fold-change induction in BcLOV-iSH cells at 100% duty cycle after 5 min of illumination. Data represent means±s.d. of three biologically independent replicates, each representing the mean signal intensity from approximately 300 to 500 single cells. All stimulation in A-G was achieved using the optoPlate-96. All stimulation and environmental conditions for all figures can be found in Supplementary Table 1.

FIGS. 7A-H show images and graphs illustrating that BcLoV-induced signaling dynamics depend on temperature and light exposure. (A) Schematic of experimental protocol. (B) Sustained stimulation of BcLOV-SOS_{cat} (160 mW cm-2 at 20% duty cycle) reveals that the ppErk signal decays rapidly after an initial increase, whereas activation by iLID-SOS_{cat} remains sustained. (C) Recovery (dark) periods of up to 3 h after signal decay do not permit recovery of activatable BcLOV-SOS_{cat}, suggesting that BcLOV4 inactivation is effectively irreversible. (D) BcLOV-SOS_{cat} signal dynamics were examined under variable light and temperature conditions. (E) Schematic for how the optoPlate-96 was repurposed to allow independent control of experimental light and temperature conditions. For more information, see FIGS. 20A-G. (F) Steady-state sample temperature was a linear function of the intensity of the 72 heater LEDs (precise intensity-temperature relationship should be determined empirically for each individual optoPlate). See Methods. (G) At a given light exposure level (here, 160 mW cm−2 at 20% duty cycle), BcLOV-SOS_{cat} signal decays more rapidly at higher temperatures. (H) At a given temperature (here, 36° C.), BcLOV-SOS_{cat} decay increases with increased light exposure (variable duty cycles of 160 mW cm−2 light). Data points in (B), (C), (G), and (H) represent the mean±s.e.m. of ˜1,000-4,000 individual cells. Traces in (G) and (H) are exponential decay functions fit to data points at each temperature and duty cycle, as described in the Methods section. Data are normalized between the minimum and maximum of each trace. Normalization was performed separately for each temperature and duty cycle to highlight the change in the rate of BcLOV-SOS_{cat} inactivation rather than absolute signal. Absolute signal traces can be found in FIGS. 21A-E. a.u., arbitrary units.

FIGS. 8A-G show images and graphs illustrating that BcLoV4 membrane translocation dynamics depend on temperature and light exposure. (A) BcLOV-mCherry membrane recruitment was quantified at various temperatures and light exposures using live-cell imaging. (B) Representative images of membrane recruitment at low and high temperatures. Activation at 25° C. permitted sustained membrane recruitment, whereas recruitment at 37° C. was transient (stimulation performed at 1.45 W cm−2 and 3% duty cycle). Image brightness was adjusted at each time point for clarity to account for photobleaching. (C) Quantification of membrane recruitment at various temperatures (1.45 W cm−2 at 3% duty cycle) reveals a temperature-dependent decay of membrane translocation. (D) Quantification of membrane recruitment at various light exposures (at 36° C. and 1.45 W cm−2) shows light-dependent decay of BcLOV-mCherry translocation. Each trace is the mean membrane fluorescence±s.e.m. of three biologically independent samples, with each replicate representing the mean of ˜100 cells. See FIGS. 23A-G for unnormalized traces and quantification workflow. (E) Schematic of a three-state model of BcLOV4 membrane translocation. (F) Fitting the model to live-cell translocation data provides parameter values for k₁, k₂ and k₃ (T). (G) Heatmap depicts the decay rate of BcLOV4 membrane localization as a function of temperature and light exposure. Decay rates were calculated by simulating sustained illumination over a range of duty cycles and temperatures and fitting the modeled decay rate to a single exponential decay. Color indicates the decay constant λ (1 divided by the time to reach 37% of maximum signal). Larger λ indicates faster decay. See FIGS. 23A-G and 26 and Methods for imaging and model details.

FIGS. 9A-D show images and graphs illustrating that modeling predicts BcLoV-SoS_{cat}-induced ppErk dynamics and reveals dynamic filtering properties of Ras/Erk signaling. (A) A model of ppErk activation was developed by integrating the BcLOV4 membrane translocation model with a transfer function model that describes the input/output response of SOS_{cat} membrane localization (input) to ppErk activation (output). (B) Filtering properties of a 1° versus 2° LPF. 1° LPFs attenuate high-frequency inputs less than 2° LPFs. (C) A 1° LPF with 2-mHz cutoff frequency best describes ppErk dynamics when stimulated with fast 2 min ON/2 min OFF BcLOV-SOS_{cat} oscillations. Data points are the mean±s.d. of three biologically independent replicates, with each replicate representing the mean of ˜1,000-2,000 cells. (D) Heatmap depicts the predicted Erk activation dynamics resulting from BcLOV4 membrane translocation dynamics over the indicated light and temperature conditions. Plots show model predictions of Erk activation at the indicated experimental conditions, and data points show experimental results. Data points represent means±s.e.m. of ˜1,000-4,000 cells. Unnormalized plots are presented in FIGS. 27A-E. See Methods for model details.

FIGS. 10A-H show images and graphs illustrating BcLoV4 and BcLoV-SoS_{cat} in zebrafish embryos and Drosophila cells. (A) Blue-light-induced membrane translocation of BcLOV-mCherry in a zebrafish embryo (24 h post fertilization (hpf)). Scale bar, 50 μm; inset scale bar, 20 μm. (B) BcLOV-mCherry translocation is sustained over 90 min in zebrafish embryos. Data represent mean±s.d. of ten cells. (C) Schematic of ErkKTR activity. ErkKTR is nuclear when Erk signaling is off and translocates to the cytoplasm when Erk is activated. (D) The Ras/Erk pathway can be reversibly stimulated over multiple cycles in zebrafish embryos (24 hpf) that coexpress BcLOV-SOS_{cat} and ErkKTR-BFP, as measured by ErkKTR-BFP translocation. White arrows highlight nuclei where ErkKTR translocation is evident. Scale bar, 10 μm. (E) Sustained illumination of BcLOV-SOS_{cat} permits sustained elevated Erk activity in 24 hpf zebrafish embryos. Plot shows ErkKTR cytoplasmic/nuclear ratios of 12 single cells (light gray; blue trace represents mean) measured over two experiments. Trajectories are normalized between 0 and 1 to permit comparison between experiments. For (A)-(E) stimulation was performed using 1.45 W cm−2 488 nm light at 1.5% duty cycle. (F) BcLOV-mCherry membrane translocation in Drosophila S2 cells stimulated with blue light (1.45 W cm−2 at 3% duty cycle) for 90 min. Scale bar, 10 μm. (G) Quantification of (F) shows sustained membrane translocation in S2 cells. Data represent mean±s.d. of ten cells. (H) Sustained stimulation of BcLOV-SOS_{cat} in S2 cells shows sustained elevated ppErk levels over 90 min, measured by immunofluorescence. Data represent the mean±s.d. of three biologically independent samples, with each replicate representing the mean of ˜100-200 cells. Stimulation was performed at (160 mW cm−2 at 20% duty cycle). All experiments were performed at room temperature. a.u., arbitrary units.

FIGS. 11A-E show images and graphs illustrating that BcLoV4 temperature sensitivity enables orthogonal multiplexing of multiple blue-light sensitive tools in single cells. (A) Schematic of how coexpression of BcLOV4 and iLID/sspB can allow blue-light control of three separate cell states. (B) Coexpression in of BcLOV-mCherry and iLID/ sspB-GFP in HEK 293 T cells demonstrates three-state control using blue light with or without temperature inactivation. Light stimulation was performed at 37° C. using 1 s of blue light (1.45 W cm−2) every 30 s for 10 min in the presence or absence of prior BcLOV inactivation. Prior inactivation was achieved by illuminating with these same light settings for 1 h. (C) Quantification of light-induced membrane binding (activation) of BcLOV4 and iLID in the absence (left) or presence (right) of prior heat inactivation. Traces are the normalized mean±s.e.m. of three biologically independent replicates, with each replicate representing the mean of ˜100 cells. (D) Schematic of how coexpression of BcLOV4 and Cry2 can allow blue-light control of four separate cell states. (E) Coexpression of BcLOV-GFP and Cry2-mCherry in HEK 293 T cells demonstrates four-state control. ‘Short light’ (10 min) and ‘long light’ (45 min) exposure were both achieved using 100 ms of light (1.45 W cm−2) every 30 s at 30° C. To achieve the Cry2-ONLY state (bottom row), images were acquired after BcLOV was inactivated using 1 s of blue light (1.45 W cm−2) every 30 s for 45 min at 37° C. Scale bar, 10 μM. See Methods and Supplementary Table 1 for full illumination conditions. Time is given in minutes. The multiplexing experiments depicted are representative of two independent experiments for each pair of optogenetic proteins.

FIG. 12 shows images illustrating modulation of strength of BcLOV-T membrane association with polybasic domains.

FIG. 13 shows images illustrating modulation of temperature switchpoint of BcLOV-T membrane association.

FIGS. 14A-C show representative images of immunofluorescence staining of ppErk in BcLOV-SOS_{cat} and WT cells. (A-C) Immunofluorescent ppErk signal from unilluminated WT (A) vs unilluminated (B) and illuminated (C) NIH 3T3s expressing BcLOV-SOS_{cat}. Illuminated cells show increase in fluorescence compared to unilluminated cells. Cells are stained for phospho-Erk, with an Alexa488-conjugated secondary antibody.

FIG. 15 shows time course of early ppErk activation in BcLOV-SOS_{cat} cells upon illumination. Early activation of Erk using BcLOV-SOS_{cat}. NIH 3T3 cells expressing BcLOV-SOS_{cat} were exposed to blue light for the time shown, fixed, and stained for ppErk in order to establish the activation kinetics of Erk activation by SOS, with 30 s resolution. The peak of signaling occurs ˜4.5 minutes. Data points represent the mean+/−1 SD of three biologically independent replicates. Each replicate mean represents ˜1000 cells.

FIG. 16 shows unnormalized traces of ppErk induction presented in FIG. 7B. ppErk levels observed through immunofluorescence in response to BcLOV-SOS_{cat} vs iLid-SOS_{cat} stimulation (160 mW/cm2 at 20% duty cycle). Data points represent mean+/−SEM of ˜1000-4000 single cells.

FIG. 17 shows a schematic of iLid-based probes of Ras/Erk and PI3K/Akt signaling. A) The iLid protein heterodimerizes with sspB when exposed to blue light. Anchoring iLid to the cell membrane allows for light inducible membrane recruitment. B) Fusing sspB to the iSH domain allows for light inducible stimulation of PI3K signaling. C) Fusing sspB to the SOScat domain allows for light inducible stimulation of Ras/Erk signaling. D) Schematic of iLid expression construct.

FIGS. 18A-B show PI3K signal decay in BcLOV-iSH cells despite constant illumination. (A) pAkt activation through stimulation of BcLOV-iSH and iLid-iSH over 60 min (160 mW/cm2 at 20% duty cycle) demonstrates sustained signal induction by iLid-iSH but transient signal induction by BcLOV-iSH. (B) Unnormalized data presented in (A). Data points represent mean+/−SEM of ˜2000-3000 single cells.

FIG. 19 shows mild recovery of BcLOV-SOScat is at least partially due to new protein synthesis. BcLOV-SOS_{cat} cells were stimulated with blue light for 45 minutes with and without translation inhibitor cycloheximide (CHX, 20 μg/mL) at 37° C. Cells were then allowed to recover in the dark for 3 hours and were subsequently restimulated. The modest signal recovery of BcLOV-SOS_{cat} was further attenuated in the presence of CHX, suggesting that signal recovery is at least partially due to protein synthesis. Data represent mean +/−1 SEM of ˜1000-2000 cells measured for each condition. Where error bars are not visible, they are smaller than the data point.

FIGS. 20A-G show repurposing the optoPlate-96 for dual thermal and optical control of cells. (A) Sample heating across a microwell plate was characterized with a custom-built 12-channel temperature probe. (B) Temperatures of sample wells were measured at the indicated positions (colored circles) to verify the uniformity of sample heating across the sample plate. (C) To test sample heating as a function of duty cycle, 3 simultaneous 8-point time courses were simulated (160 mW/cm2) from 0-60 min, and heating was measured in the wells (filled with PBS) that would have received the indicated duration of light. The three time-series were performed at 1%, 5%, and 20% duty cycle (500 ms light every 50 s, 10 s, or 2.5 s). (D) Temperature remained stable over the course of the experiment, with a ˜0.5° C. increase in the 60′ well of the 20% duty cycle condition at the end of the experiment. These results suggest that 20% duty cycle is the most intense stimulation we can perform while avoiding heating in this experimental arrangement. Other experimental parameters (lower light intensity, taller adapter, fewer LEDs) would permit a higher duty cycle with less or no heating. (E-F) Uniformity of sample heating was assessed by measuring sample well temperature under varying illumination intensities of the heating LEDs (constant light). We observed uniform temperature increases across the plate as a function of heater LED intensity (E and F). (G) Experiments in (E-F) were repeated across heater intensity settings from 0-3000, and plateau temperature in each of the 12 wells was plotted for each intensity setting. Black dot represents the mean temperature from each of the 12 wells, whose temperatures are individually depicted by the colored data points. Results reveal a linear relationship between heater LED intensity and plateau temperature of the sample wells. Heater LEDs allow programmatic increase of temperature above ambient. All characterizations were were performed at room temperature. See FIG. 7E for analogous characterization performed in a cell culture incubator that was set to 32° C., allowing linear control of temperature between 32-42° C.

FIGS. 21A-E show unnormalized BcLOV-SOS traces from FIG. 2, BcLOV-iSH, and iLid probes at various temperatures. (A) Unnormalized ppErk levels resulting from BcLOVSOS_{cat} stimulation (160 mW/cm2 at 20% duty cycle) at various temperatures. (B) NIH 3T3 cells expressing BcLOV-iSH were stimulated with blue light at 30° C. or 37° C. Similar to BcLOV-SOS_{cat}, signaling was transient at 37° C. but was stable at 30 ° ° C. Traces were normalized at each temperature independently. (C-D) NIH 3T3 cells expressing either iLid-SOS_{cat} (C) or iLid-ISH (D) were stimulated with blue light at 30° C. or 37° C. In contrast to BcLOV4-induced signaling, iLid drove sustained levels of ppErk or pAkt at both low and high temperatures. Traces were normalized at each temperature independently. (E) Unnormalized ppErk levels resulting from BcLOV-SOScat stimulation at various duty cycles. (36° C. and stimulated at 160 mW/cm2). Data points in A/E represent the mean+/−SEM of ˜1000-3000 cells. Data points in B/C/D represent the mean+/−1 SD of three biologically independent replicates. Each replicate mean represents ˜2000-3000 cells.

FIGS. 22A-D show reversibility of signaling induced by BcLOV-SOScat. (A) Schematic of ErkKTR activity. ErkKTR is nuclear when Erk signaling is off and translocates to the cytoplasm when Erk is activated. (B) Cells expressing BcLOV-SOScat and ErkKTR-mCh were stimulated with repeating cycles of 10 minutes of blue light followed by 20 minutes of darkness. (C-D) ErkKTR imaging shows that after 10 minutes of blue light stimulation, ErkKTR has translocated out of the nucleus, and after 20 minutes of darkness, translocates back into the nucleus. This process was repeated 3 times to demonstrate the reversibility of Erk signaling using BcLOV-SOScat at (C) 30° C. and (D) 37° C.

FIGS. 23A-G show BcLOV quantification workflow and unnormalized fold-change of BcLOV-mCherry translocation presented in FIGS. 8A-G. (A) HEK 293T cells were transfected with BcLOV-mCh and an iRFP-CAAX membrane marker. Cells were stimulated with blue light. A small number (˜2%) of iRFP-CAAX images were chosen at random to train the machine learning image processing software iLastik to identify iRFP-CAAX enriched cell membranes. iLastik produced masks of iRFP-CAAX membrane which were then multiplied by BcLOV-mCh images to quantify mCherry present at the membrane. The raw intensity of mCherry was then used to normalize all images for bleaching and cell number, normalized membrane intensity was normalized between 0 and 1 for each time series, and means of biological triplicate time series were calculated. Finally, a moving mean average filter was applied to smooth the high frequency noise associated with raw imaging data. (B-G) Quantification of live cell imaging of BcLOV-mCh membrane recruitment at (B) 26.4° C., (C) 29.4° C., (D) 31.8° C., (E) 34.4° C., (F) 36.6° C., (G) 39.3° C. when exposed to 1.45 W/cm2 at 3% duty cycle. Each trace is the mean+/−SEM of three biologically independent samples, each representing the mean of ˜100 cells.

FIGS. 24A-C show temperature-dependent BcLOV4 dynamics are not due to protein degradation. (A) BcLOV4 was illuminated for 1 s every 30 s at 25 and 37° ° C. After 45 minutes, total mCherry intensity had decreased by ˜15% at both temperatures, likely due to bleaching. (B) In contrast, membrane recruitment of BcLOV4 is more transient at 37° C. compared to 25° C., demonstrating that loss of membrane fluorescence does not result from BcLOV-mCh degradation. Data in (B) is reproduced from FIGS. 23A-G. Each trace is the mean+/−SEM of three biologically independent replicates. Each replicate the mean measurement of ˜100 cells. (C) BcLOV-mCh was stimulated in HEK 293T cells in the presence or absence of proteosome inhibitor MG132 (10 μM). Proteosome inhibition did not prevent membrane dissociation, indicating that membrane dissociation does not require protein degradation. Traces represent the mean membrane localization of ˜100 cells

FIGS. 25A-B show inactivation of BcLOV-mCh translocation is effectively irreversible. (A) HEK 293T cells expressing BcLOV-mCh were either stimulated for 10 minutes (“WT BcLOV”), inactivated by exposure to blue light for 1 hour (37° C.) and then stimulated for 10 minutes (“Inactivated”), or were inactivated and stimulated as above but in the presence of translation inhibitor cycloheximide (“Inactivated +CHX”, 20 μg/mL CHX). Cells were allowed to recover for 3 hrs after inactivation, prior to the 10 minute stimulation. (B) Quantitation of membrane translocation resulting from conditions in (A), as measured through confocal microscopy. WT BcLOV-mCh showed strong membrane recruitment, whereas inactivated BcLOV-mCh exhibited only slight recruitment after 3 hrs recovery. This low-level recruitment was further abolished in the presence of CHX, suggesting that new protein synthesis accounts in part for recovery of BcLOV4 function after inactivation. Data points are the means+/−SEM of three biologically independent replicates, and each replicate represents the mean of ˜100-200 cells. Ribbons represent standard error.

FIG. 26 shows values of k3 obtained experimentally show an exponential dependence on temperature. Values for k3 were found for each temperature tested for BcLOVmCh membrane recruitment (FIGS. 23A-G) and were fit to an exponential curve. This allowed for extrapolation of the value of k3 at any temperature within this range. See Methods for complete model details.

FIGS. 27A-E show unnormalized ppErk stimulation in BcLOV-SOScat cells at various temperatures and duty cycles as presented in FIGS. 9A-D. Data points in all plots above represent the mean +/−SEM of ˜1000 single cells.

FIG. 28 shows maximum intensity light used in this study was not toxic to cells. A) NIH 3T3 cells expressing BcLOV-SOScat were exposed to the maximum light intensity used in this study (160 mW/cm2 at 50% duty cycle) and stained with Nucview488, a fluorescent marker of caspase-3 activation. Cells were exposed from 0-4 hrs of blue light, and 20 μM staurosporine was used as a positive control of caspase activation and cell death. No measurable increases in caspase activation were observed under any light condition. Each density plot represents Nucview intensities of ˜1000-2000 cells.

FIG. 29 shows representative gating strategy for cell sorting. WT cells (A) were analyzed to establish the lower edge of gating. The lower bound was set to exclude >99.9% of WT cells . BcLOV-BFP-SOS displayed a rightward shift in fluorescence, allowing ˜1% of the population to be accepted.

FIGS. 30A-H show images and schematics illustrating harnessing BcLOV4 thermosensitivity to generate a purely temperature-inducible protein. (A-C) BcLOV4: reversible translocation in response to light to temperature. (A) Schematic of BcLOV4, a naturally light-and temperature-responsive protein. BcLOV4 translocates to the membrane under blue light and reverts to the cytoplasm in the dark. From the membrane-bound (lit) state, elevated temperatures induce dissociation from the membrane, and lower temperatures induce reassociation. (B) Representative images showing translocation to the membrane when exposed to blue light in HEK 293T cells. Scale bar represents 15 μm. (C) Extended illumination at elevated temperatures (2 hr at 37° C., left) causes subsequent disassociation from the membrane, but reversion to lower temperatures (25° C., right) allows reassociation with the membrane. (D-H) Melt (membrane localization with temperature): a purely thermoresponsive protein. (D) Schematic of Melt. Melt is BcLOV4 with a Q355N mutation, which mimics the lit state of BcLOV4. (E) Representative images showing that Melt is cytoplasmic at 37° C. and does not translocate to the membrane upon light stimulation, unlike BcLOV4. However, Melt retains temperature sensitivity and translocates to the membrane upon lowering temperature to 25° C. (F). Scale bar=15 μm. (G-H) Comparison of optical (G) and thermal (H) responses of wt BcLOV and Melt. See FIG. 35 for details on quantification. Data represent mean+/−1 SEM of ˜100 cells.

FIGS. 31A-K show graphs and images illustrating characterization of Melt membrane association using the thermoPlate. (A) Image of the thermoPlate, a device for thermal control of individual wells in 96-well plate format. (B) The thermoPlate consists of 96 pairs of thermistors, which serve as temperature readers (R) and heaters (H). (C) Schematic of an H/R pair inserted in the well of a 96 well plate. Simultaneous heating and reading of temperature allows PID feedback-controlled heating. (D) Heating of 16 individual wells in a 96-well plate with <1° C. resolution over 16 hours. Each trace represents the temperature in a single well as recorded by the (R) thermistor in each well. Scale bar represents 20 μm. (E) thermoPlate heating of HEK 293T cells stably expressing Melt allowed measurement of steady-state membrane association (14 hr of heating). Data points represent mean+/−1 SD of three wells, each containing ˜200 imaged cells. (F) Rapid heating and cooling kinetics enabled by the thermoPlate. Trace shows a single well heated to 37° ° C. for 30 min and subsequent return to 27° C. A 10° C. change in setpoint temperature is achieved in ˜2.5 and 6 min for heating and cooling, respectively. (G) Representative images of live-cell images showing Melt membrane binding over multiple cycles of 1 hr at 37° C. followed by 3 hr at 27° C. Scale bar=10 μm. (H) Plot of membrane bound Melt while undergoing cycles of 30 min at 37° C. followed by 5 hr at 27° C. Traces represent mean+/−1 SEM of ˜100 cells. (I) Kinetics of Melt membrane dissociation when exposed to various temperatures after 24 hr of culture at 27° C. (J) Kinetics of Melt membrane reassociation at 27° ° C. after prior exposure to 6 hrs of the indicated temperatures. (K) Kinetics of Melt membrane reassociation at 27° C. after prior exposure to 37° C for the indicated durations. Each trace in (I-K) represents the mean+/−1 SEM of ˜1000 cells. Data were collected from HEK 293T cells that stably expressed Melt-mCh. For H, I, J, and K membrane binding was normalized to the first time point of each condition.

FIGS. 32A-H show graphs and images illustrating thermal control over diverse intracellular processes using Melt. (A-F) Temp-induced signaling (Ras or EGFR). (A) Schematic of thermal control of Ras-Erk signaling by membrane recruitment of the SOS2 catalytic domain (meltSOS). (B) Thermal activation and inactivation of Ras as assayed by immunofluorescence for activation of the downstream Erk kinase (phospho-Erk, or ppErk). Data points represent the mean+/−1 SEM of ˜500 cells. (C) Representative images of ppErk immunofluorescence from meltSOS-expressing cells cultured at the indicated temperatures for 24 hours, 1 hour, and 1 hour respectively. Scale bars represent 40 μm. (D) Schematic of thermal control of EGFR receptor signaling by membrane recruitment and clustering of the EGFR intracellular domain (meltEGFR). (E) Thermal activation and inactivation of EGFR, assayed through immunofluorescence for ppErk. Each data point represents the mean+/−1 SEM of ˜500 cells. (F) Representative images of ppErk immunofluorescence from meltEGFR cells cultured at the indicated temperatures for 24 hours, 1 hour, and 1 hour respectively. Scale bars represent 40um. (G-I) Temp-induced proteolysis. (G) Schematic of thermal control of proteolysis with meltTEVp. At low temperatures, meltTEVp translocates to the membrane where it cleaves a membrane-bound fluorescent reporter of proteolysis (FlipGFP). (H) Representative images of

FlipGFP fluorescence in cells expressing meltTEVp or TEVp cultured at 37° C. or 27° ° C. for 24 hr. Scale bars=20 μm. (I) Quantification of FlipGFP fluorescence in cells expressing either meltTEVp or TEVp cultured at the indicated temperature for 24 hours. Each bar represents the mean+/−1 SEM of ˜1000 cells, normalized between negative and positive controls at each temperature (see FIGS. 36A-C for normalization process). (J-L) Temp-induced nuclear localization. (J) Schematic of thermal control of nuclear translocation with meltNLS/NES. (K) Quantification of nuclear localization meltNLS/NES and Melt-mCh exposed to cycles of 37° C. and 27° C. Traces represent the mean+/−1 SEM 576 of ˜1000 cells. See Example 3—Methods for details on quantification of nuclear localization. (L) Representative images of nuclear localization of meltNLS/NES and Melt-mCh at the temperatures/timepoints found in (K). Scale bar represents 10 μm.

FIGS. 33A-R show graphs and images illustrating tuning of Melt membrane binding and thermal switch-point allows application of Melt-based tools in mammalian temperature ranges. (A-H) Amplitude and critical temperature tuning. (A) Tuning the amplitude of Melt membrane association. (B) Polybasic (PB) domains from the STIM or Rit proteins were fused to Melt to test their ability to increase Melt membrane binding strength. (C) Representative images showing stronger membrane binding (higher membrane/cyto ratio) of Melt fused to PBs compared to Melt alone. Melt constructs were stably expressed in HEK 293T cells and are shown after 24 hrs of culture at 27° C. and after subsequent heating to 37° C. for 6 hrs. Scale bar=20 μm. (D) Quantification of steady-state membrane association of Melt-PB fusions after culture at indicated temperatures for 12 hours. Data represent mean+/−1 SD of three wells with ˜200 cells quantified per well. (E) Tuning Melt switch-point temperature for use within temperature ranges relevant for mammals, between 37° C. and 42° C. (F) Schematic of Melt with a C292A mutation with and without STIM PB domain. (G) Representative images of membrane localization of Melt, Melt+C292A, or Melt+C292A+STIM fusion at 35° C for 24 hours and subsequent culture at 41° C. for 6 hours. Scale bar=20 μm. (H) Quantification of steady-state membrane binding (14 hrs) of Melt variants between 27 and 42° C. Data represent mean+/−1 SD of three wells with ˜500 cells quantified per well. Data are normalized between min and max values for each construct. Unnormalized traces can be found in FIGS. 33D and 39. (I-L) Temp-regulated EGFR signaling (above 37° C.). (I) Thermal control of EGFR at and above 37° ° C. using Melt-37. (J)

Immunofluorescence quantification of pathway activation in HEK 293T cells stably expressing meltEGFR-37. Cells were incubated at indicated temperatures for 75 min before fixation. Bars represent mean+/−1 SD of three wells with ˜1000 cells quantified per well. (K) meltEGFR-37 activation visualized through the live-cell ErkKTR reporter. Nuclear depletion of ErkKTR indicates Erk activation while nuclear enrichment indicates Erk inactivation. Scale bar represents 10 μm. (L) Quantification of ErkKTR activity (cyto/nuclear ratio) in HEK 293T cells expressing meltEGFR-37 or wt cells. Traces represent mean+/−1 SD of ˜15 cells per condition. (M-O) Temp-regulated proteolysis (above 37° C.). (M) Control of proteolysis at mammalian temperatures with meltTEVp-40. (N) Representative images of FlipGFP signal in cells expressing meltTEVp-40 or TEVp after incubation at the indicated temperatures for 24 hours. Scale bar represents 10 μm. (O) Quantification of FlipGFP signal in fixed cells expressing meltTEVp-40 or TEVp cultured at the indicated temperatures for 24 hours. Each bar represents the mean+/−1 SEM of ˜1000 cells. Y-axis represents mean fluorescence subtracted by the signal of TEVp-negative cells. (P-R) Temp-regulated nuclear localization (above 37° C.). (P) Control of nuclear translocation at mammalian temperatures with meltNLS/NES-40. (Q) Representative images of nuclear translocation. Scale bar represents 20 μm. (R) Quantification of nuclear localization of meltNLS/NES-40 or Melt-40-mCh after exposure to cycles of 37° C. and 41° C. (red) in HEK 293T cells. Traces represent the mean+/−1 SEM of ˜1000 cells.

FIGS. 34A-J show graphs and images illustrating thermal regulation of cell fate using Melt. (A-D) Temp-regulated cytoskeletal rearrangements. (A) Control of Cdc42 activity 617 and cell shape through recruitment of the DHPH domain of ITSNI to the membrane. (B)

Representative images of cell shape changes in response to temperature control in a HEK 293T cell transiently expressing meltITSN1-37. Upon reduction of temperature from 41° C. to 37° C., cells show rapid formation of membrane extensions and dramatic increase in size. Scale bars =20 μm. (C) Cell shape changes are reversible and repeatable over several hours of stimulation. Representative images of HEK 293T cells transiently transfected with meltITSN1-37, cultured at 41° C and exposed to multiple rounds of heating and cooling at the times and temperatures indicated. Scale bars =20μm. (D) Quantification of cell area of cells expressing either meltITSN1-37 or Melt-37 after repeated cooling and heating. Bars represent the average cell size of 15 cells+/−1 SD. (E-J) Temp-regulated cell death. (E) Thermal control of cell death through regulation of caspase-1 clustering (meltCasp1-37). (F) meltCasp1-37 induces cell death upon lowering temperature below 37° C. (G-H) Representative images of cells expressing meltCasp1-37 (G) or Melt-37 (H) before and after exposure to 34° C for 8 hours after culture at 38° C. for 24 hours. Bottom panels of (G,H) show AnnexinV-647 staining, which indicates cell death. Scale bars =40 μm. (I-J) Quantification of AnnexinV intensity in meltCasp1-37 (I) and Melt-37 (J) cells over time at the indicated temperature after prior culture at 38° C. for 24 hours. Plots represent the mean+/−SEM of per-image AnnexinV fluorescence divided by total GFP fluorescence (to account for cell density) across 4 images. See Methods for quantification details. All images/data in this figure were collected from transient expression of Melt constructs in HEK 293T cells.

FIG. 35 shows a schematic of workflow for quantification of membrane association. Experiments were performed in cells stably expressing a GFP-CAAX membrane marker. Image analysis was performed using the MorphoLibJ plugin for ImageJ to enable segmentation of cell borders. The resulting segmentation was imported into CellProfiler which was then used to filter out objects below a size threshold, eliminating cell fragments, and cells not expressing mCherry (used as a fluorescent tag for all Melt constructs quantified). From there, segmented cells could be used to quantify total mCh levels in each cell. Additionally, a 1 pixel radius at the edge of the object was assigned as the cell membrane and was used to quantify membrane levels of mCh.

FIGS. 36A-C show graphs illustrating normalization of meltTEVp proteolysis to account for temperature-dependent changes in protein expression. (A) Total protein expression is elevated at low temperatures as demonstrated by mCh-TEVp expression. Cells were cultured at the indicated temperature for 24 hours. (B) To account for changes in FlipGFP signals caused by temperature dependent expression differences, negative control (no TEVp) and positive control (constitutively membrane bound TEVp-CAAX) cells were used to establish minimal and maximal FlipGFP signals at each temperature. (C) Minimal and maximal cutting ranges at each temperature were used to normalize meltTEVp and TEVp proteolysis to the ranges established in (B) (subtracting minimum signal and dividing by maximum). This normalization was performed to account for changes in protein expression levels that could account for increases in proteolysis at low temperatures. Each bar in all plots represents the mean+/−1 SEM of ˜1000 cells. FIGS. 37A-D show graphs and images illustrating different NLS/NES combinations achieve varying levels of nuclear shuttling. (A) Diagram of all meltNLS/NES fusions tested in order to achieve the largest dynamic range of nuclear shuttling between 27° ° C. and 37° C. (B) Amino acid sequence of NLS and NES used in meltNLS/NES fusions. (C) Quantification of nuclear Melt signal using the five constructs shown in A exposed to repeated cycles of heating and cooling. Traces represent the mean of ˜1000 cells+/−SEM. (D) Representative images of meltNLS/NES combinations before and after heating to 37° C. and cooling to 27° C.

FIGS. 38A-C show graphs illustrating kinetics of membrane dissociation and reassociation 840 of Melt-PB fusions. (A) Quantification of membrane dissociation at the indicated temperature after prior culture at 27° C. for 24 hours. Dashed lines indicate the time at which the temperature was raised to the indicated temperature. (B) Quantification of membrane recruitment of the indicated construct cultured at 27° C. after previous culture at the indicated temperature for the preceding 6 hours. Traces represent the kinetics of membrane reassociation and are continuations of traces found in (A). Dashed lines indicate the time at which the temperature was lowered from the indicated temperature. (C) Quantification of membrane recruitment of the indicated construct during culture at 37° C. after culture at 27° C. for 24 hours. Dashed lines indicate the time at which cells were returned to 27° C. to identify the effect of different periods of heating on membrane reassociation kinetics. All traces represent the mean of ˜1000 cells+/−SEM. Membrane binding for all plots was normalized to the first time point of each condition.

FIG. 39 shows a graph illustrating relative membrane binding of Melt variants. Unnormalized plots 855 of data shown in FIG. 33H, showing relative membrane binding strength of Melt-30/32/37/40 at the indicated temperatures. Each point represents the average of three wells+/−SD with ˜500 cells quantified in each well.

FIGS. 40A-C show graphs illustrating kinetics of membrane dissociation and reassociation of Melt variants. (A) Quantification of membrane recruitment of the indicated construct cultured at the indicated temperatures. Traces represent the kinetics of membrane dissociation after prior culture at either 34° C. (C292A) or 37° C.(C292A+Stim) for 24 hours. Dashed lines indicate the time at which the temperature was raised to the indicated temperature. (B) Quantification of membrane recruitment of the indicated construct cultured at 34° C. (C292A) or 37° C. (C292A+Stim) after prior culture at the indicated temperature for the preceding 6 hours. Traces represent the kinetics of membrane reassociation and are continuations of traces found in (A). Dashed lines indicate the time at which the temperature was lowered from the indicated temperature. (C) Quantification of membrane recruitment of the indicated construct during culture at 41° C. after prior culture at 35° C. for 24 hours. Dashed lines indicate the time at which cells were returned to 35° C. to identify the effect of different periods of heating on membrane reassociation kinetics. All traces represent the mean of ˜1000 cells+/−SEM. Membrane binding for all plots was normalized to the first time point of each condition.

FIG. 41 shows a graph illustrating thermal activation of meltSOS-37. meltSOS-881 37 achieves signaling activation at temperatures <37° C. Plot showing quantification of pathway activation (single-cell immunofluorescence for ppErk) in cells expressing meltSOS-37 exposed to the indicated temperatures for 75 min. Data points represent the mean of 2 wells+/−SD with ˜1000 cells quantified per well.

FIG. 42 shows a graph illustrating thermal activation of meltTEVp-889 37. meltTEVp-37 achieves proteolysis at temperatures <37° C. Plot showing FlipGFP fluorescence in cells expressing meltTEVp exposed to the indicated temperatures. Data points represent the mean ˜1000 cells+/−SEM. See Example 3—Methods for FlipGFP quantification workflow.

FIG. 43 shows images illustrating quantification of cell area to assess effects of meltITSN1-897 37. A cell expressing meltITSN1-37 was imaged and subsequently thresholded in ImageJ such that cell positive pixels were set to 1 and background pixels were set to 0. A region of interest containing the cell of interest was drawn by hand. Summing the total number of positive pixels in the cell region was therefore used as a metric of total cell area.

FIGS. 44A-F show images and graphs illustrating lack of thermal stress observed below 42° C. To examine whether the temperature changes required for Melt-37/40 activation would also apply thermal stress to mammalian cells, we measured stress granule (SG) formation as well as changes in proliferation in response to thermal stimuli used throughout the manuscript. (A) SGs were visualized by immunofluorescence for G3BP1. No SGs were seen in HEK293T cells in normal growth conditions, while bright SG puncta were seen in cells treated with 100 μM sodium arsenite for 3 hours prior to fixation (positive control). (B) SGs were visualized in HEK293Ts that were exposed to various durations and intensities of heating. No SGs were observed in cells heated to <41° C., and only a few cells showed SGs when heated to 42° C. By contrast, heating to 43° C induced SGs in nearly all cells within 30 min, followed by detachment of cells at later time points. (C) To examine integration of potential heat stress over longer time periods, we measured cell proliferation. (D) Staining for phospho-Rb (pRb) indicates whether a cell is in G1 (pRb-) or in later stages of the cell cycle (pRb+). Red line in density plots separates the two populations. HEK293T cells starved for 24 hr (top plot) show a larger fraction of cells in G1 compared to cells kept in full medium (second from top). Cells cultured in full medium for 24 hr at temperatures between 38-42° C. show the same fraction of pRb+ cells as cells cultured at 37° C., indicating no stress-induced impairment of proliferation. (E) Quantification of pRb+ cells after the indicated times and temperatures of heating. Each data point represents the mean+/−SD of three wells. (F) Quantification of total cell counts from experiment in (D,E). The lack of difference in cell counts at different temperatures demonstrates a lack of changes in proliferation or cell death due to heat stimulation at or below 42° C. Each point represents the mean and range of wells. All scale bars in this figure represent 30 μm.

FIGS. 45A-H show images and graphs illustrating design and use of the thermoPlate for simultaneous, independent temperature control of multiple wells in a 96 well plate. (A) image of a fully assembled thermoPlate device for regulating temperature in individual wells of a 96 well plate. (B) Enlarged image of a single heater/reader pair from the thermoPlate. Each pair regulates the temperature of a single well of a 96 well plate. The reader is a high resistance thermistor that is able to electronically measure temperature without significantly raising the temperature of the well when powered by the operating voltage of the device (10-20V). The heater is a low resistance thermistor that generates significant heat (enough to heat 100 s of microliters of water by 10 s of degrees) when supplied with the operating voltage of the device. (C) Diagram of heater/reader operation coordinated by an arduino microcontroller. The arduino is able to take readings from a reader and use those readings to dynamically adjust the duty ratio of the heater for that well using PID feedback control. Each heater/reader pair from all 96 wells is coordinated independently by the arduino. (D) Image showing a 96 well plate coupled to the thermoPlate. (E) Raw readings taken from the reader of each well when the device is incubated in a temperature controlled incubator (incubator/ambient temperature indicated by dashed line). Each color represents readings taken from a well at a different ambient temperature. (F) Thermoplate readers can be calibrated to increase temperature accuracy and decrease deviation between readers. This process involves using the temperature readings taken in (E) to generate lines of best fit between the temperature reading from a reader and actual ambient temperature at that time. (G) The thermoPlate can be used to maintain multiple wells of a 96 well plate at different temperatures for long periods of time. 15 wells were set to 15 different temperatures in 1 degree increments (28-42° C.) with an ambient temperature of 25° C. for >16 hrs. Temperature of each well was recorded by printing the temperature measured by the reader of each well every 30 seconds. (H) The thermoPlate can also be used to generate multiple dynamic temperature patterns over time simultaneously. Here, wells were heated to 7 different temperatures for repeated cycles of 30 minutes of heating followed by 30 minutes of cooling to 28° C. with an ambient temperature of 25° C.

FIGS. 46A-F show graphs and images illustrating characterization of thermoPlate performance during heating and cooling. (A) Every well of a 96 well plate was heated by 10° C. for 20 minutes and then allowed to cool by 10° C. (from 28° C. to 38° C. and back to 28° C. with an ambient of 25° C.). Wells were heated individually/non-simultaneously. (B) The mean of all wells from (a) labeled with key metrics of thermoPlate performance. Overshoot represents the maximum temperature a well reached while heating. Accuracy is the difference between the actual temperature and desired temperature at 20 minutes. Rise time is the first time the temperature was within 0.5° C. of the desired temperature. Fall time, cooling accuracy, and undershoot represent the same parameters during the cooling phase. (C) 12 wells were heated individually by 5° C. for 20 minutes and then allowed to cool to 28° C. (with an ambient of 25° C.). This process was repeated, heating each well by 10, 15, and 20° C. The thermoPlate achieved heating by 5, 10, and 15° C., but was unable to achieve 20° C. heating in several wells, likely due to heat absorption from surrounding wells. (D) Average of each key metric from the 12 wells heated/cooled in (c) at each set temperature. In general, rise/fall time and cool time increased with increased AT while accuracy and over/undershoot peaked at ΔT=10° C. (E) The maximum temperature that can be achieved by a well is limited by the number of wells in close proximity. 3 wells spread across the plate were heated simultaneously with a desired ΔT=20° C. but were unable to achieve the desired set point in all wells. However, when 9 wells are heated in close proximity, they rapidly achieve the desired set point. (F) Characterization of the same key parameters found (d) show that these parameters vary based on the number of wells in close proximity undergoing heating. Rise time and accuracy decrease as more wells are heated while overshoot increases.

FIGS. 47A-G show graphs and images illustrating that the thermoPlate allows interrogation of stress granule dynamics in a high-throughput format. (A) The thermoPlate is compatible with microscopy and can thus be used to generate heat-induced stress granules in HeLa cells stably transduced with a G3BP-mCherry reporter while allowing simultaneous imaging. Stress granules can then be quantified to enable quantification of stress granule dynamics over time in response to dynamic heat shock conditions, revealing stress granule suppression after multiple rounds of heat shock. (B) Representative images of stress granule formation from key points in the plot found in (a). (C) The thermoPlate allows imaging of stress granule formation under multiple temperature profiles simultaneously. Specifically, we utilized the thermoPlate to examine how stress granule suppression persists and diminishes over time after an initial heat shock. We found that maximum suppression requires ˜3 hrs to achieve regardless of the number of heat pulses cells were exposed to. Suppression eventually diminished if cells were allowed to recover for 9 hrs. (D) We next asked if stress granule suppression was affected by the duration and intensity of heat shock. We used the thermoPlate to test 12 temperature conditions (3 heat shock durations by 4 temperatures). Indeed, stress granule formation was dependent on both parameters, with longer and higher temperature heat shocks both achieving higher levels of stress granule suppression 3 hours after the initial heat shock. The second heat shock was the same duration and temperature (1 hr at 43.5° C.) for all conditions. (E) Quantification of stress granule suppression (represented as percent of initial heat shock stress granule amplitude) after various lengths of recovery periods. Stress granule suppression is maximal after 3 hours and diminishes significantly after 9 hours. (F) Heatmap of the temperature/duration landscape affecting stress granule suppression. Stress granules are maximally suppressed after 1 hr of heating to any temperature, with suppression being sensitive to heating intensity at shorter heat shock durations. (G) Stress granule deformation was sensitive to heat intensity, with significant decrease in deformation speed after return to 37° C. at higher heat shock temperatures.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

### Device

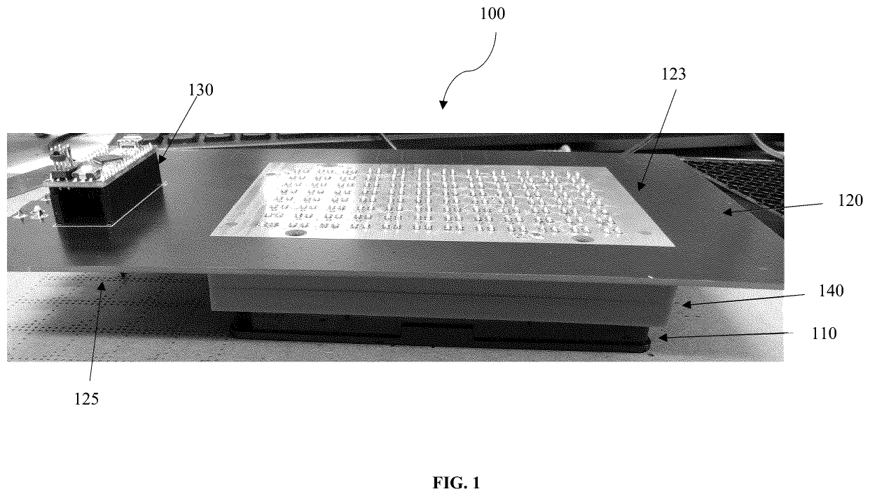

Provided herein, in some embodiments, is a device for independently controlling the temperature of individual wells in a well plate. Referring to FIG. 1, in some embodiments, the device 100 includes a well plate 110, a temperature control assembly 120, and a controller 130. The well plate 110 includes any well plate suitable for holding samples therein. In some embodiments, as illustrated in FIG. 45D, the well plate 110 includes a top well plate surface 111, a bottom well plate surface 113, and at least one well 115 formed therein. Referring to FIG. 45C, the at least one well 115 includes an opening 117 with any suitable cross-sectional shape that extends from the top well plate surface 111 to a closed bottom at or near the bottom well plate surface 113. For example, suitable well plates include known microwell plates with any suitable number of wells, such as, but not limited to, a 96-well microwell plate, a 48-well microwell plate, a 24-well microwell plate, or other well plates having any other standard or non-standard number of wells.

Turning to FIGS. 1, 2, and 45A, in some embodiments, the temperature control assembly 120, which is also referred to herein as a thermoplate, includes a circuit board 121 having a top circuit board surface 123 and a bottom circuit board surface 125, and at least one pair of thermistors 127 extending outwardly from the bottom circuit board surface 125. The circuit board 121 includes any suitable circuit board, such as, but not limited to, a printed circuit board (PCB). In some embodiments, each pair of thermistors 127 includes a heating thermistor 128 and a measurement thermistor 129. Suitable heating thermistors 128 include, but are not limited to, resistive heating thermistors.

The at least one pair of thermistors 127 is positioned on the circuit board 121 such that when the bottom circuit board surface 125 is positioned adjacent to the top well plate surface 111 (FIG. 45D), each pair of thermistors 127 extends into an individual well 115 (FIG. 45C) in the well plate 110. For example, in some embodiments, the well plate 110 includes a 96-well microwell plate and the temperature control assembly 120 includes 96 pairs of thermistors 127 (i.e., 192 thermistors), with each pair of thermistors 127 positioned to extend into a separate well 115 in the well plate 110. In such embodiments, when a liquid sample 119 is positioned in one or more of the wells 115, a portion of the corresponding pair of thermistors 127 is immersed in the sample 119 (FIG. 45C) upon positioning of the temperature control assembly 120 adjacent to the well plate 110. Accordingly, in some embodiments, the temperature control assembly 120 includes a liquid-tight covering 126 (shown generally) over the thermistors 127. In some embodiments, the liquid-tight covering 126 is individually positioned and/or formed over each thermistor 127. Alternatively, in some embodiments, the liquid-tight covering 126 is positioned and/or formed over each pair of thermistors 127. Suitable liquid-tight coverings 126 include, but are not limited to, heatshrink tubing, conformal coating, or a combination thereof. Suitable conformal coating includes, but is not limited to, silicone. For example, in some embodiments, the liquid-tight covering includes a silicone conformal coating over heatshrink tubing individually covering each of the thermistors.

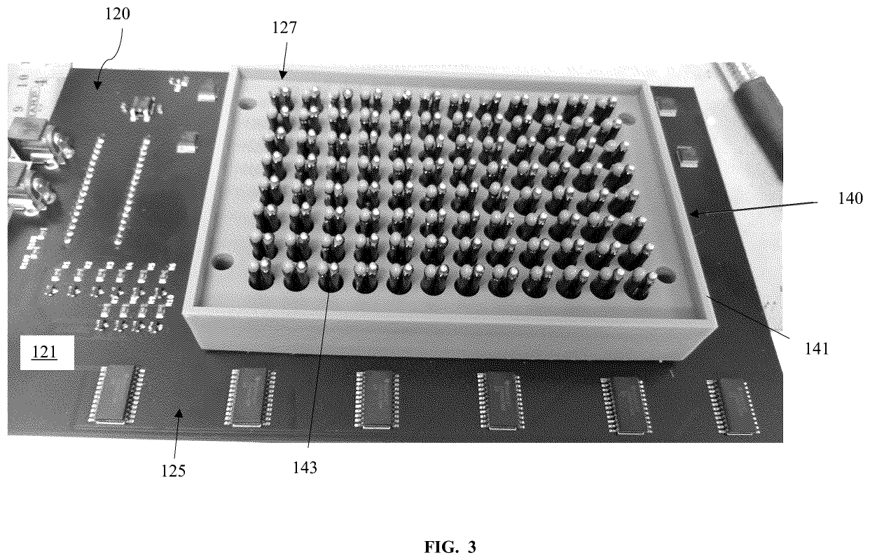

Referring to FIGS. 1, 3, 45A, and 45D, in some embodiments, the device 100 also include an adapter 140 arranged and disposed to position the temperature control assembly 120 relative to the well plate 110. The adapter 140 may be secured to or separate from the temperature control assembly 120 and includes any suitable material. Additionally, the adapter 140 includes any suitable shape and/or material for positioning the temperature control assembly 120. For example, in one embodiment, as illustrated in FIG. 3, the adapter 140 includes an outer edge 141 configured to engage the well plate 110, along with one or more inner openings 143 each arranged and disposed to permit a pair of thermistors 127 to extend therethrough. In such embodiments, a thickness of the adapter material defining the inner openings 143 may be selected to limit how far the thermistors 127 are able to extend into the wells 115. Additionally or alternatively, the inner portion of the adapter 140 may be open and/or the outer edge 141 may include one or more features (e.g., a ridge) that determine the distance between the temperature control assembly 120 and the well plate 110.

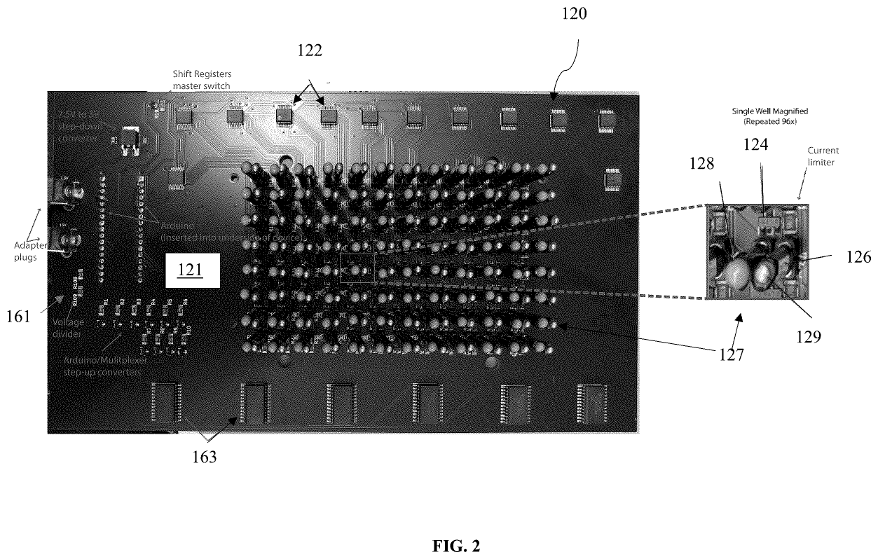

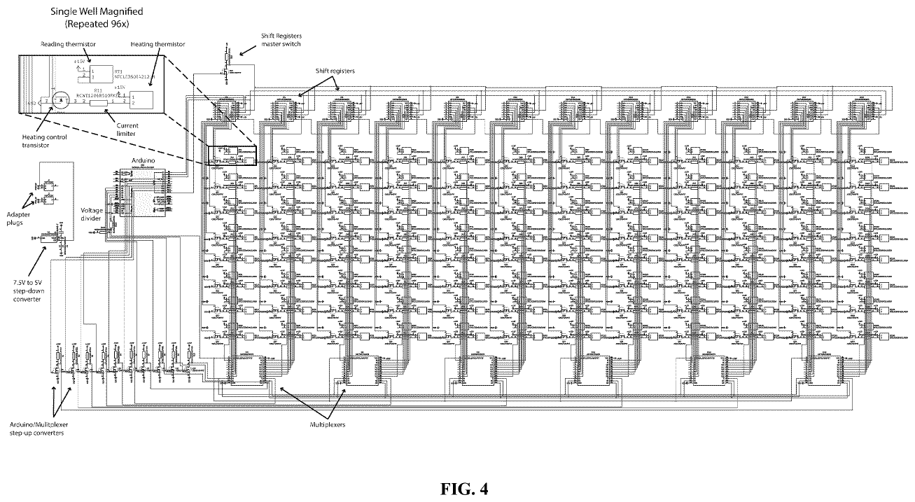

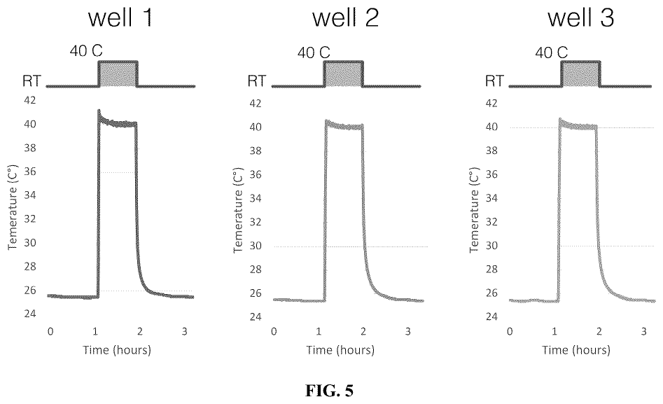

The controller 130 includes any suitable controller for individually controlling each of the thermistors 127 to independently measure and adjust the temperature within each of the wells 115. Suitable controllers 130 include, but are not limited to, a microcontroller, such as an Arduino microcontroller. Although shown in FIG. 1 as being directly coupled to the circuit board 121, as will be appreciated by those skilled in the art, the disclosure is not so limited and may include a remotely coupled controller. Referring to FIG. 2, in some embodiments, the temperature control assembly 120 includes at least one shift register 122 in communication with the controller 130, and at least one control transistor 124 in communication with the shift register 122. In such embodiments, the controller 130 communicates with the at least one shift register 122, which controls the at least one control transistor 124 to determine a current flow through one or more of the thermistors 127. Additionally or alternatively, in some embodiments, the temperature control assembly 120 includes at least one voltage divider 161 and at least one multiplexer 163 in electrical communication with the microcontroller 130. In such embodiments, the temperature of each of the at least one wells 115 is measured through the at least one voltage divider 161 and the at least one multiplexer 163. An example schematic for the temperature control assembly is shown in FIG. 4, with an individual feedback loop between the microcontroller and each of the at least one pair of thermistors.

In some embodiments, the controller includes software that allows a user to specify the temperature profile of each well, and that monitors and adjusts sample temperature using a PID control algorithm. In such embodiments, the PID algorithm continuously adjusts the duty ratio of each heater based on the controller output to maintain a user defined temperature in each well.

Also provided herein, in some embodiments, is a method of independently controlling the temperature in individual wells of a microwell plate. In some embodiments, the method includes filling one or more of the wells in a well plate with a liquid sample; positioning the device according to one or more of the embodiments disclosed herein relative to the well plate such that one or more of the at least one pair of thermistors is immersed in the liquid sample; and independently providing a current flow to each of the at least one pair of thermistors to adjust and/or measure the temperature in each of the individual wells. In some embodiments, the current flow to the heating thermistor heats the sample in the corresponding well through resistive heating. Additionally or alternatively, in some embodiments, the method includes individually heating each well by up to 15° C. in 5 minutes or less. In some embodiments, the method includes obtaining temperature readings from each well at user defined intervals. In some embodiments, the temperature readings are accurate to within 0.1° C. Furthermore, in some embodiments, the method includes running the device autonomously indefinitely.

The devices and methods disclosed herein provide both recording and automatically adjusting temperature in individual wells with high temporal resolution. Additionally or alternatively, the device and methods disclosed herein provide dynamic patterns of heating over time. Accordingly, the devices and methods disclosed herein may be used to rapidly and precisely regulate temperature for remote thermal control of proteins, cells, and tissues.

### Protein

Provided herein, in some embodiments, is a temperature-responsive protein. In some embodiments, the temperature-responsive protein includes a BcLOV4 protein variant. The BcLOV4 protein, also named BcRGS1, is from the noble rot fungus Botrytis cinerea, and includes the sequence available at GenBank accession number CCD53251.1. In some embodiments, the variant includes sequence homology with the wild-type BcLOV4 protein of at least 80%, at least 85%, at least 90%, at least 95%, between 80% and 99%, between 85% and 99%, between 90% and 99%, between 95% and 99%, or any combination, sub-combination, range, or sub-range thereof.

In some embodiments, the variant includes a point mutations at C292 and/or Q355. For example, in one embodiment, the variant includes a point mutation at Q355. In some embodiments, the point mutation at Q355 provides temperature sensitivity. In another embodiment, the variant includes point mutations at Q355 and C292. In some embodiments, the point mutation at C292 modulates the temperature response range. In some embodiments, the Q355 point mutation is Q355N. In some embodiments, the C292 point mutation includes C292A, C292R, C292N, C292D, C292E, C292Q, C292G, C292H, C292I, C292L, C292K, C292M,

C292F, C292P, C292S, C292T, C292W, C292Y, or C292V. For example, in some embodiments, the variant includes point mutations of Q355N and C292A, with the Q355N point mutation providing the temperature sensitivity and the C292A point mutation modulating the temperature response range towards mammalian temperature. Additionally or alternatively, in some embodiments, the variant includes one or more amino acid deletions as compared to the wild-type protein. For example, in some embodiments, the variant includes deletion of one or more of amino acids 1-97.

Without wishing to be bound by theory, it is believed that the Q355N point mutation places the protein in a lit-state (i.e., mimicks the effect of light activation). Additionally, and again without wishing to be bound by theory, it is believed that the C292 point mutation adjusts the activation temperature of the protein. Accordingly, in some embodiments, the membrane-localization of the variant including the Q355N and C292 point mutations is purely temperature sensitive (i.e., controlled by temperature). For example, in one embodiment, the variant including the Q355N point mutations localizes to the membrane constitutively below 27° C. and completely dissociates from the membrane as the temperature is increased from 27° C. to 34° C. In another embodiment, the variant including the Q355N and either the C292A or C292P point mutations localizes to the membrane constitutively at 37° C. and dissociates from the membrane as the temperature is increased to 42° C. As such, in some embodiments, the variant provides a range for thermoswitching in human cells and tissues that permits actuation without damaging tissues.

Also provided herein, in some embodiments, is a method of modifying cell behavior through the temperature-dependent membrane localization of the BcLOV4 variant. In some embodiments, for example, the method includes control of proteolysis at the membrane. In such embodiments, a protease is localized to the membrane at cold temperatures (depending upon the C292 mutation), where it can cleave substrates, and upon heating the variant/protease dissociates and cleavage is stopped/reduced. In some embodiments, the method includes turning signaling pathways on and off through temperature adjustment. For example, membrane localization may be used to control downstream transcription through sequestration of a transcription factor (TF) at the membrane. At low temperatures, the variant is at the membrane and transcription is off, while at elevated temperatures, the variant dissociates and translocates to the membrane to initiate transcription. In one embodiment, this temperature adjustment is used to control both Ras and receptor tyrosine kinase signaling. In some embodiments, the variant controls subcellular localization of proteins in response to temperatures. For example, the variant may shuttle proteins into and out of the nucleus with temperature.

The proteins and methods disclosed herein may be used to control any suitable protein or cell remotely and/or on demand. For example, in one embodiment, CAR-T cell therapies may be controlled remotely to specify where and when a T cell should be armed or infiltrate a tumor. In another embodiment, the timing of gene or protein activity may be controlled during a fermentation or other biomanufacturing process to optimize yields. In contrast to light-activatible control of cells, where blue light does not penetrate tissues efficiently (˜1 mm), a cell's temperature can be controlled at greater depth (˜1-10 cm) while retaining mm-scale resolution (e.g., using focused ultrasound).

Without wishing to be bound by theory, it is believed that the proteins and methods disclosed herein represent a fundamentally novel way to control proteins in the cell, in a modular, single-component format. Additionally, in some embodiments, the variant couples temperature changes to membrane localization and also clustering of a protein, two temperature-dependent protein behaviors that have not been demonstrated for a single protein. Furthermore, the protein disclosed herein is believed to be the only single-component method to control proteins and cells using temperature. This allows for more straightforward application of temperature control to engineered cells, with a smaller genetic payload and no need for stoichiometric tuning of protein components.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

We describe single-component optogenetic probes whose activation dynamics depend on both light and temperature. We used the BcLOV4 photoreceptor to stimulate Ras and phosphatidyl inositol-3-kinase signaling in mammalian cells, allowing activation over a large dynamic range with low basal levels. Surprisingly, we found that BcLOV4 membrane translocation dynamics could be tuned by both light and temperature such that membrane localization spontaneously decayed at elevated temperatures despite constant illumination. Quantitative modeling predicted BcLOV4 activation dynamics across a range of light and temperature inputs and thus provides an experimental roadmap for BcLOV4-based probes. BcLOV4 drove strong and stable signal activation in both zebrafish and fly cells, and thermal inactivation provided a means to multiplex distinct blue-light sensitive tools in individual mammalian cells. BcLOV4 is thus a versatile photosensor with unique light and temperature sensitivity that enables straightforward generation of broadly applicable optogenetic tools.

Optogenetic probes permit light-induced control of intracellular biochemistry. Such probes are typically engineered from proteins that evolved to respond to their host's environmental conditions, that is to its light status, but in some cases also to temperature. Light-responsive actuators now exist for control of protein dimerization, allostery, oligomerization, ion transport and membrane recruitment, providing an extensive toolset for precise manipulation of an array of biological processes, including cell signaling.

Ras and phosphatidyl inositol-3-kinase (PI3K) are signaling regulators that together control essential cell processes including transcription, translation, growth, survival, proliferation and migration. Optogenetic control of these two pathways has enabled recent discoveries of how their spatiotemporal dynamics regulate cell and tissue growth, form and disease. Currently, optogenetic activation of Ras or PI3K is achieved through membrane recruitment of signaling effectors via light-induced protein heterodimerization. However, this approach is limited by the necessity for two distinct proteins, which can require stoichiometric tuning of both components to permit signaling through a large dynamic range with minimal elevated basal signaling. Although stoichiometric tuning is feasible in single cells, it is more challenging in tissues and organisms.

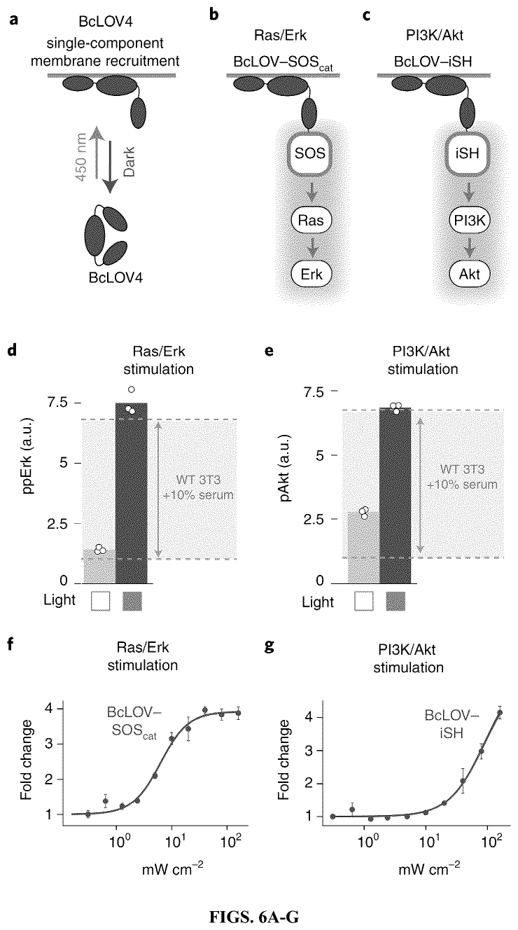

Single-component membrane translocation was recently described using the BcLOV4 photoreceptor, which translocates from the cytoplasm to membrane phospholipids under blue light in mammalian cells (FIG. 6A). BcLOV4 has already served as a modular technology for light-induced activation of the Rho GTPases Rac1, RhoA29 and Cdc42, suggesting that BcLOV4 may be adapted to regulate many additional pathways.

In this work, our initial goal was to generate and characterize BcLOV4-based probes for Ras or PI3K activation. Surprisingly, we discovered that BcLOV4 translocation and signal activation respond not only to blue light, but also to temperature, such that under sustained, long-term stimulation, BcLOV4 becomes inactivated and dissociates from the membrane as a function of increased temperature and light intensity. Through systematic characterization, we developed and validated a quantitative model that predicted BcLOV4 and downstream signaling dynamics as a function of light and temperature, providing a roadmap for BcLOV4 usage over a range of experimental conditions, particularly during long time-course experiments. We demonstrate the broad applicability and stable activation of our probes in zebrafish embryos and Drosophila Schneider 2 (S2) cells, which operate at low temperatures (22-30° C.). Finally, we demonstrate that temperature inactivation of BcLOV4 can be leveraged to allow multiplexing of blue-sensitive optogenetic probes in individual mammalian cells.

### Results

Engineering Control of Ras and PI3K Signaling with BcLOV4.

To generate an actuator of Ras/Erk signaling, we fused BcLOV4 to the catalytic domain of the Ras guanine nucleotide exchange factor Son of Sevenless 2 (SOS_{cat}), which activates Ras upon recruitment to the membrane (FIG. 6B). We generated an analogous probe to control PI3K signaling by replacing the SOS_{cat} domain with the inter-SH2 domain of the p85 subunit (iSH) (FIG. 6C). We refer to these probes as BcLOV-SOS_{cat} and BcLOV-iSH, respectively.

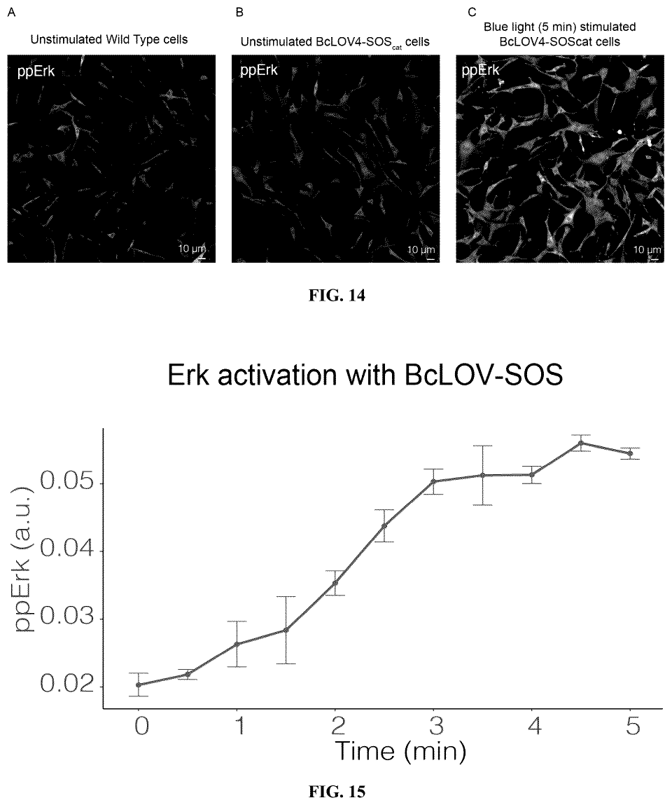

To test probe activity, we illuminated NIH 3T3 cells that stably expressed either BcLOV-SOS_{cat} or BcLOV-iSH, and we quantified levels of phospho-Erk (ppErk) or phosho-Akt (pAkt) using immunofluorescence imaging (FIGS. 14A-C). In the absence of blue light, BcLOV-SOS_{cat} cells exhibited low basal levels of ppErk, similar to wild-type (WT) cells. Upon illumination, ppErk levels rose dramatically and reached their peak within 5 min (FIG. 15), reaching levels comparable with WT cells stimulated with 10% serum (FIG. 6D). BcLOV-iSH-expressing cells also induced strong levels of pAkt, although basal activation in the absence of light was somewhat higher relative to WT cells (FIG. 6E).

These results suggest that our BcLOV-derived probes stimulate physiologically relevant levels of pathway activation while minimally disrupting endogenous cell physiology in the dark state.

To better characterize light-induced stimulation of BcLOV OS_{cat} and BcLOV-iSH, we measured the dose-response of signal activation as a function of light intensity. We found that BcLOV-SOS_{cat} achieves half-maximal pathway stimulation with 7 mW cm⁻² of blue light and saturates near 40 mW cm⁻² with a fourfold signal induction (FIG. 6F). Conversely, we estimate that BcLOV-iSH reaches half-maximal signal induction with 80 mW cm⁻², although we did not reach saturation at the highest levels of stimulation (160 mW cm⁻²) (FIG. 6G). Taken together, given the large fold-change induction, minimal basal level of stimulation and single-component nature, BcLOV4-based probes offer several beneficial qualities for optogenetic stimulation of Ras and PI3K signaling.

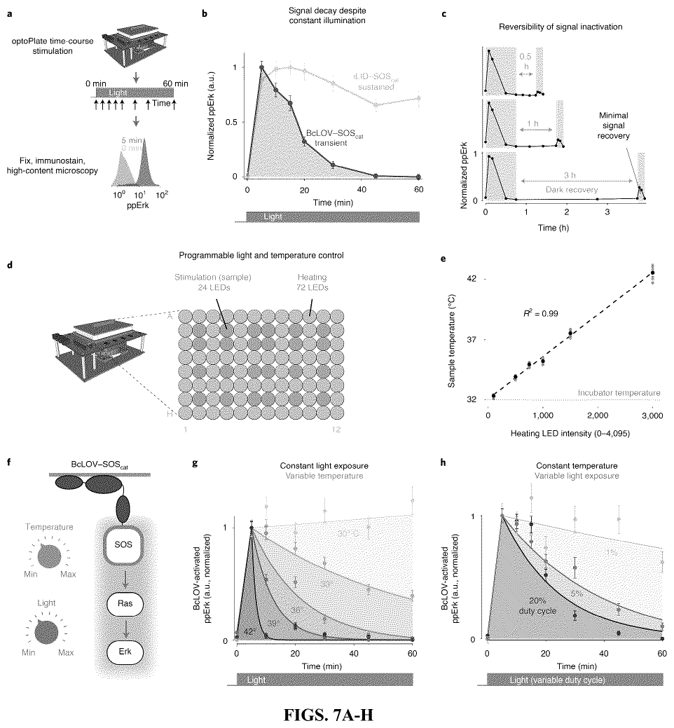

Optogenetic activation decays during extended stimulation. We next asked how BcLOV4-based probes regulate signaling through time. We used recently described illumination devices for microwell plates (the optoPlate-96) to perform time-course stimulation experiments. After stimulation, cells were immunostained and quantified to assess pathway activity (FIG. 7A).

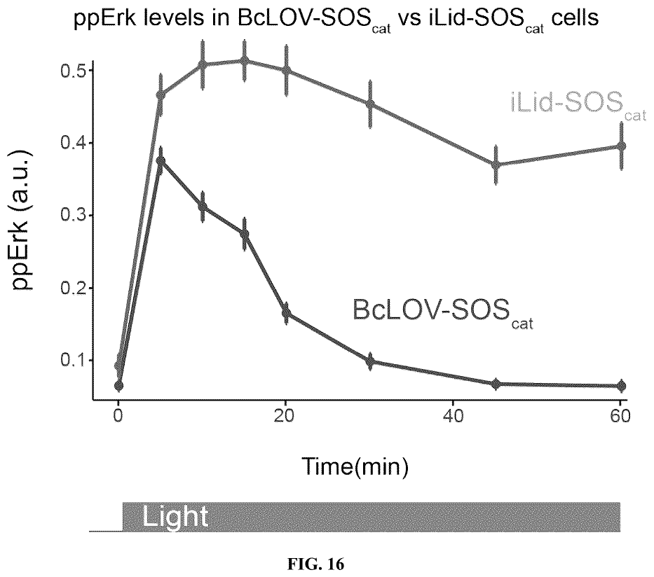

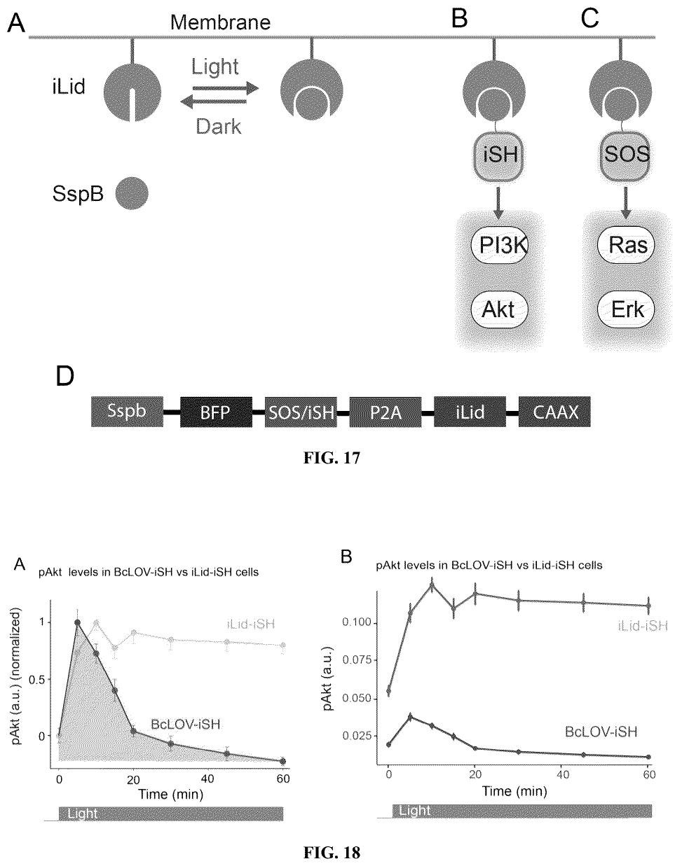

We were surprised to find that, despite constant stimulation for 60 min, cells that expressed BcLOV-SOS_{cat} showed an initial increase followed by rapid and complete decay of ppErk (FIGS. 7B and 16). To determine whether transient signaling was caused by BcLOV4 or alternative mechanisms (for example, negative feedback within the Ras/Erk pathway), we compared activation dynamics with those achieved with an orthogonal optogenetic system, iLID/sspB (nano), a commonly used blue-light inducible heterodimerizing protein pair6, which can be adapted to recruit SOS_{cat} to the membrane (iLID-SOS_{cat}23; FIG. 17). By contrast to BcLOV-SOS_{cat}, ILID-SOS_{cat} produced stable signaling under identical illumination conditions (see Supplementary Table 1 for detailed experimental conditions for all experiments), indicating that Ras/Erk signal decay was a feature of BcLOV4 stimulation. Similarly, sustained illumination of BcLOV-iSH cells led to an initial increase followed by a rapid decrease in pathway activity, whereas stimulation with an analogous iLID-based probe (iLID-iSH; FIG. 17) resulted in sustained activity (FIGS. 18A-B). These results further indicate that transient activation dynamics were a function of BcLOV4 activation and not the pathway under study.

SUPPLEMENTARY TABLE 1
Illumination and culture conditions for all experiments

| | Temp (° C.) | Duty Cycle | Intensity | | ON time | | OFF time | | humidified | CO2 |
|---|---|---|---|---|---|---|---|---|---|---|
| 1D | 37 | 20.0% | 160 | mW/cm2 | .5 | s | 2.5 | s | Yes | 5% |
| 1E | 37 | 20.0% | 160 | mW/cm2 | .5 | s | 2.5 | s | Yes | 5% |
| 1F | 37 | 100% | variable | | const. | | — | | Yes | 5% |
| 1G | 37 | 100% | variable | | const. | | — | | Yes | 5% |
| 2B | 37 | 20.0% | 160 | mW/cm2 | .5 | s | 2.5 | s | Yes | 5% |
| 2C | 37 | 20.0% | 160 | mW/cm2 | .5 | s | 2.5 | s | Yes | 5% |
| 2G | variable | 20.0% | 160 | mW/cm2 | .5 | s | 2.5 | s | Yes | 5% |
| 2H | 37 | variable | 160 | mW/cm2 | .5 | s | variable | | Yes | 5% |
| 3C | variable | 3.3% | 1.45 | W/cm2 | 1 | s | 30 | s | Yes | 5% |
| 3D | 37 | variable | 1.45 | W/cm2 | variable | | 30 | s | Yes | 5% |
| 4C | 37 | 20.0% | 160 | mW/cm2 | .5 | s | 2.5 | s | Yes | 5% |
| 4D | variable | variable | 160 | mW/cm2 | .5 | s | variable | | Yes | 5% |
| 5A/B | 25 | 3.3% | 1.45 | W/cm2 | 1 | s | 30 | s | Yes | 5% |
| 5D/E | 25 | 3.3% | 1.45 | W/cm2 | 1 | s | 30 | s | Yes | 5% |
| 5F/G | 25 | 3.3% | 1.45 | W/cm2 | 1 | s | 30 | s | Yes | 5% |
| 5H | 25 | 20.0% | 160 | mW/cm2 | .5 | s | 2.5 | s | Yes | 5% |
| 6B/C | 37 | 3.3% | 1.45 | W/cm2 | 1 | s | 30 | s | Yes | 5% |
| 6E Short/Long Light | 30 | 0.3% | 1.45 | W/cm2 | 0.1 | s | 30 | s | Yes | 5% |
| 6E Inactive + Long Light | 37 | 3.3% | 1.45 | W/cm2 | 1 | s | 30 | s | Yes | 5% |
| S1 | 37 | 20.0% | 160 | mW/cm4 | .5 | s | 2.5 | s | Yes | 5% |
| S2 | 37 | 20.0% | 160 | mW/cm2 | .5 | s | 2.5 | s | Yes | 5% |
| S3 | 37 | 20.0% | 160 | mW/cm2 | .5 | s | 2.5 | s | Yes | 5% |
| S5 | 37 | 20.0% | 160 | mW/cm2 | .5 | s | 2.5 | s | Yes | 5% |
| S6 | 37 | 20.0% | 160 | mW/cm2 | .5 | s | 2.5 | s | Yes | 5% |
| S8A-D | variable | 20.0% | 160 | mW/cm2 | .5 | s | 2.5 | s | Yes | 5% |
| S8E | 37 | 20.0% | 160 | mW/cm2 | .5 | s | variable | | Yes | 5% |
| S9 | variable | 3.3% | 1.45 | W/cm2 | 1 | s | 30 | s | Yes | 5% |
| S10 | variable | 3.3% | 1.45 | W/cm2 | 1 | s | 30 | s | Yes | 5% |
| S11 | variable | 3.3% | 1.45 | W/cm2 | 1 | s | 30 | s | Yes | 5% |
| S12 | 37 | 3.3% | 1.45 | W/cm2 | 1 | s | 30 | s | Yes | 5% |
| S14 | variable | variable | 160 | mW/cm2 | .5 | s | variable | | Yes | 5% |
| S15 | 37 | 50% | 160 | mW/cm2 | .5 | s | 1 | s | Yes | 5% |

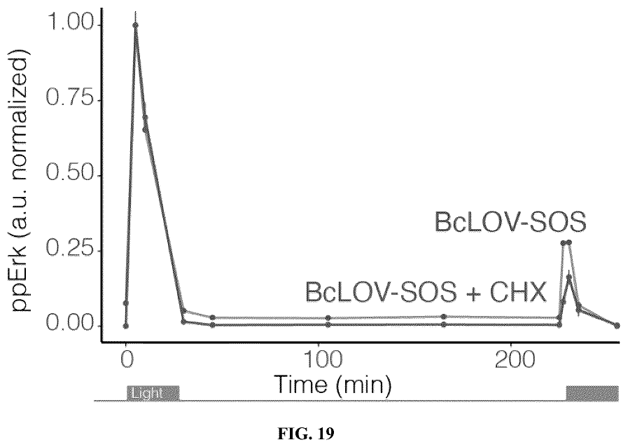

To understand the nature of signal decay, we performed a series of experiments using the BcLOV-SOS_{cat} probe. We first asked whether BcLOV-SOS_{cat} inactivation could be reversed after removal of the light stimulus. We stimulated cells with blue light until the signal decayed, withdrew light for either 0.5, 1 or 3 h and then restimulated for 10 min (FIG. 7C). As before, the initial 45 min of blue light led to a pulse of ppErk signal. However, only ˜10% of the original signal could be obtained upon restimulation even after 3 h of light withdrawal. This small amount of reactivation was partially a result of new protein production rather than reversal of inactivated BcLOV-SOS_{cat} (FIG. 19). These results suggest that BcLOV4 can undergo a spontaneous transition into an uncharacterized, long-lived inactivated state.

We next asked whether certain experimental parameters could modulate the observed signal decay rate. We noticed that pathway decay kinetics could change as a function of the illumination settings of individual experiments. Because higher light intensity can also cause heating of the sample, we tested the effects of both temperature and light on BcLOV-SOS_{cat} stimulation kinetics. To perform optogenetic time-course experiments at specific temperatures, we adapted the optoPlate-96 to precisely control both illumination and sample temperature (FIGS. 7D-E and 20A-G). Briefly, we decoupled sample heating from the illumination profile by designating 24 light-emitting diode (LED) positions for light stimulation and using the remaining 72 LED positions as heating elements (FIG. 7D). Illumination of the 72 ‘heater’ LEDs over 3,000 intensity levels correlated linearly with sample temperature in the wells above the 24 ‘stimulation’ LEDs (R2=0.99) (FIG. 7E).

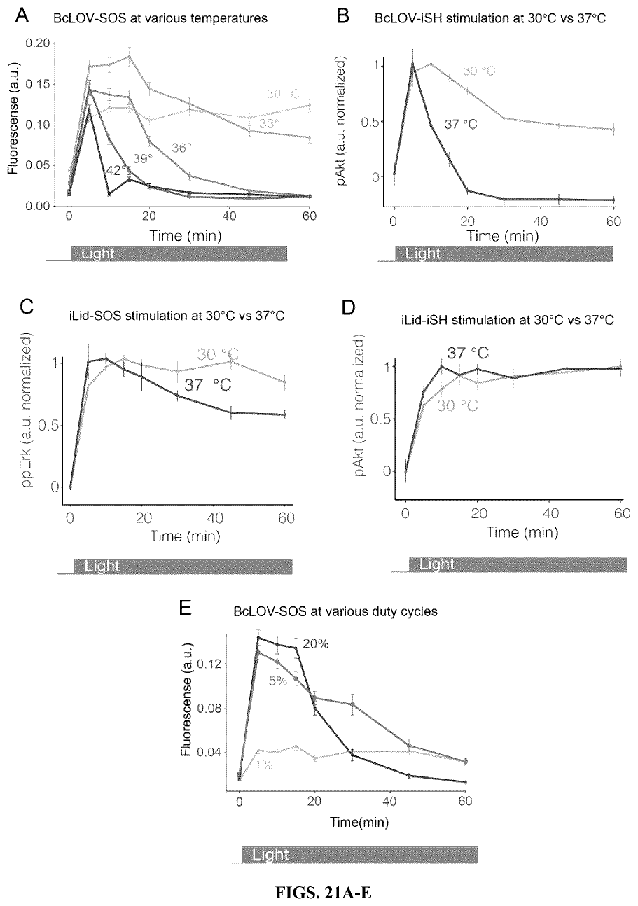

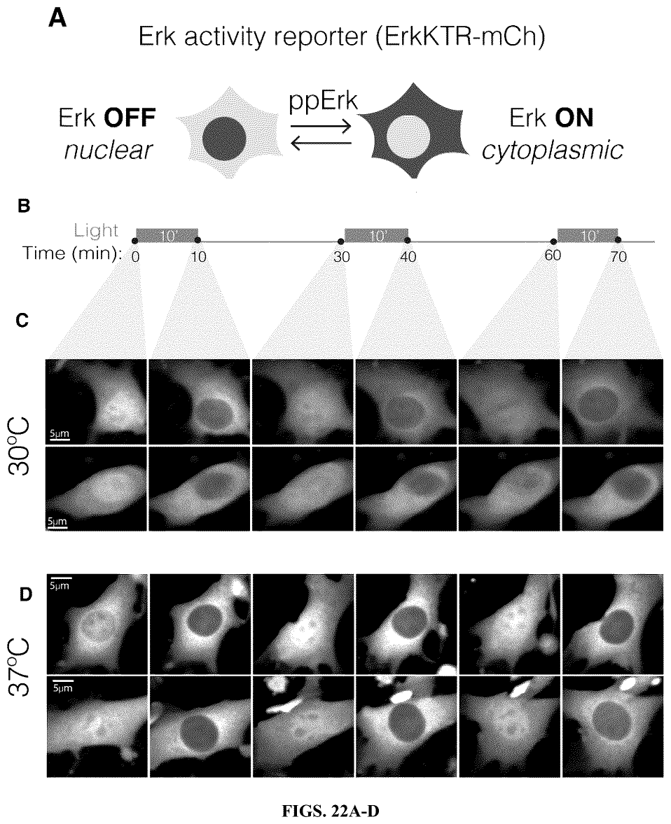

Strikingly, we observed that BcLOV-SOS_{cat} decay kinetics were strongly correlated to sample temperature, where signal decay was faster at higher temperatures (FIGS. 7F-G and 21A). At the extremes, ppErk decayed with a half-life of ˜6 min at 42° C., whereas decay was minimal at 30° C. We observed strong ppErk signal induction at all temperatures tested, suggesting that temperature only impacted BcLOV4 function in its lit, activated form (FIG. 21A). The BcLOV-iSH probe was also temperature-sensitive (FIG. 21B). By contrast, iLID-SOS- and iLID-iSH-induced signaling was sustained at both high and low temperatures (FIGS. 21C-D), further indicating that temperature sensitivity is a property of BcLOV4 control. Finally, we used a live-cell reporter of Erk activity (ErkKTR32) as an orthogonal readout to verify that, in contrast to temperature-regulated inactivation, optogenetic inactivation (removal of blue light) was reversible over multiple illumination cycles at both high (37° C.) and low (30° C.) temperatures (FIGS. 22A-D).

We also observed that the BcLOV-SOS_{cat} signal decay rate was dependent on light intensity, where higher intensity led to rapid decay, whereas lower intensity led to more sustained stimulation (FIGS. 7H and 21E). The low light achieved sustained signaling at the expense of signal amplitude (FIG. 21E), although we note that this tradeoff will be specific to the pathway under study. For example, in a previous report, comparable sparse illumination conditions (1.6% duty cycle) yielded saturating activation levels of BcLOV-regulated Rho GTPase signaling.

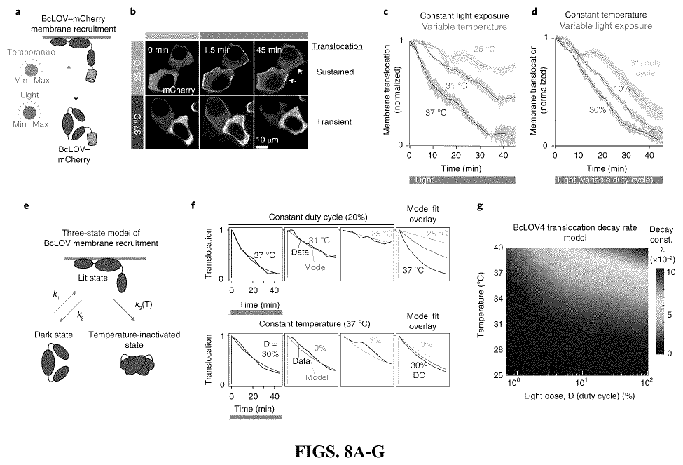

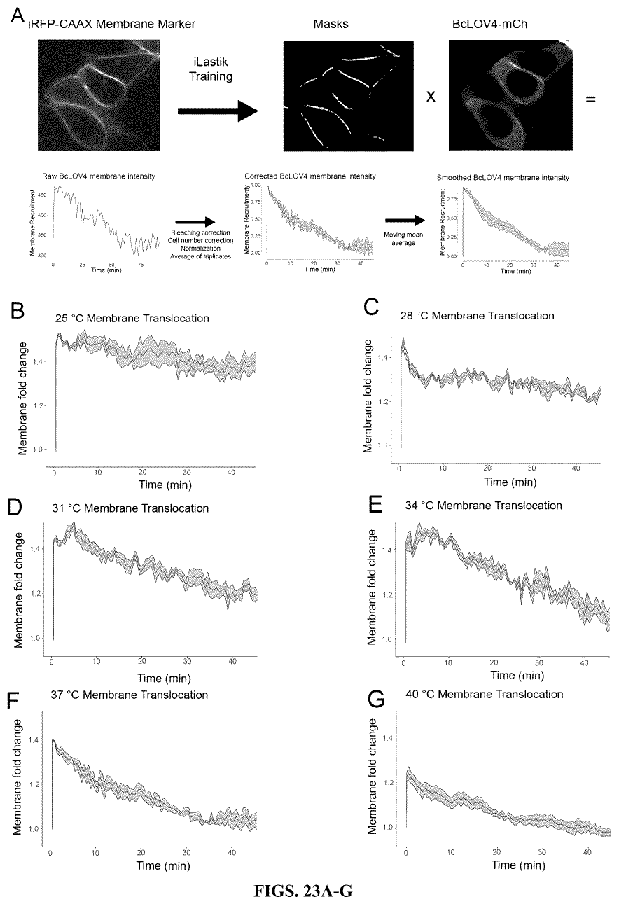

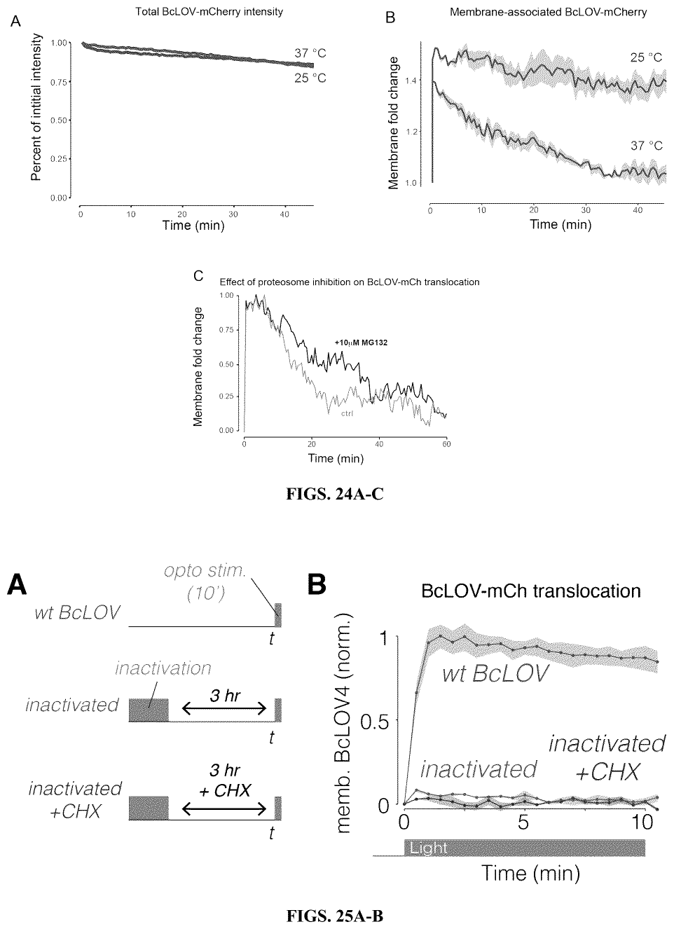

Because signal decay was observed with both the BcLOV-SOS_{cat} and BcLOV-iSH probes, but not with analogous iLID-based probes (FIGS. 7B and 21C), we suspected that the observed light- and temperature-dependent decay kinetics were a property of the BcLOV4 photosensor itself. We thus quantified membrane translocation of BcLOV-mCherry under various light and temperature conditions (FIGS. 8A and 23A-G). In accordance with our signaling results, sustained illumination resulted in sustained membrane localization at low temperatures (25° C.) but only transient localization at 37° C., with inactivated BcLOV4 returning to the cytoplasm after ˜30 min (FIGS. 8B-C and 23A-G). Similarly, when we varied light intensity at a constant temperature, increased intensity (duty cycle) increased the decay rate of membrane translocation (FIG. 8D). Decay of membrane fluorescence was not due to BcLOV4 degradation (FIGS. 24A-C). As before, we observed initial membrane recruitment at all temperatures (FIGS. 23A-G), and BcLOV4 inactivation was irreversible for at least 3 h (FIGS. 25A-B). Together, these data demonstrate that BcLOV4 is not only a photosensor, but also a temperature sensor.

Modeling dependence of BcLOV on temperature and light. To explain and predict BcLOV-mCherry translocation dynamics, we developed a computational model. We reasoned that, in addition to the dark and lit states, a third state of BcLOV4 could account for our observations of light- and temperature-dependent decay kinetics (FIG. 8E). This third state, which we call the temperature-inactivated (TI) state, is only accessible from the lit (membrane-bound) state, and transition to the TI state is irreversible at the timescales we consider (FIGS. 7C, 19, and 25A-B). We predicted that the rate of entry into the TI state (k₃) would increase as a function of temperature. Thus, because more light increases the amount of BcLOV4 in the lit state, and higher temperature increases the transition rate from the lit to the TI state, our model could explain why both increased light and temperature can increase the decay rate of BcLOV4 membrane translocation. For details on model development, see FIGS. 8E-G and 23A-G, and Methods.

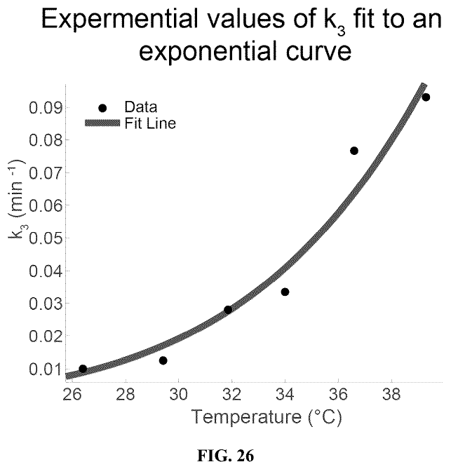

We parameterized our model by fitting values for k₁, k₂ and k₃ to live-cell data of BcLOV-mCherry translocation dynamics over a range of temperatures (25-40° C.) and light exposures (1.1%, 3.3%, 10% duty cycle) (FIGS. 8E-F, 23A-G, and 26). Across experimental conditions, we obtained consistent values for k₁ and k₂ (Methods), which correspond closely to reported values of BcLOV4 membrane translocation and dissociation (half-times of ˜1 s for association and ˜1 min for dissociation). As expected, k₃ showed a strong exponential dependence on temperature (FIG. 26). We then used our parameterized model to generate a two-dimensional heatmap of predicted BcLOV4 decay rate as a function of temperature and light dose during sustained stimulation (FIG. 8G). This heatmap represents a roadmap of translocation dynamics that can be used to predict the behavior of BcLOV4-based probes.

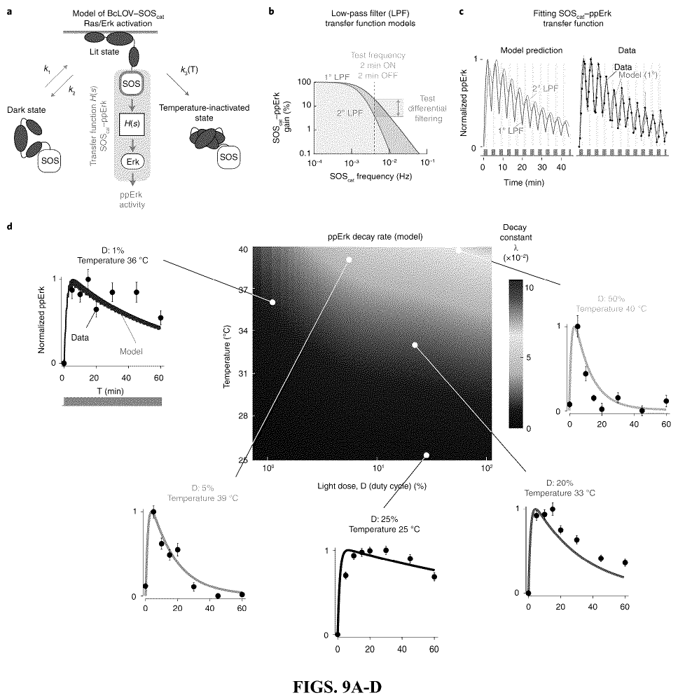

To validate our model and predict signaling dynamics downstream of BcLOV-SOS_{cat} stimulation, we integrated our model of membrane translocation with a model of Ras/Erk signal transmission (FIG. 9A). We modeled the Ras/Erk pathway with a transfer function that represents signal transmission from membrane-localized SOS_{cat} to Erk phosphorylation as a dynamic filter. Previous work defined the Ras/Erk module as a second-order low-pass filter (LPF) with a 2-mHz cutoff frequency. However, this previous work applied SOS_{cat} membrane localization as an input and measured nuclear localization of fluorescently tagged Erk2 as the output, whereas in our case the output was cytoplasmic levels of endogenous ppErk. Thus, to choose the most appropriate model, we performed dynamic stimulation of BcLOV-SOS_{cat} and fitted either first- or second-order LPF models to the data. We found that signal transmission was best modeled by a first-order LPF with a cutoff frequency of 2 mHz (FIGS. 9B-C). Because first-order filters transmit fast signal dynamics more efficiently than analogous second-order filters (FIG. 9B), our results suggest that the Ras/Erk pathway can transmit fast signal fluctuations (less than ˜4 min) more effectively than previously measured. Our results may differ from previous measurements because of additional biochemical steps required to transduce phosphorylation into nuclear translocation of fluorescently tagged Erk, compared with the direct observation of Erk phosphorylation in our work. Notably, our model captured both the fast timescale dynamics of ppErk fluctuations, as well as the slow timescale decay of ppErk due to progressive BcLOV4 inactivation (FIG. 9C).

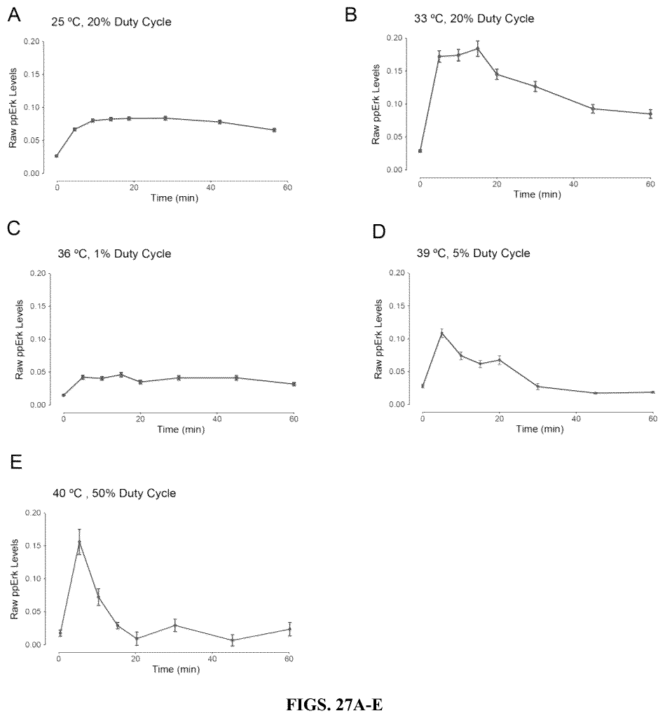

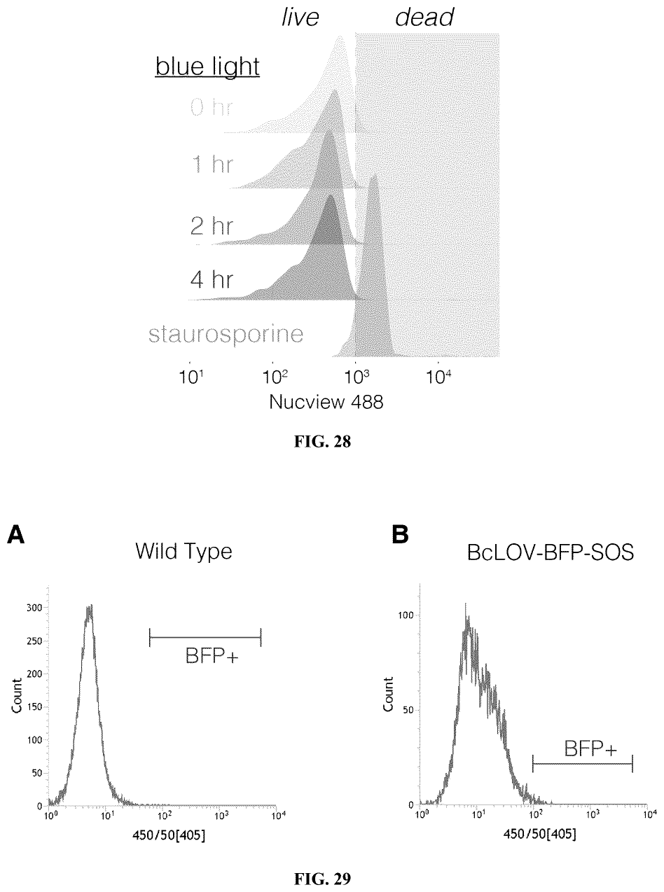

Our integrated model of membrane translocation and Erk activation predicted how specific light and temperature inputs shape BcLOV-SO_{cat}-induced ppErk dynamics. We used this model to generate a heatmap of ppErk decay rate as a function of temperature and light dose during constant illumination (FIG. 9D). To validate our model, we performed stimulation time-course experiments at temperature and light conditions that were sampled from regions of our heatmap with diverse decay rates. Measured ppErk decay rates matched closely with the rates predicted by our model over all experimental conditions tested (FIGS. 9D and 27A-E). Phototoxicity was not observed under the illumination conditions used in this study (FIG. 28).

Together, our data and models comprehensively describe how BcLOV4 and optogenetic probes thereof will behave as a function of light and temperature condition. We note, however, that decay rates will probably vary between pathways due to pathway-specific biochemistry and must thus be determined empirically.

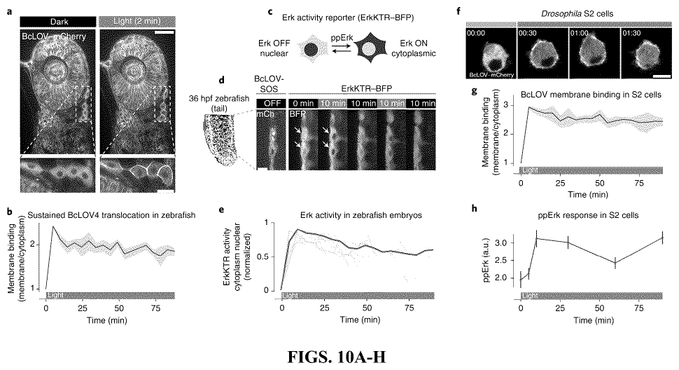

BcLOV4-based signal activation in model organisms. The single-component nature and low rate of spontaneous decay at <30° C. position BcLOV4-based tools as highly suited for experiments in tissues and model organisms that operate at lower temperatures. We thus tested the performance of BcLOV4 and BcLOV-SOS_{cat} in both zebrafish embryos and Drosophila S2 cells. BcLOV-mCherry expressed well in zebrafish embryos and, upon illumination, rapidly translocated to the membrane in all cells (FIG. 10A). Membrane translocation was sustained through 90 min of illumination, as expected from our experiments in mammalian cells (FIG. 10B). To determine whether BcLOV-SOS_{cat} could stimulate Ras signaling in zebrafish, we coexpressed BcLOV-SOS_{cat} with the ErkKTR reporter, which has previously been used in zebrafish (FIG. 10C). In cells that coexpressed BcLOV-SOS_{cat} and ErkKTR, we observed rapid and reversible ErkKTR translocation that could be stimulated over multiple cycles (FIG. 10D), consistent with our data from mammalian cells. Notably, over 90 min of sustained stimulation, Erk activity remained high, demonstrating that both BcLOV-SOS_{cat} translocation and signal activation could be maintained (FIGS. 10A-H). We also expressed BcLOV4 probes in Drosophila S2 cells as an orthogonal model system that grows at temperatures permissive to stable BcLOV4 translocation, and we again observed sustained BcLOV- mCherry membrane localization through 90 min of blue-light illumination (FIGS. 10F-G). Furthermore, expression and sustained stimulation of BcLOV-SOS_{cat} allowed sustained activation of ppErk (FIG. 10H). Together, these data show that BcLOV4-based probes can serve as simple and sensitive optogenetic probes across diverse cells, tissues and organisms of study.

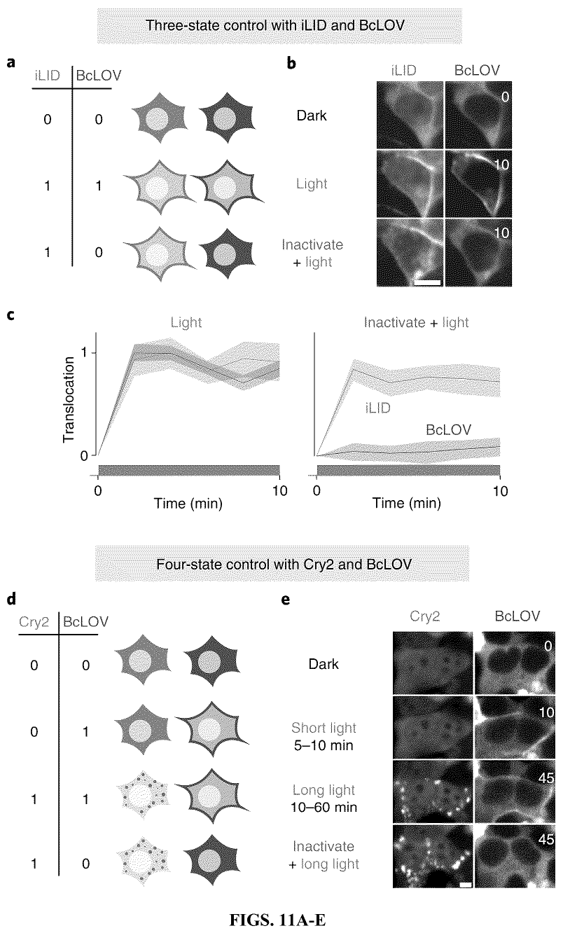

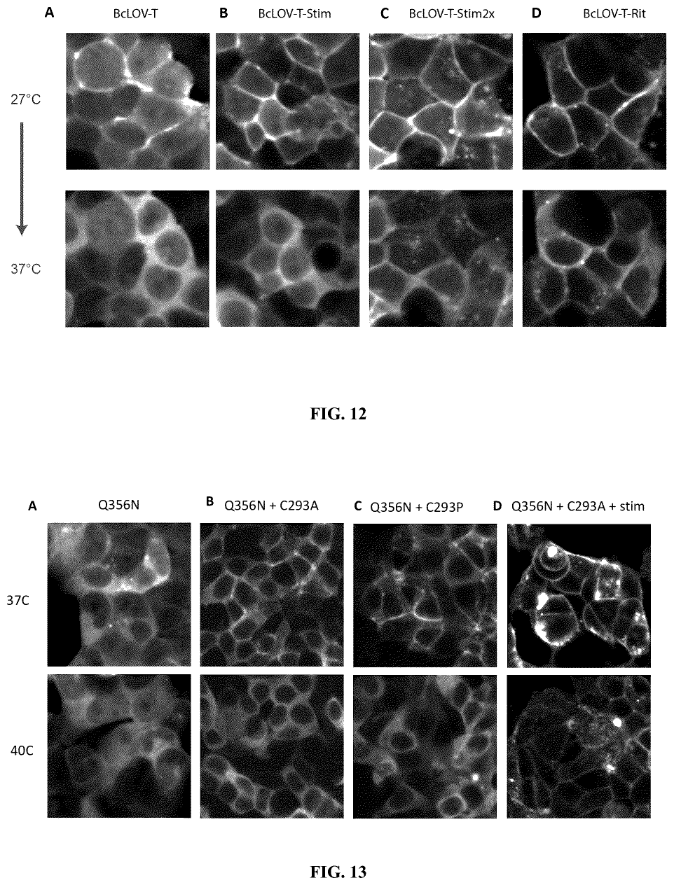

Optogenetic multiplexing using BcLOV4. Finally, we reasoned that the unique light-and temperature-responsiveness of BcLOV4 could be leveraged as a control mode to regulate multiple optogenetic proteins in single cells. Currently, such multiplexing can be achieved using optogenetic probes with different activation spectra (for example, blue- and red-absorbing). However, there is a relative lack of optogenetic proteins that respond to red-shifted (nonblue) light, and the absorption spectra of these few probes can also reach into the 400-500 nm (blue) range, thus challenging orthogonal multiplexing with blue-sensitive probes. An arguably simpler approach would be to multiplex control of distinct blue-sensitive probes. To demonstrate how temperature regulation of BcLOV4 enables such multiplexing, we coexpressed BcLOV4 with one of two blue-light sensitive tools: an iLID/sspB membrane binding system (FIG. 17) or cryptochrome 2 (Cry2), which forms large aggregates when activated by blue light. Coexpression of BcLOV4-mCherry and green fluorescent protein (GFP)-tagged iLID/sspB (iLID/sspB- GFP) allowed control of three activation states: none (dark), both (blue light) or iLID-ONLY, achieved by sequential heat inactivation of BcLOV4 and subsequent light stimulation of iLID (FIGS. 11A-C).

Coexpression of BcLOV-GFP with Cry2-mCherry allowed control of all four possible activation states (FIGS. 11D-E). In this arrangement, the BcLOV-ONLY state can be achieved because of differential ON-kinetics of fast BcLOV4 translocation versus the slower formation of large Cry2 clusters. We note that although the Cry2-mCherry displayed in FIG. 11E was imaged at 45 min, the time at which clusters appear is highly variable (approximately minutes to tens of minutes) and depends on Cry2 concentration. Nevertheless, because Cry2 clustering kinetics are slower than BcLOV translocation (approximately seconds), they permit a BcLOV-ONLY state at relatively short timescales.

### DISCUSSION

We describe the application of BcLOV4 membrane translocation to generate single-component probes for optical control over Ras/Erk or PI3K signaling. We characterized these probes in mammalian cells and found that the BcLOV4-based probes can provide signaling through a large, physiologically relevant dynamic range with low basal signaling and high photosensitivity. In addition, BcLOV4-based probes are single-protein systems, eliminating the need for stoichiometric tuning of analogous multicomponent tools. Such tuning can be difficult in model organisms like Drosophila and zebrafish, in which we show that BcLOV4-based probes function well. More generally, our work adds to the growing library of BcLOV4-based optogenetic signaling tools, highlighting BcLOV4 as a modular optogenetic actuator of effector/membrane interaction to regulate signaling across biological models, including yeast, flies, zebrafish and mammalian cells.

We discovered that BcLOV4 is a temperature sensor in addition to its known role as a photosensor. Although temperature-dependence has been observed in certain photosensors and optogenetic probes, this dependence mostly manifests as decreased protein stability or photoreactivity at elevated temperatures. By contrast, BcLOV4 folds and translocates rapidly when exposed to light at all temperatures, but then, under sustained illumination, enters a long-lived inactive state and reverts to the cytoplasm at a rate that increases with both temperature and light dose (FIGS. 7A-8G and 23A-G). This behavior is consistent with a temperature-dependent photoinactivation in which, once BcLOV4 is at the membrane in its active state, elevated temperatures accelerate its transition into a state that is incompatible with membrane binding. Although the structural details of such inactivation remain unknown, light-induced oxidation and thermal denaturation during the photocycle have both been observed in blue-light photosensors and could conceivably play a role in BcLOV4 temperature inactivation.

We developed quantitative models of BcLOV4 membrane translocation and signal activation to predict activity as a function of light and temperature. We found that sustained, whole-cell illumination will result in sustained translocation only under low light exposure or low temperature. These conditions may explain why BcLOV4 inactivation was not previously noticed, as its use to date has been performed at either ≤30° C. or over short periods, necessitated only sparse illumination (˜1% duty cycle), or used subcellular regions of stimulation, which preserves unstimulated, activatable BcLOV4 outside the region of illumination.

In addition to shaping long-term activation dynamics, BcLOV4 temperature sensitivity can be leveraged to allow multiplexing of blue-light sensitive tools in single cells, allowing control of three or four distinct cell states using a single blue-light channel. Our approach is complementary to a recent report wherein distinct transcriptional targets were activated using blue light with different temporal patterns. Our method provides similar capability but at the post-translational level; for example, for the study of how multiple signals (for example, Ras and PI3K) are integrated in single cells. We note that when attempting four-state control (with Cry2), the duration of the BcLOV4-ONLY state can be altered by tuning the ability of Cry2 to form large clusters, either by changing Cry2 concentration or through the use of Cry2 variants that change its propensity for cluster formation.

Combined with previous work, our studies provide a roadmap for how to use BcLOV4-based optogenetic tools. BcLOV4 membrane recruitment can be faithfully and precisely controlled over short durations (less than ˜30 min) across temperatures but requires low temperatures or sparse illumination for sustained (>30 min) stimulation. Specific BcLOV4 translocation dynamics over a range of light and temperature conditions can be predicted using our three-state model (FIGS. 8E-G). Although our work predicts a temperature-inactivated state of BcLOV4, future studies are required to understand the molecular basis for this temperature sensitivity. Such studies will inform protein engineering efforts to modulate BcLOV4 temperature-responsiveness for enhanced optical and thermal control across biological systems.

### METHODS

Cell culture. Lenti-X HEK 293 T cells were maintained in 10% fetal bovine serum (FBS) and 1% penicillin/streptomycin (P/S) in DMEM. NIH 3T3 cells were maintained in 10% calf serum and 1% P/S in DMEM. All cells were cultured in standard cell culture incubators at 37° C. and 5% CO2. Drosophila S2 cells were maintained in Schneider's Drosophila medium with 10% heat-inactivated FBS at room temperature. All cell lines were purchased commercially (Lenti-X HEK 293 T cells: Takarabio, catalog number 632180; NIH 3T3: ATCC, catalog number CRL-1658; S2: ThermoFisher Scientific, catalog number R69007). Cell lines were not verified after purchase. Cells were not cultured in proximity to commonly misidentified cell lines.

Plasmid design and assembly. Constructs for stable transduction in mammalian cells were cloned into the pHR lentiviral backbone with an spleen focus forming virus (SFFV) promoter driving the gene of interest. The pHR backbone was linearized using MluI and NotI restriction sites. BcLOV4, ILID, BFP, SOScat and iSH coding DNA fragments were generated via PCR and inserted into the pHR backbone via HiFi cloning mix (New England Biolabs). For expression in Drosophila S2 cells, BcLOV-mCherry, BcLOV-iSH and BcLOV-SOS_{cat} were amplified and inserted into the pbphi-nanos promoter-α Tubulin 3′-untranslated region vector44 between the NheI and BamHI restriction sites. The resulting vectors were digested with NotI and XhoI to replace the nanos protomer with the metallothionein promoter45, which was synthesized by gBlocks gene fragments (Integrated DNA Technologies). The metallothionein promoter permits inducible expression in the presence of heavy metals, for example, copper. For zebrafish mRNA expression experiments, BcLOV-mCherry, BcLOV-SOScat and ERK-KTR-BFP were amplified with primers containing att sites for Gateway cloning. PCR amplicons were transferred into pDONR221 plasmids and sequence verified. Gateway cloning was used to transfer each insert into pCSDest plasmids.

Plasmid transfection. HEK 293 T cells were transfected using the following calcium phosphate method: per 1 ml of media of the cell culture to be transfected, 50 μl of 2×HEPES-buffered saline (HeBS)28,29, 1 μg of each DNA construct and H2O up to 94 μl was mixed. Six microliters of 2.5 mM CaCl2 was added after mixing of the initial components, incubated for 1 min 45 s at room temperature and added directly to the cell culture. S2 cells were transfected with Lipofectamine 3000 reagent (ThermoFisher Scientific) following the manufacturer's protocol. The transfection mixture contained 10 ng μl−1 of DNA, 1.5% Lipofectamine 3000 reagent and 2% P3000 reagent, and was brought up to volume with Opti-MEM (ThermoFisher Scientific). The transfection mix was incubated for 15 min at room temperature and was then added directly to the S2 cells. One hundred microliters of transfection mix per 1 ml of cell culture media was used. The transfected cells were imaged 72 h after the transfection (24 h after promoter induction).

Lentiviral packaging and cell line generation. Lentivirus was packaged by cotransfecting the pHR transfer vector, pCMV-dR8.91 (Addgene, catalog number 12263), and pMD2.G (Addgene, catalog number 12259) into Lenti-X HEK 293 T cells. Briefly, cells were seeded one day before transfection at a concentration of 350,000 cells ml−1 in a six-well plate. Plasmids were transfected using the calcium phosphate method. Media was removed one day post transfection and replaced with fresh media. Two days post transfection, media containing virus was collected and centrifuged at 800 g for 3 min. The supernatant was passed through a 0.45-μm filter. Five hundred microliters of filtered virus solution was added to 100,000 NIH 3T3 cells seeded in a six-well plate. Cells were expanded over multiple passages, and successfully transduced cells were enriched through fluorescence-activated cell sorting (BD FACS Aria II) (see gating strategy in FIG. 29).

Zebrafish maintenance and mRNA injection. For messenger RNA (mRNA) generation, pCSDest BcLOV-SOScat, pCSDest BcLOV-mCherry and pCSDest ERK-KTR-BFP were digested with NotI. mRNA was generated using the SP6 mMessage Machine kit (Invitrogen) according to the manufacturer's specifications. 400 pg of BcLOV-SOScat or BcLOV-mCherry were injected. For the KTR construct, we injected 100 pg. For double injections, mRNAs were mixed before injection. Embryos were derived by natural spawning in the morning of injection and injected with the desired construct(s). Imaging was performed at 24 h post fertilization after embedding the embryos in 1% low melting point agarose in a glass bottom dish. Wild-type fish of the AB strain were used for experiments at the indicated time points of development. The sex of the animals cannot be determined at the embryonic stage.

Preparation of cells for plate-based experiments. Plates (with 96 or 384 wells) were seeded with cells, as previously described27. Briefly, wells were coated with 50 μl of MilliporeSigma Chemicon Human Plasma Fibronectin Purified Protein fibronectin solution diluted 100 times in PBS and were incubated at 37° C. for 30 min. NIH 3T3 cells were seeded in a 96- or 384-well format at a density of 3,500 or 1,000 cells per well in 100 or 50 μl, respectively and were spun down at 100g for 1 min. After 24 h, cells were starved by performing seven 80% washes with starvation media (DMEM+1% P/S). Experiments were performed after 3 h of starvation.

Optogenetic stimulation. The optoPlate-96 was used for optogenetic stimulation of individual wells in microwell plates. A single-color optoPlate was configured with two blue LEDs for maximum dynamic range of blue-light intensity. The Arduino IDE (v.1.8) was used to program the Arduino Micro found on the optoPlate-96. A low-profile (9 mm tall) well plate adapter was used for experiments in which we simultaneously stimulated and modulated sample temperature. A tall (20 mm) adapter was used for experiments in 384-well plates, as recommended. Stimulation time courses were performed by assigning time points to individual wells. Wells corresponding to different time points were started sequentially, such that all wells could be fixed simultaneously at the end of each experiment. For live-cell imaging experiments, the 488 nm laser was used to stimulate BcLOV4 membrane translocation.

Temperature-controlled optoPlate experiments. Control of sample temperature leveraged the fact that the optoPlate generates heat when operated under conditions that draw large amounts of current27. To independently control a sample's illumination conditions and its temperature, we designated 24 LEDs as ‘stimulation’ LEDs and repurposed the remaining 72 LEDs as ‘heater’ LEDs (for details see FIGS. 7A-H and 20A-G). Constant illumination of the 72 heater LEDs at varying intensities permitted linear and uniform control of temperature in the 24 sample wells. Experiments were performed with the heatsink fan operating at maximum speed. To perform temperature-controlled experiments, the cell culture incubator temperature was reduced to 25 or 30° C. and optoPlate heating was used to increase temperature according to the relationship described in FIG. 7F. Sample plates were first equilibrated to the lower incubator temperature for 2 h, and then equilibrated on the optoPlate to achieve the desired increased temperature for 1.5 h before the illumination program began.

Immunofluorescence staining. Immediately following completion of a stimulation protocol, 16% paraformaldehyde was added to each well to a final concentration of 4%, and cells were incubated in paraformaldehyde in the dark for 10 min. Cells were then permeabilized with 100 or 50 μl (for 96- or 384-well plates) with PBS+0.1% Triton X-100 for 10 min. Cells were then further permeabilized with ice-cold methanol for 10 min. After permeabilization, cells were blocked with 1% BSA at room temperature for 30 min. Primary antibody was diluted in PBS+1% BSA according to the manufacturer's recommendation for immunofluorescence (phospho-p44/42 MAPK (Erk1/2) (Thr202/Tyr204), Cell Signaling, catalog number 4370, 1:400 dilution; phospho-Akt (Ser473), Cell Signaling Technologies, catalog number 9271, 1:800 dilution). Plates (96 or 384 wells) were incubated with 50 or 25 μl of antibody dilution for 2 h at room temperature. Samples were incubated at room temperature in primary antibody for 2 h, after which primary antibody was removed and samples underwent five washes in PBS+0.1% TWEEN-20 (PBS-T). Cells were then incubated with secondary antibody (Jackson Immunoresearch Alexa Fluor 488 AffiniPure goat anti-rabbit IgG (H+L)) and 4,6-diamidino-2-phenylindole (DAPI; ThermoFisher Scientific, catalog number D1306, 300 nM) in PBS-T+0.1% BSA for 1 h at room temperature. Secondary antibody was removed, and samples underwent five washes with PBS-T. Samples were imaged in PBS-T.

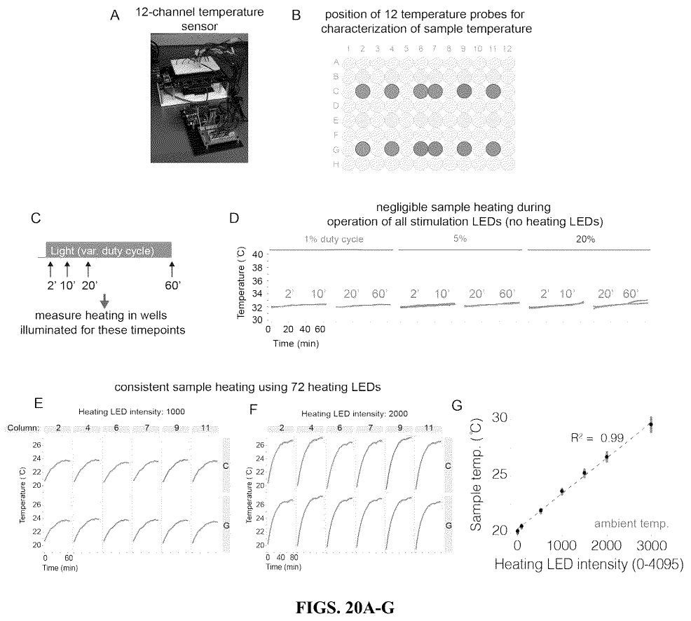

Imaging. Live-cell imaging. Live-cell imaging was performed using a Nikon Ti2E microscope equipped with a Yokagawa CSU-WI spinning disk, 405/488/561/640 nm laser lines, an sCMOS camera (Photometrics), a motorized stage and an environmental chamber (Okolabs). HEK 293 T cells expressing BcLOV-mCherry were imaged with a ×20 objective at variable temperatures and 5% CO2. Cells were incubated at the desired temperature for 2 h before imaging to ensure cells equilibrated at the desired temperature. Temperatures were verified by using the temperature sensor shown in FIG. 20A. The temperature probes were submerged in PBS in the wells of a 96-well plate, and the probe/plate apparatus was placed on the microscope stage inside the environmental chamber to record temperatures during imaging. BcLOV4 was stimulated using a 488 nm laser. Zebrafish and Drosophila S2 cells were imaged at room temperature using a ×40 oil immersion objective.

High-content imaging. Fixed samples were imaged using a Nikon Ti2E epifluorescence microscope equipped with DAPI/FITC/Texas Red/Cy5 filter cubes, a SOLA SEII 365 LED light source and motorized stage. High-content imaging was performed using the Nikon Elements AR software. Image focus was ensured using image-based focusing in the DAPI channel.

Image processing and analysis. Immunofluorescence quantification. Images were processed using Cell Profiler. Cells were segmented using the DAPI channel, and cytoplasm was identified using a five-pixel ring around the nucleus. Nuclear and cytoplasmic fluorescence values were then exported and analyzed using R (cran.r-project.org) and R-Studio (rstudio.com). Data was processed and visualized using the dplyr and ggplot2 packages. For FIGS. 7G-H, exponential decay functions of the form a×e^{-b×x} were fit to data points in each condition to visualize the rate of decay in signaling. Curves were fit using the MATLAB R2020a cftool program.

Membrane recruitment. Membrane localization was quantified using the ilastik machine learning software50. Briefly, ilastik was used to identify pixels that correspond to the plasma membrane based on user annotations of images of cells that expressed infrared fluorescent protein (iRFP)-CAAX protein, which localizes to the plasma membrane. The resulting image masks were imported into Cell Profiler and were used to quantify the amount of BcLOV4 membrane localization within the same frame. Total BcLOV4, membrane-localized BcLOV4 and total iRFP-CAAX intensity was recorded and further processed in R. Bleaching was corrected by dividing the total intensity of masked mCherry images by the total intensity of mCherry in the original unmasked image. This method assumed that loss of fluorescence was not due to degradation, which we empirically confirmed (FIGS. 24A-C). Zebrafish and Drosophila BcLOV4 membrane recruitment in FIGS. 10A-H was performed by manually comparing the pixel intensity of membrane and cytoplasmic intensities in ten or more cells.

Modeling. The three-parameter model found in FIG. 8A-G was based on the following observations: (1) The rate of transition from the TI to the dark state can be approximated as 0 because BcLOV4 inactivation is effectively irreversible (FIG. 7C). (2) The rate of BcLOV4 transition from the dark state directly to the TI state can be approximated as 0 because BcLOV4 could be strongly activated even when preincubated for 2 h before stimulation across a wide range of experimental temperatures, with no obvious correlation between temperature and signal strength (FIGS. 21A-E). (3) The rate of BcLOV4 transition from the TI state to the lit state can be approximated as 0 because temperature-inactivated membrane recruitment and signal activation decay to zero, whereas a nonzero reversion to the lit state would result in a nonzero equilibrium between lit and inactivated state.

Under these assumptions, the following equations were used to model BcLOV4 activity. These equations allow only for bidirectional BcLOV4 movement between the dark and lit states and irreversible movement from the lit state to the TI state.

$$\frac{dD}{dt} = - D \times {k}_{1} + L \times {k}_{2} \qquad ( 1 )$$

$$\frac{dL}{dt} = D \times {k}_{1} - L \times {k}_{2} - TI \times {k}_{3} \qquad ( 2 )$$

$$\frac{dTI}{dt} = L \times {k}_{3} \qquad ( 3 )$$

where D=Dark State BcLOV4, L=Lit State BcLOV4, TI=Temperature Inactivated State BcLOV4, k₁=Dark to Lit state transition rate, k₂=Lit to Dark state transition rate, and k₃=Lit to Temperature Inactivated state transition rate. This system of equations was implemented in MATLAB and was solved numerically using the Euler method. The rate constants k₁, k₂ and k₃ were found by fitting the model to live-cell imaging data of BcLOV4 membrane translocation through minimization of mean squared error (FIG. 8F). Fitting was performed using a custom script that iteratively calculated vertical R-squared across a coarse-grained range of parameter values, and then testing a more finely resolved set of values centered on the best fitting value chosen from the previous iteration. Under the assumption that:

k₁, k₂»k₃ (4)

k₁ and k₂ were found by setting k₃=0 and fitting observed BcLOV4 translocation kinetics over short (1 min) periods, yielding the following rate constants:

k₁=60±5 min⁻¹, k₂=1.5±0.2 min⁻¹

k₃ was then determined by keeping k₁ and k₂ constant and fitting k₃ to observed decay rates at each temperature.

The transfer function model of SOS_{cat}-to-ppErk transmission was implemented using the image processing toolbox in MATLAB. Based on previous work, we hypothesized that the transfer function could be modeled as a 1° or 2° LPF:

$$1 ° LPF : H ( s ) = \frac{{ω}_{0}}{s + {ω}_{0}} \qquad ( 5 )$$

$$2 ° LPF : H ( s ) = \frac{{ω}_{0}^{2}}{{( s + {ω}_{0} )}^{2}} \qquad ( 6 )$$

where H(s) is the ratio between system input and output, s is the complex variable (frequency) and ω₀ is the cutoff frequency. To discriminate between these potential models, we measured ppErk in cells when stimulated by a dynamic 2 min ON/2 min OFF pulse train of light, which corresponds to a frequency that should be ˜90% suppressed by the second-order LPF, but substantially less suppressed (˜40%) by a first-order LPF (FIG. 9B). We integrated the 1° or 2° LPF models into our model of BcLOV4 translocation, and we fit these integrated models to the data as described above. We found that dynamic Erk stimulation through BcLOV4 translocation was best described by a 1° LPF with a 2-mHz cutoff frequency.

For all experiments related to modeling, illumination duty cycle was used to modulate the intensity of BcLOV4 stimulation. Duty cycle parameters were limited to patterns where the OFF period was less than ˜1 min to ensure that BcLOV4 membrane recruitment was maintained at intermediate levels, as determined by its measured inactivation kinetics 15.

Multiplexing experiments. HEK 293 T cells were seeded in a 96-well plate and were cotransfected with 100 ng each of the following plasmids. BcLOV/iLID multiplexing: BcLOV-mCherry, sspb-GFP-P2A-iLID-CAAX and iRFP-CAAX; BcLOV/Cry2 multiplexing: BcLOV-GFP, Cry2(PHR)-mCherry13. Images were acquired 24 h post transfection using confocal microscopy. For BcLOV/iLID multiplexing, light stimulation was performed at 37° C. using 1 s of blue light (1.45 W cm−2) every 30 s for 10 min in the presence or absence of prior

BcLOV inactivation. Inactivation was achieved using these same light settings for 1 h. For BcLOV/Cry2 multiplexing, ‘short light’ (10 min) and ‘long light’ (45 min) exposure was achieved using 100 ms of light (1.45 W cm−2) every 30 s at 30° C. BcLOV inactivation was achieved using 1 s of blue light (1.45 W cm−2) every 30 s for 45 min. The Cry2-ONLY state was imaged after 45 min of light inactivation.

### Example 2

Further to the discussion of light and temperature sensitivity in Example 1, this Example discusses the change in temperature activation of lit-state BcLOV4 proteins.

Modulation of strength of BcLOV-T membrane association with polybasic domains. Appending short polybasic domains to BcLOV-T can strengthen the magnitude of membrane association. One such domain is from the STIM1 protein (DSSPGRKKFPLKIFKKPLKK). Compare membrane association at either temperature between the panel A(-STIM) and panel B (+STIM). Tandem STIM domains (Panel C, Stim2x, DSSPGRKKFPLKIFKKPLKKDSSPGRKKFPLKIFKKPLKK) and a polybasic domain from the Rit protein (Panel D, MEKKSKPKNSVWKRLKSPFRKKKDSVT) give progressively stronger membrane association.

Modulation of temperature switchpoint of BcLOV-T membrane association. The mutation of the C292 amino acid to other residues can tune the thermal switch-point, The images show steady-state membrane association at either 37 C or 40 C. A) BcLOV-T(Q355N) can be modulated at lower temperatures, but does not show a difference in membrane association between 37C and 40C. B) A C292A point mutation allows membrane binding at 37 C but not 40 C. C) A C292P point mutation allows stronger membrane association at 37 C than the C292A mutation, but membrane is still eliminated at 40 C. We also screened the other 18 amino acids at 293 position and successfully generated a library of BcLOVT variants with different switching temperatures for membrane translocation. D) We also found that appending short polybasic domains to BcLOV-T can strengthen the magnitude of membrane association. One such domain is from the STIM1 protein (DSSPGRKKFPLKIFKKPLKK). Compare membrane association at either temperature between panels B (−STIM) and panel D (+STIM). The Stim2x and Rit polybasic domains also increase strength of membrane binding of the BcLOV-T Q355N mutant.

### Example 3

### A Temperature-Inducible Protein Module for Control of Mammalian Cell Fate

Inducible protein switches are used throughout the biosciences to allow on-demand control of proteins in response to chemical or optical inputs. However, these inducers either cannot be controlled with precision in space and time or cannot be applied in optically dense settings, limiting their application in tissues and organisms. Here we introduce a protein module whose active state can be reversibly toggled with a small change in temperature, a stimulus that is both penetrant and dynamic. This protein, called Melt (Membrane localization through temperature), exists as a monomer in the cytoplasm at elevated temperatures but both oligomerizes and translocates to the plasma membrane when temperature is lowered. Using custom devices for rapid and high-throughput temperature control during live-cell microscopy, we find that the original Melt variant fully switches states between 28-32° C., and state changes can be observed within minutes of temperature changes. Melt was highly modular, permitting thermal control over diverse intracellular processes including signaling, proteolysis, and nuclear shuttling through straightforward end-to-end fusions with no further engineering. Melt was also highly tunable, giving rise to a library of Melt variants with switch point temperatures ranging from 30-40° C. The variants with higher switch points allowed control of molecular circuits between 37° C.-41° C., a well-tolerated range for mammalian cells. Finally, Melt could thermally regulate important cell decisions over this range, including cytoskeletal rearrangement and apoptosis. Thus Melt represents a versatile thermogenetic module that provides straightforward, temperature-based, real-time control of mammalian cells with broad potential for biotechnology and biomedicine.

### Main Text:

Inducible proteins provide a wealth of strategies for on-demand, remote control of cell behavior, for example using chemicals or light as inputs. These inputs trigger protein conformational changes that can regulate a vast array of downstream protein and cell behaviors in a modular manner. While chemical control requires delivery of a small molecule, light can be applied remotely and offers further benefits for precision in both space and time, as well as low cost of the inducer. There is tremendous potential to extend these benefits into more complex settings including in 3D cell and tissue models, in patients for control of cell therapy, or in dense bioreactors for bioproduction. However, optical control is limited in these more opaque settings because visible light cannot penetrate, for example scattering within millimeters of entering human tissue.^{1,2} There is thus a need for alternative inducer strategies that couple the penetration of chemical induction with the spatiotemporal precision of optogenetics.

Temperature has gained recent interest as a dynamic and penetrant inducer.³⁻⁶ Unlike light, temperature can be regulated tens of cm deep within tissue with sub-millimeter-scale precision using technologies like focused ultrasound that are already used in the clinic.⁷ Furthermore, unlike either chemical- or light-induction, thermal-responsiveness could uniquely interface with an organism's own stimuli, setting the stage for engineered biological systems that autonomously detect and respond to physiological temperature cues, for example fevers or inflammation.

The widespread adoption of chemo- and opto-genetic proteins was enabled by the identification protein domains that undergo stereotyped and consistent changes in response to small molecules or light. However, remarkably few analogous temperature-sensing modules have been described. Endogenous heat shock promoters have been used for thermal control of transcription, including to induce tumor clearance by engineered cells.^{4.8.9} However heat shock promoters can respond to non-thermal stimuli,¹⁰⁻¹² and thermal response profiles cannot be readily tuned because they depend on the cell's repertoire of heat shock factor proteins. Moreover, many desirable cell behaviors (e.g. migration, proliferation, survival/death) cannot be easily controlled at the transcriptional level. At the post-translational level, temperature-sensitive (Ts) mutants are protein variants that denature at elevated temperatures.¹³⁻¹⁵ However, Ts mutations are generally not modular or reversible and must be laboriously validated for each individual target. The TlpA protein from Salmonella forms thermolabile dimers¹⁶ and underlies existing thermosensitive engineered proteins, including a temperature-controlled dimerization module.¹⁷ However TlpA-based dimers are large (˜600-700 amino acids in combined size), and may be limited by the need for stoichiometric tuning between the two components. The identification of distinct temperature-responsive proteins, especially with functions beyond dimerization, is critical for broad development and application of thermogenetic approaches.

Here we introduce a unique thermoresponsive protein module called Melt (Membrane localization using temperature), which we derived from the naturally light- and temperature-sensitive BcLOV4 protein 18. Melt is a single protein that clusters and binds the plasma membrane at low temperatures but dissociates and declusters upon heating. Using live-cell imaging coupled with custom devices for precise temperature control in 96-89 well plates, we found that Melt could be toggled between these two states rapidly and reversibly, with observable membrane dissociation and recovery within 10 s of minutes. The Melt approach was highly modular, allowing thermal control of diverse processes including EGFR and Ras signaling, TEVp proteolysis, and subcellular localization through simple end-to-end fusion of the appropriate effectors. We then tuned Melt to increase its switchpoint temperature above the native 30° C. Such tuning resulted in Melt variants that operated with switch point temperatures between 30-40° C., including ones that bound the membrane at 37° C. and fully dissociated at 39° C. or 42° C., temperature ranges suitable for downstream application in mammalian tissues. These variants controlled multiple post-translational circuits between 37° C. and 42° C. and could regulate important cell-level behaviors including cytoskeletal reorganization and apoptosis. Thus Melt offers a straightforward, tunable, and broadly applicable platform for endowing thermal control across a wide range of molecular and cellular behaviors.

### RESULTS

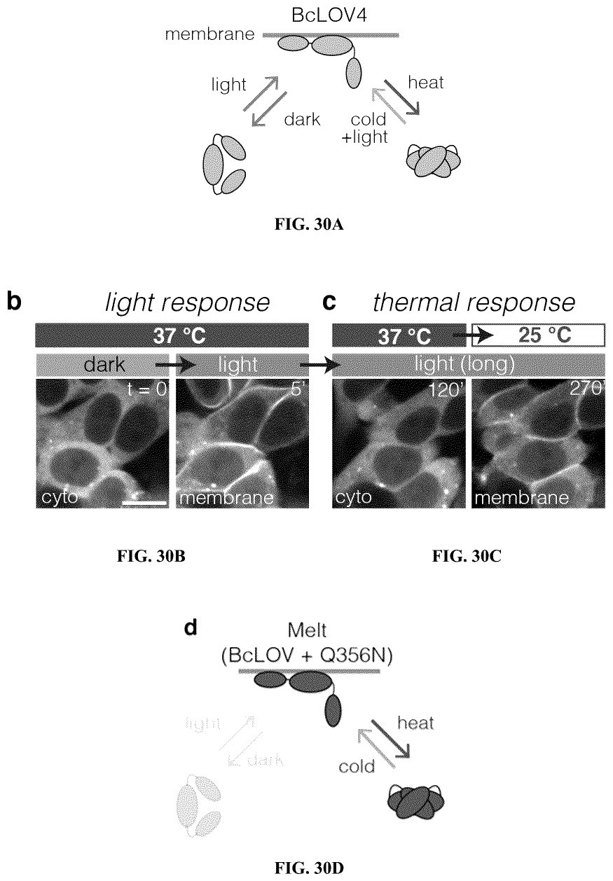

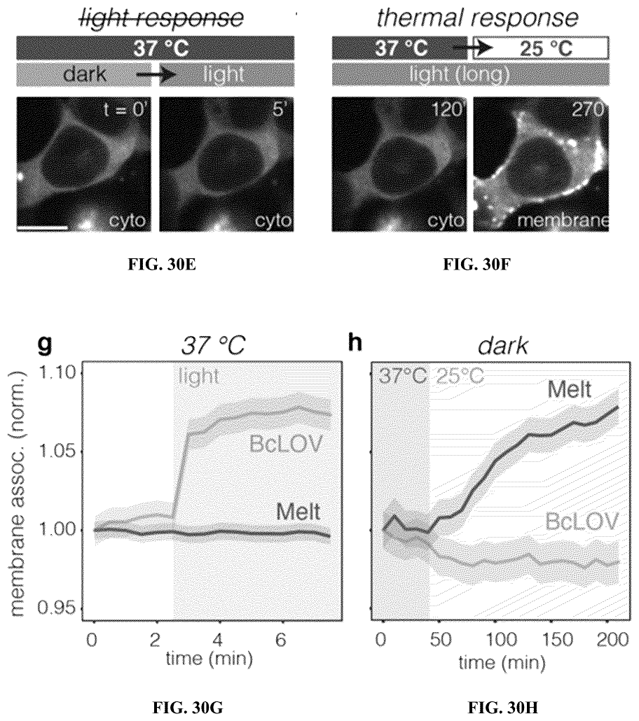

BcLOV4 is a modular optogenetic protein that natively responds to both blue light and temperature^{18,19} (FIG. 30A). Light stimulation triggers its clustering and translocation from the cytoplasm to the plasma membrane, where it binds anionic phospholipids.^{19,20} However, its persistence at the membrane requires both continued light and a permissive temperature. At temperatures above 29° C., membrane binding is transient; BcLOV4 binds but then returns to the cytoplasm (FIGS. 30A-C) at a rate that increases with temperature.¹⁸ Our previous report found that, once dissociated due to elevated temperatures, BcLOV4 remains in the cytoplasm and no longer responds to light stimuli.¹⁸ However, we found that lowering temperature below the 29° C. threshold reversed this inactivation and restored light-dependent membrane localization (FIG. 30C). Thus, temperature alone could be used to toggle the localization of BcLOV4 given the continued presence of blue light.

We sought to harness this thermal responsiveness to generate a protein actuator that responded only to temperature. We reasoned that a BcLOV4 variant with a point mutation that mimicked the “lit” state would localize to the membrane independent of light status but should retain thermal sensitivity (FIG. 30D). We thus introduced a Q355N mutation that disrupts the dark-state interaction between the Ja helix and the core of the LOV domain.^{19,21} When expressed in HEK 293T cells at 37° C., BcLOV(Q355N)-mCh appeared mostly cytoplasmic and did not respond to blue light (FIGS. 30E-G). Strikingly, shifting the temperature from 37° C. to 25° C. triggered an accumulation of the protein at the plasma membrane, where increasing accumulation was observed within minutes and continued over the next three hours (FIGS. 30D-H). Membrane localization of Melt was often accompanied by visible clustering at the membrane, consistent with our prior findings that clustering and membrane-binding are interlinked properties of BcLOV4²⁰ (FIGS. 30B-C and F). Conversely, the native photosensitive BcLOV4 did not accumulate at the membrane in response to temperature in the absence of light (FIGS. 30G-H). Thus, BcLOV4(Q355N)—henceforth referred to as Melt (Membrane localization using temperature)—is a light-insensitive protein whose subcellular localization can be regulated solely by temperature.

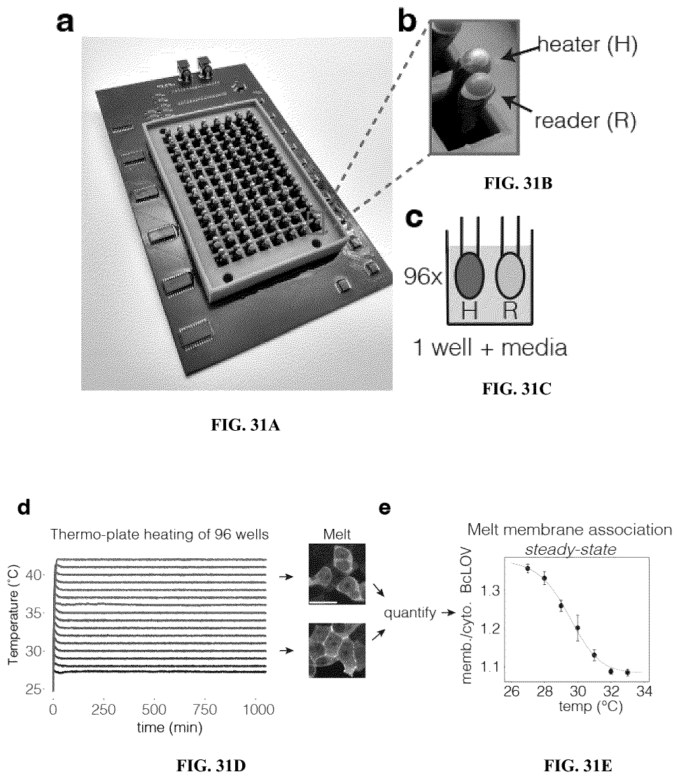

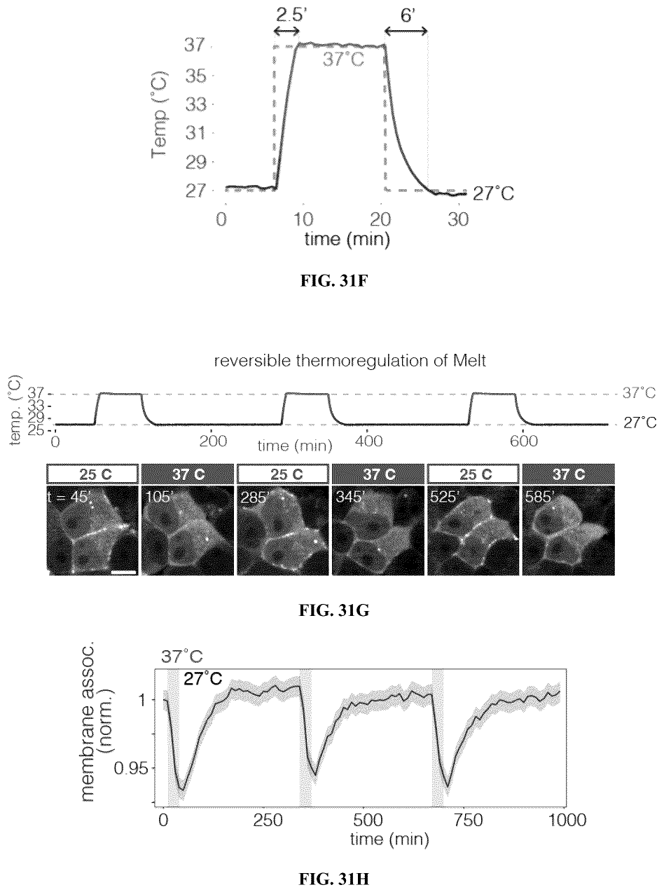

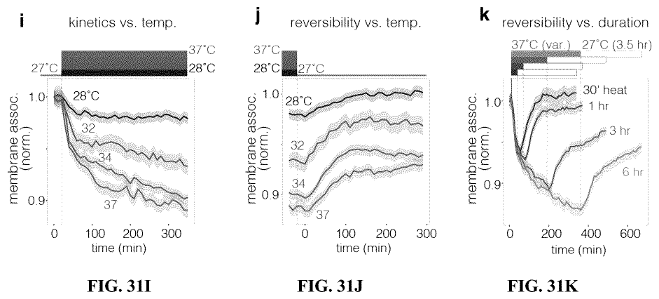

We next sought to comprehensively characterize 130 the thermal response properties of Melt, including how the amplitude and kinetics of membrane dissociation/reassociation varied with time and temperature. To systematically explore this large parameter space, we developed a device that allowed rapid, programmable heating of individual wells of 96-well plates. This device—the thermoPlate—has 96 pairs of thermistors arrayed in the format of a standard 96-well plate (FIG. 31A). One thermistor serves as a miniature immersion heater (heater, FIGS. 31B-C) that heats the medium through resistive heating, while the second acts as a thermometer (reader, FIGS. 31B-C) that measures the temperature of the medium in a well. The heater and reader implement proportional-integral-derivative (PID) feedback control, which maintains a pre-defined temperature profile over even day-long experiments (FIG. 31D). Because the thermoPlate has a thin profile and is positioned above a 96-well plate, it allows simultaneous live-cell imaging of the sample using an inverted microscope.

We first used multiplexed temperature control to measure steady-state Melt membrane association over a range of temperatures after 24 hrs of heating (FIG. 31E). Membrane association was maximal at 27° C. and minimal at 32° C., and reached 50% of this range at ˜30° C., which we assign as its switch temperature. The thermoPlate also permits observation of fast thermal response dynamics by allowing rapid temperature changes (e.g. 10 degrees heating in 2.5 mins, 10 degrees cooling in 6 minutes, FIG. 31F). By toggling temperature between 27 and 37, we could demonstrate reversible membrane binding and dissociation over multiple cycles (FIGS. 31G-H, Supplementary Movie 1). For full details on membrane binding quantification, see FIG. 35 and Methods.

We next examined the kinetics of Melt translocation to and from the membrane. Dissociation kinetics increased with higher temperatures (FIG. 311). Notably, although steady-state membrane association was unchanged above 32° C. (FIG. 31E), the rate with which Melt reached this steady state level continued to increase with temperature (note the higher decay rate at 34° C. and 37° C. relative to 32° C., (FIG. 31I). Reassociation kinetics depended on the thermal stimulation history. Samples that were stimulated at higher temperatures showed a lower degree of reversibility (FIG. 31J). Reversibility was also a function of the duration of prior stimulation. Although dissociation after 30 min of heating at 37° C. was fully reversible, longer stimulation led to smaller degrees of reversion (FIG. 31K). Collectively, these data suggest that Melt is a thermoswitch that operates tunably and reversibly within a 27-32° C. range, but whose reversibility is a function of the magnitude of its prior stimulation.

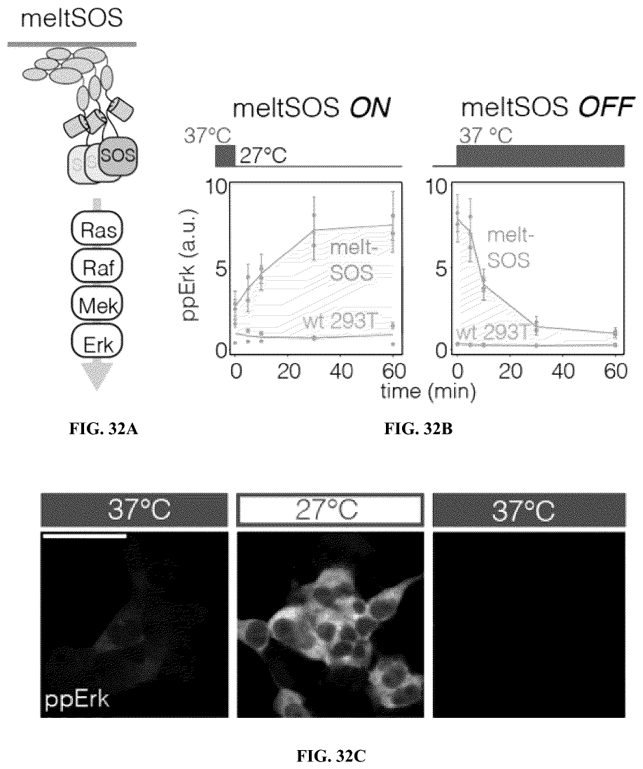

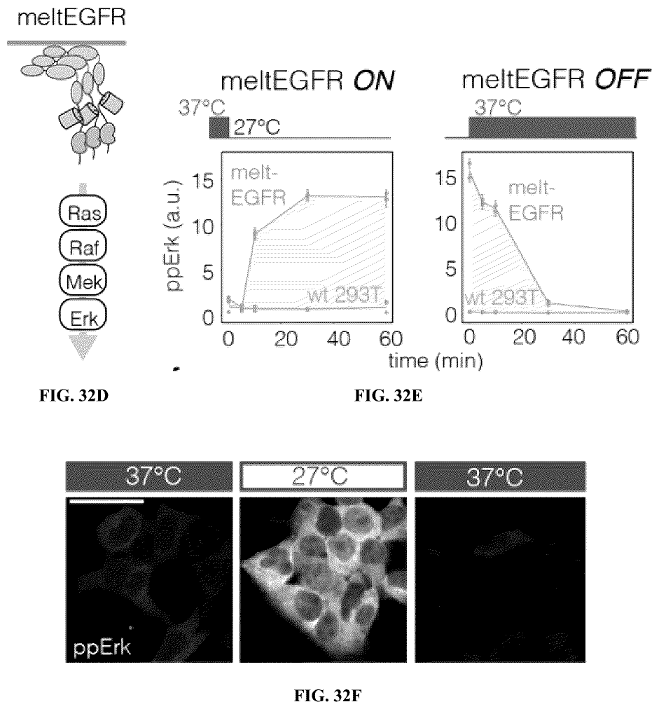

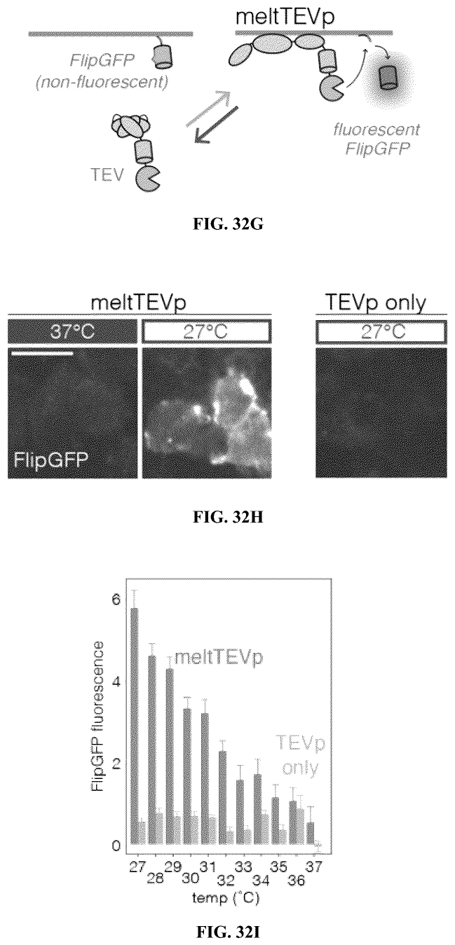

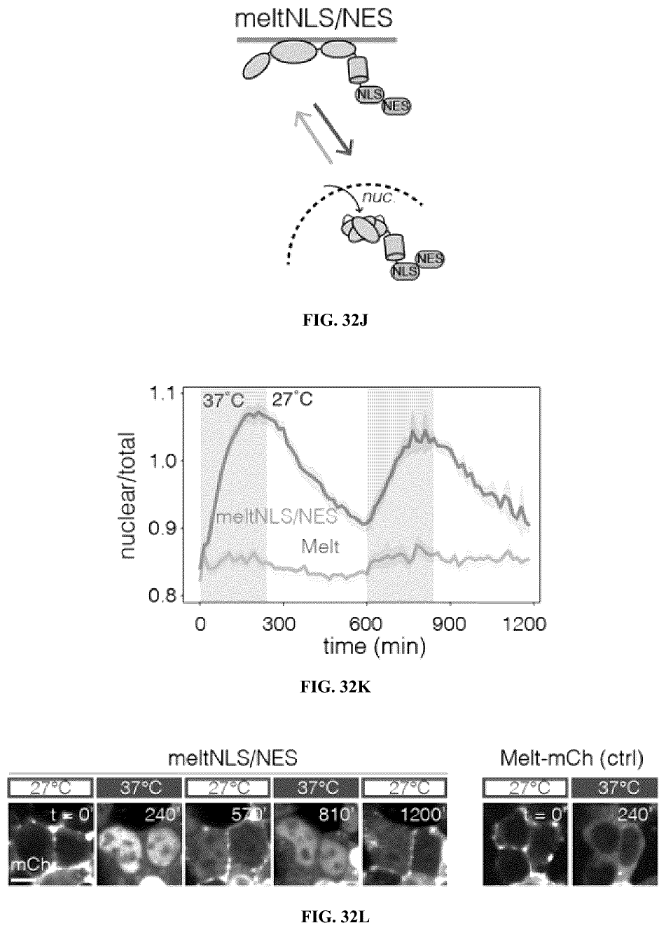

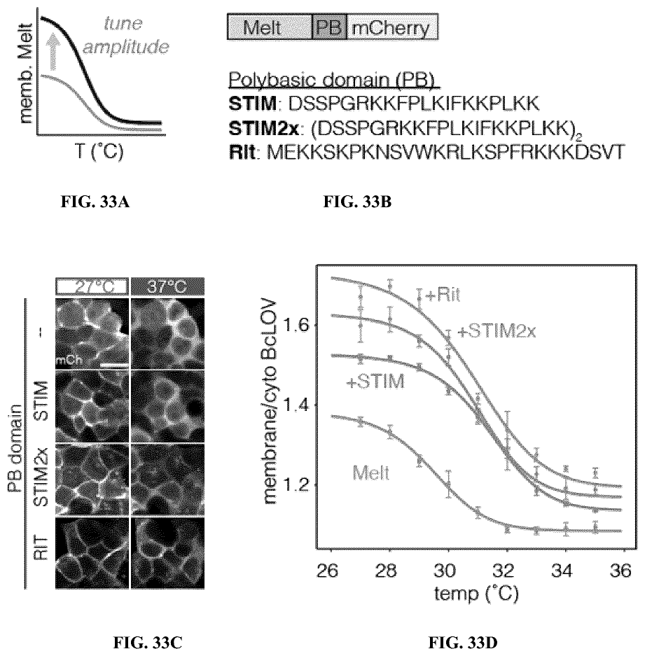

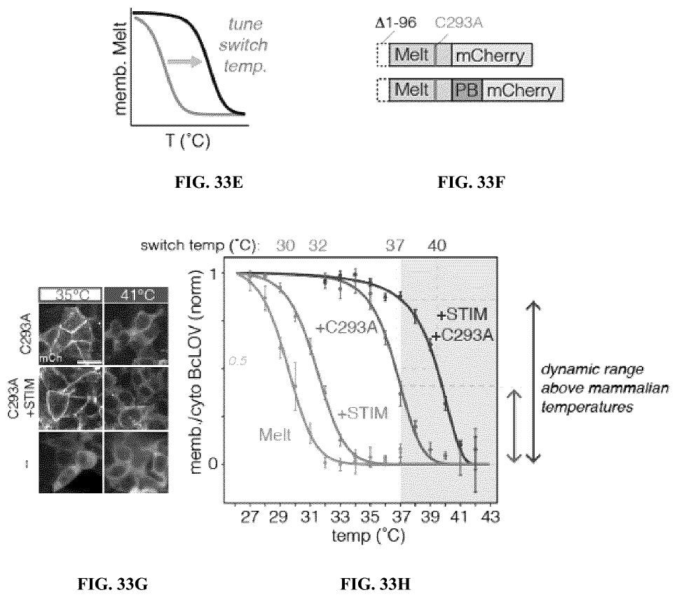

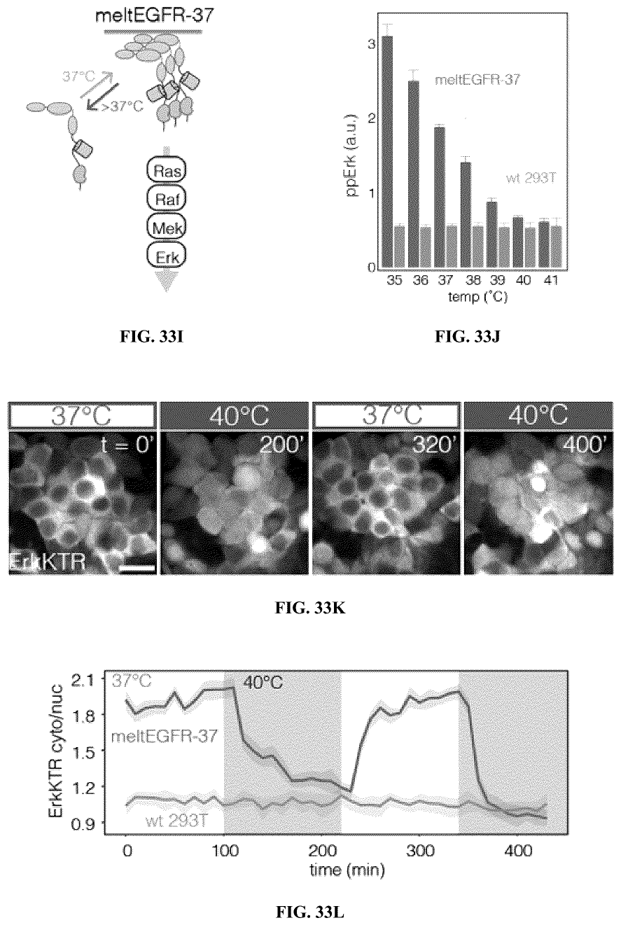

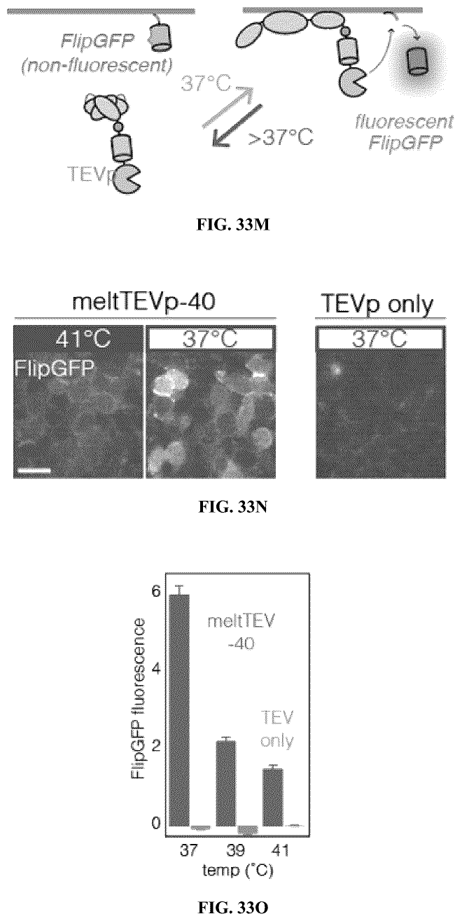

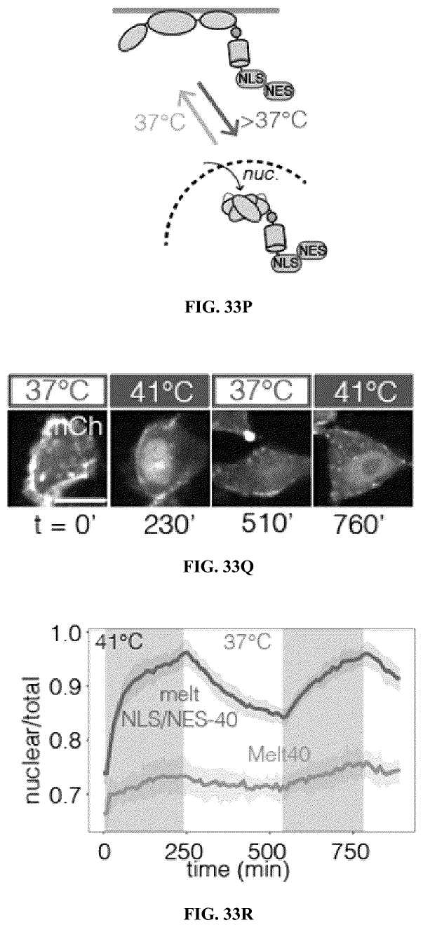

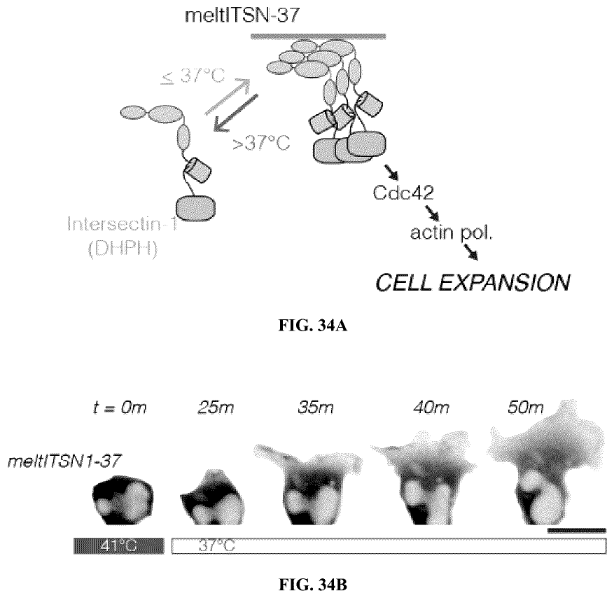

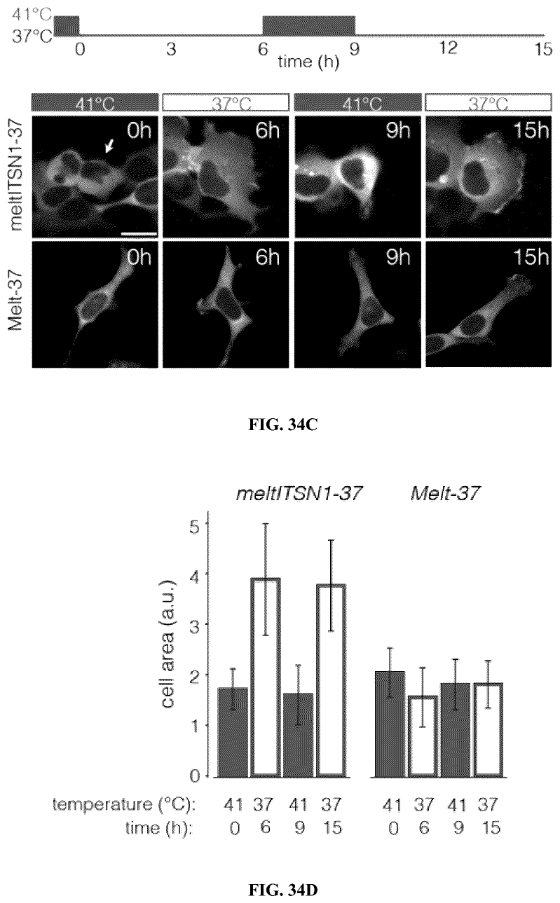

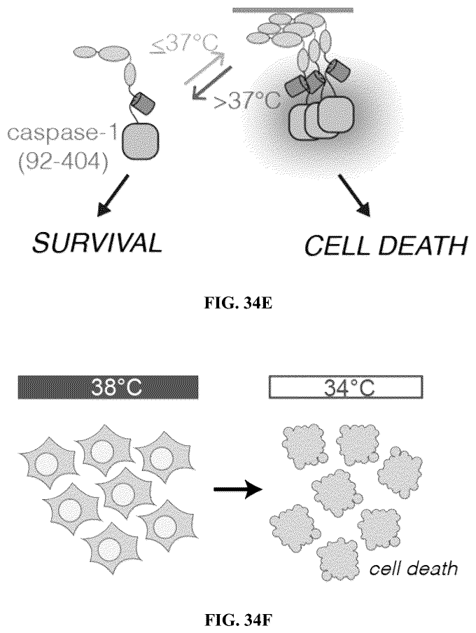

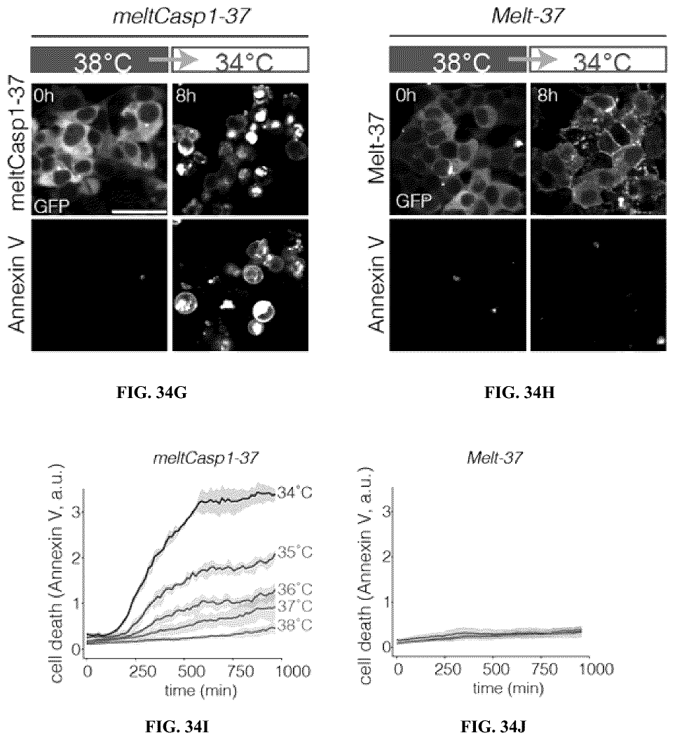

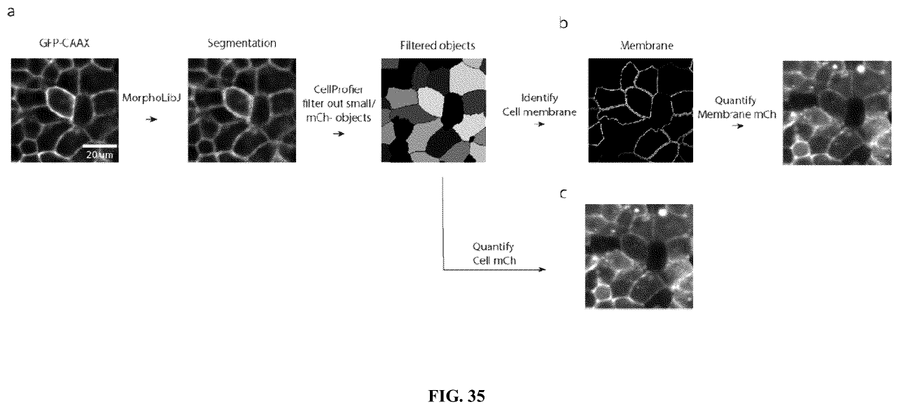

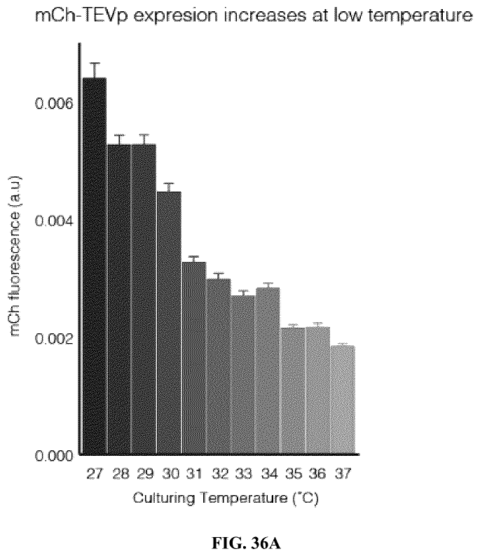

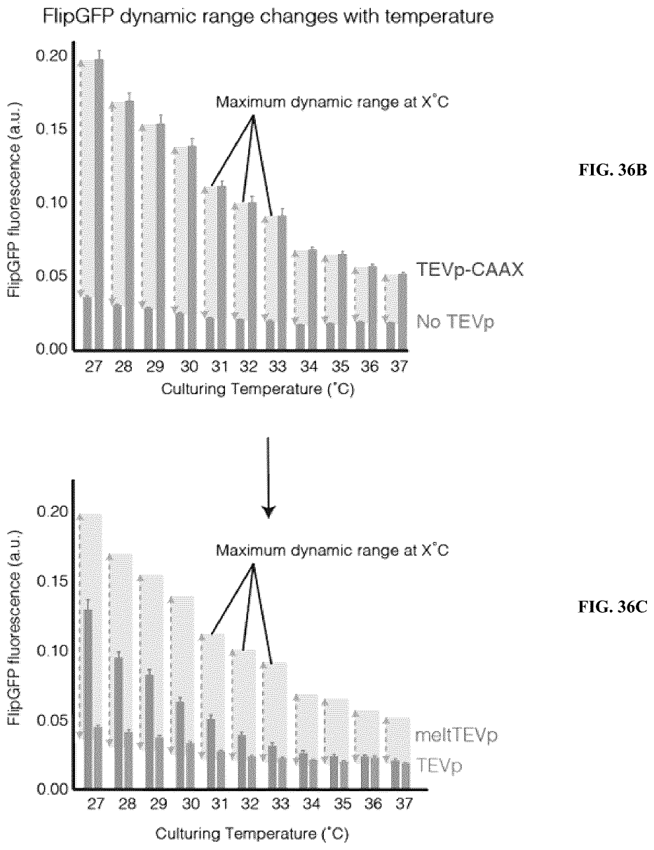

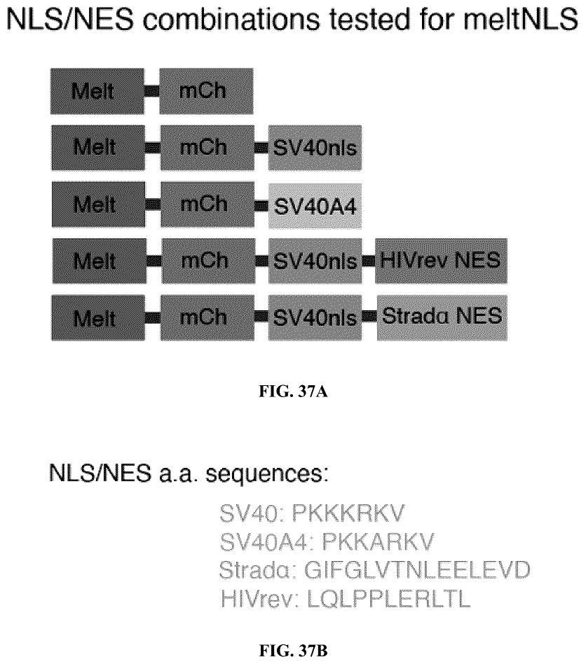

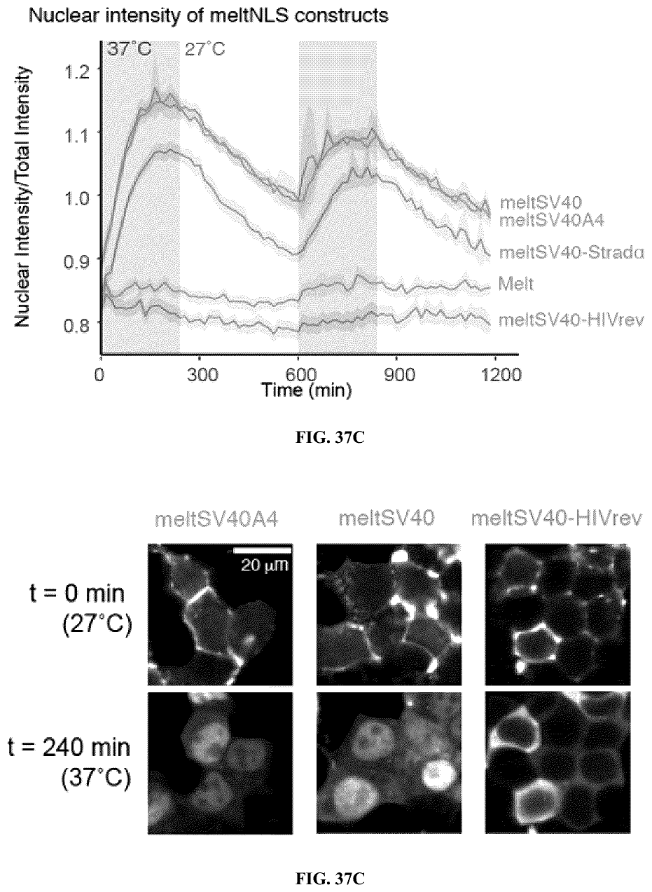

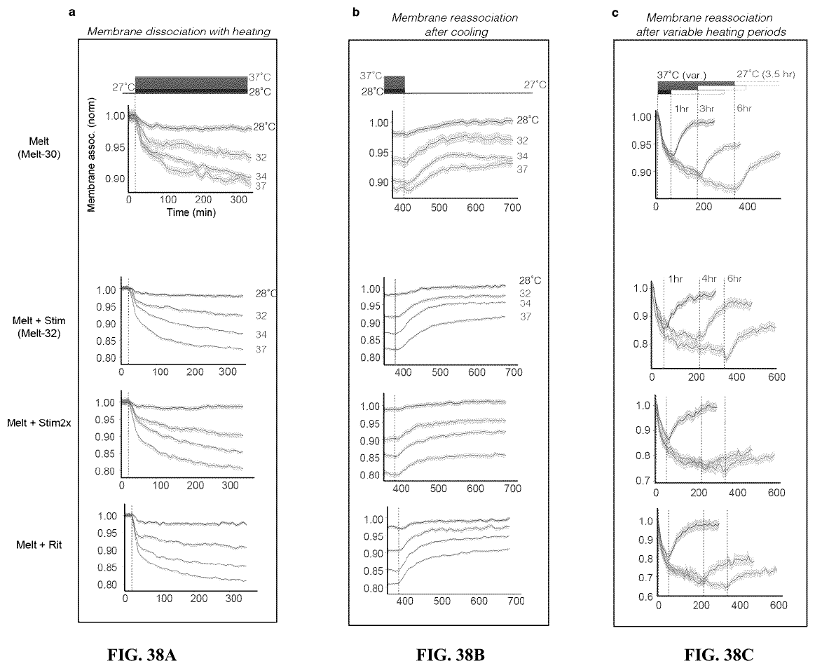

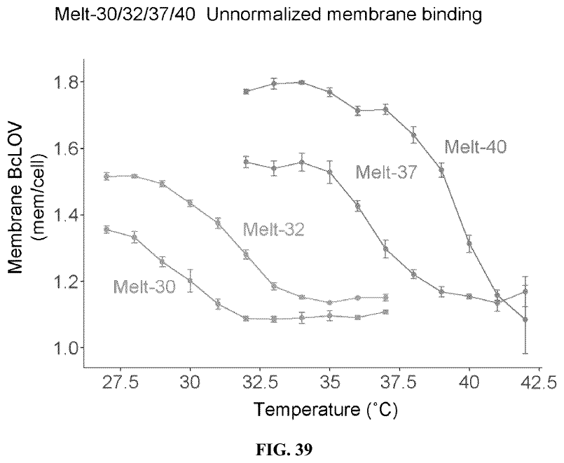

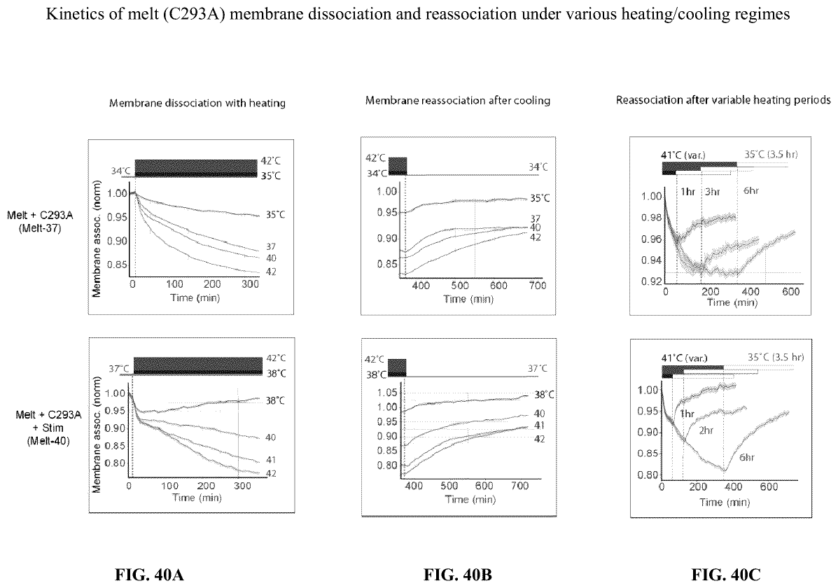

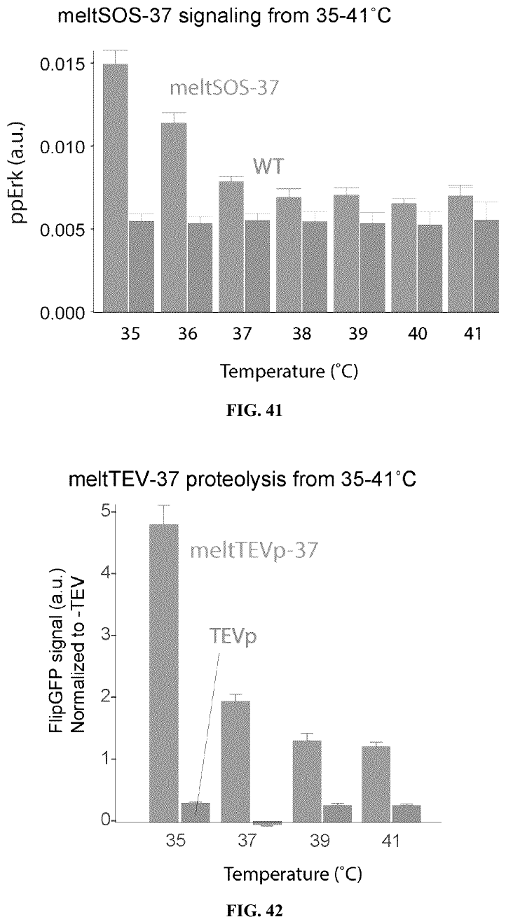

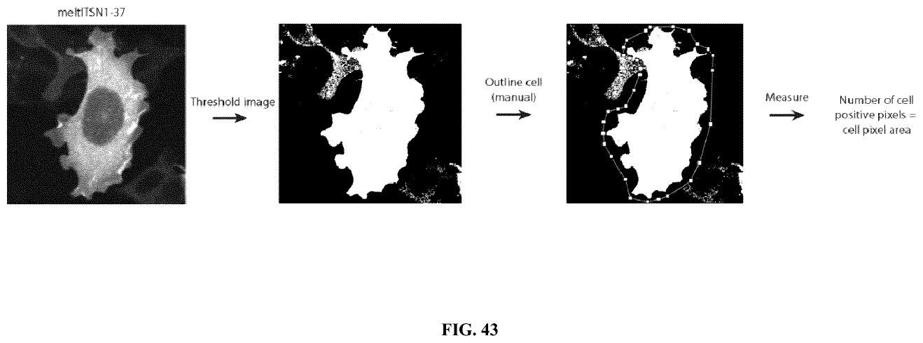

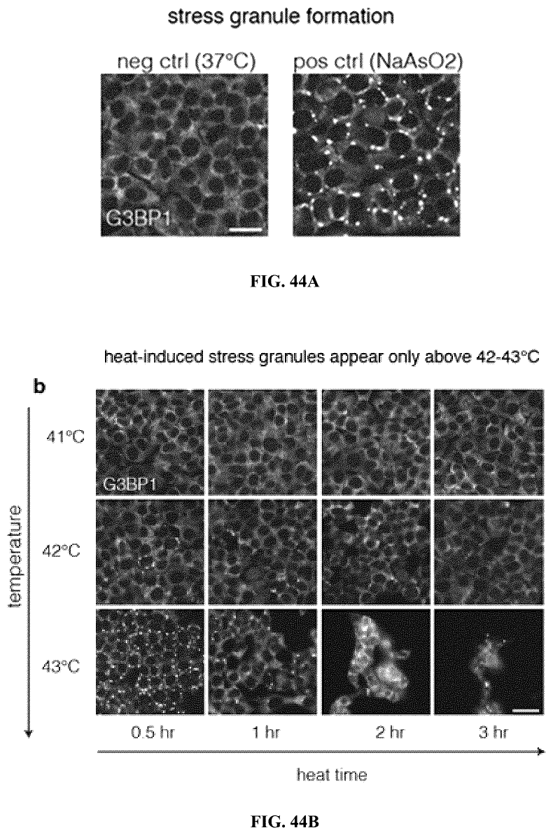

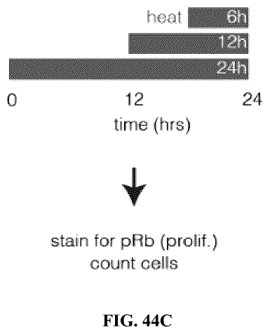

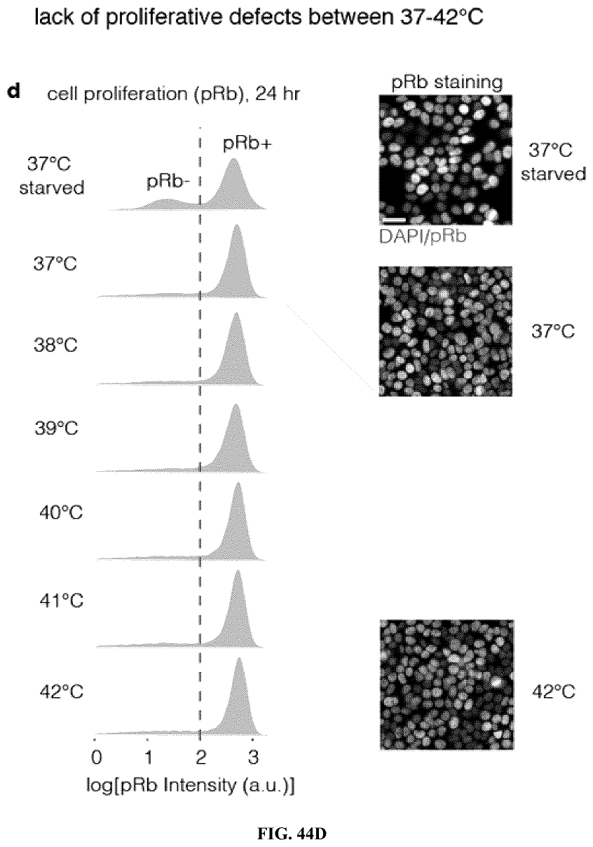

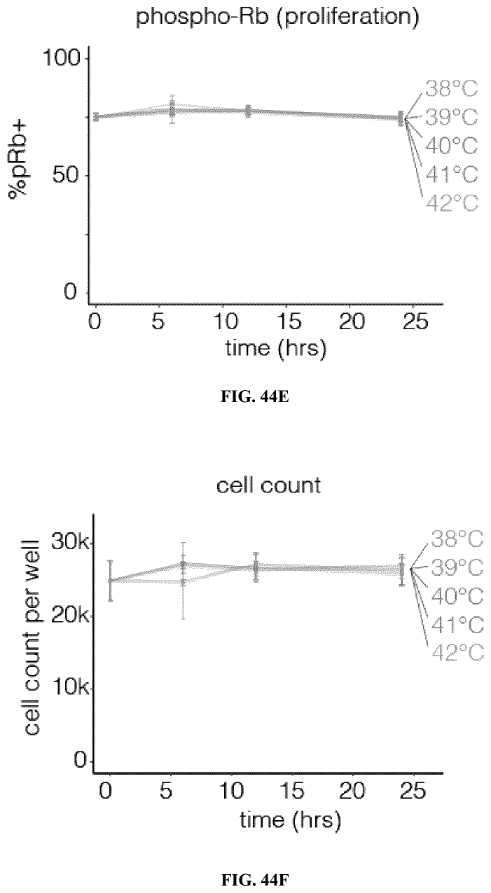

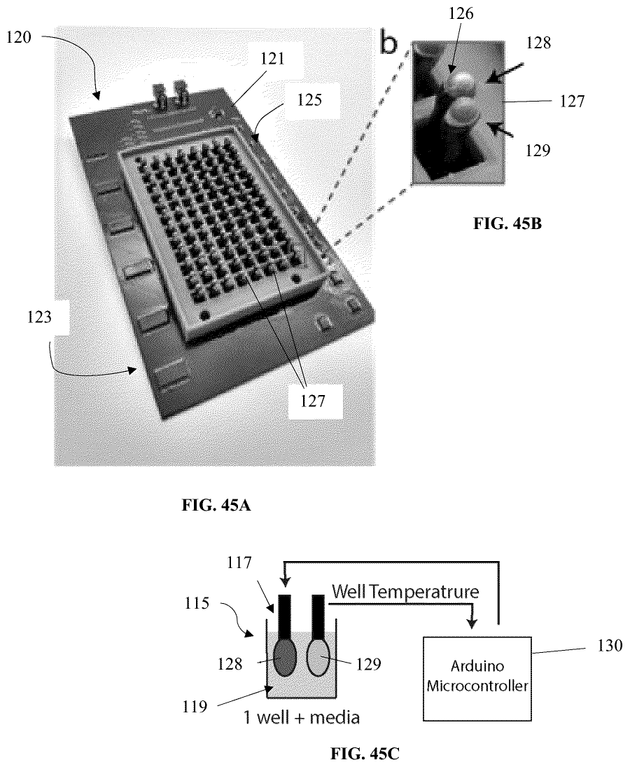

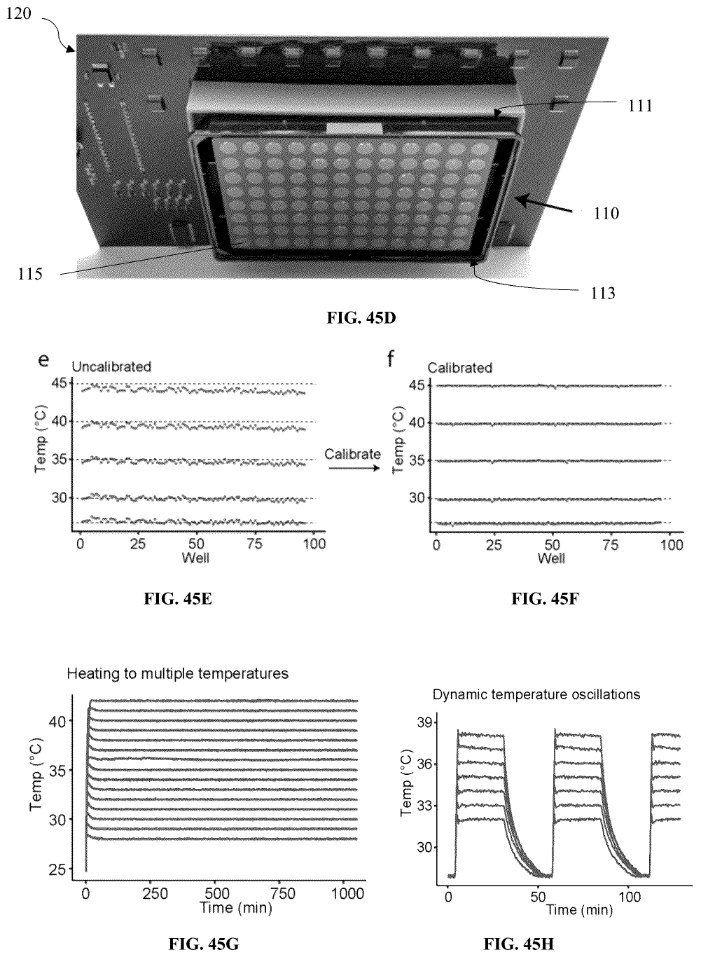

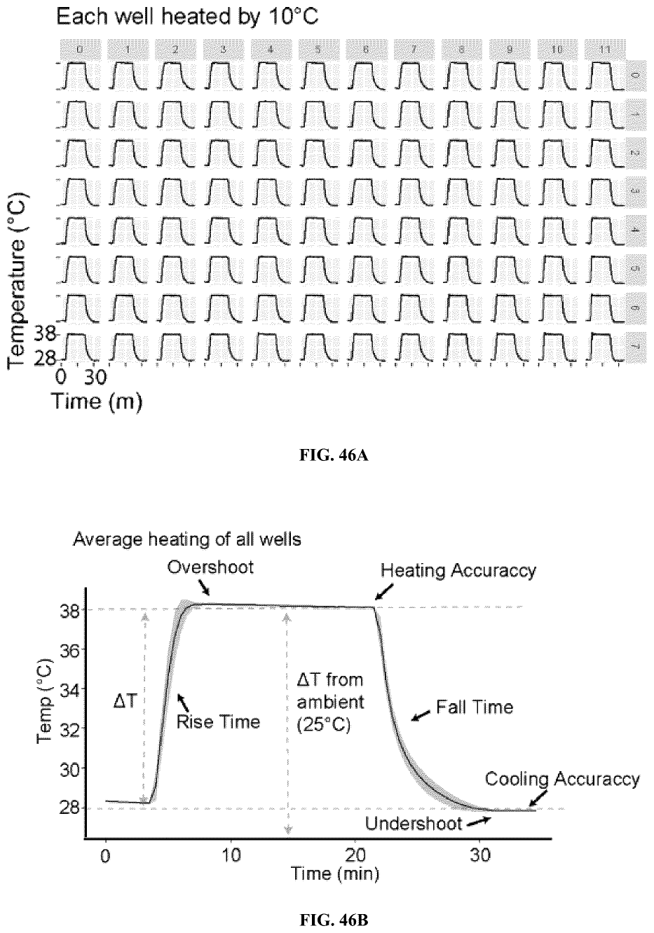

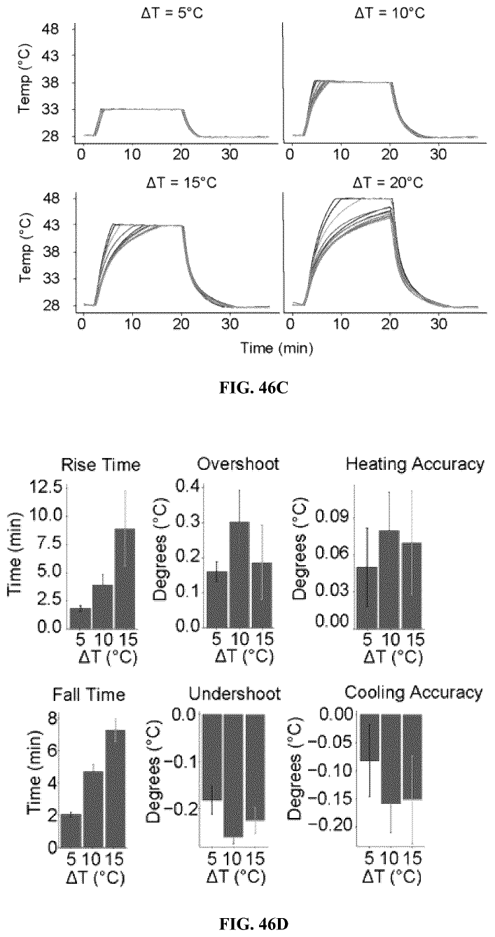

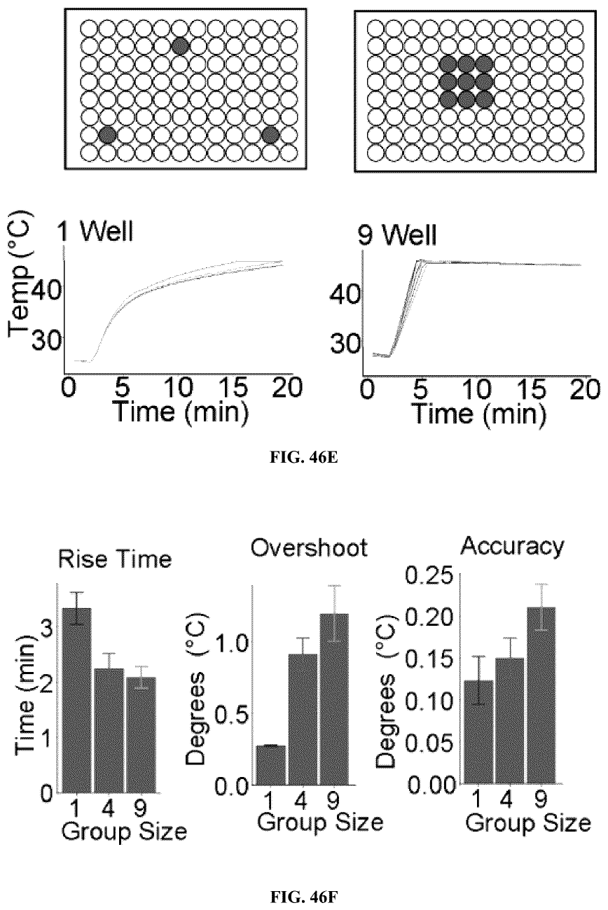

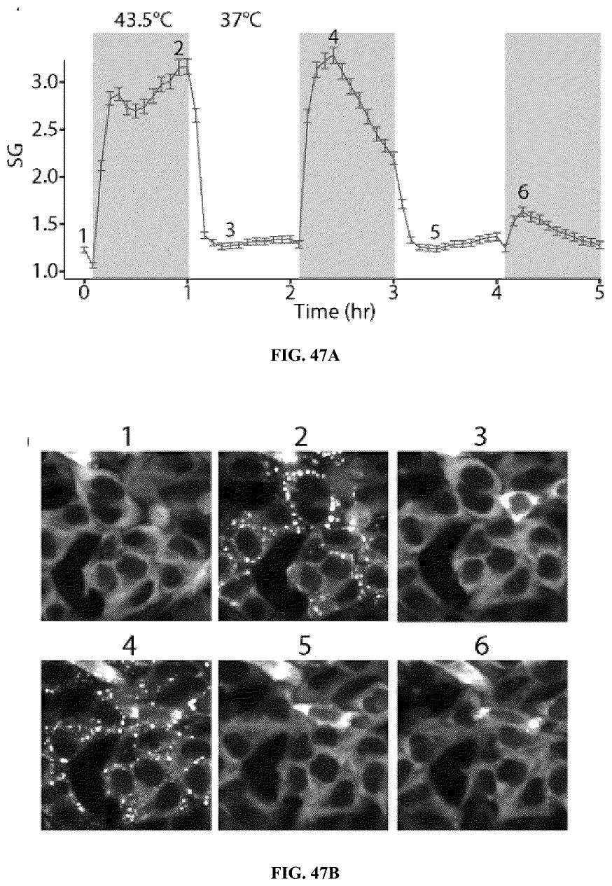

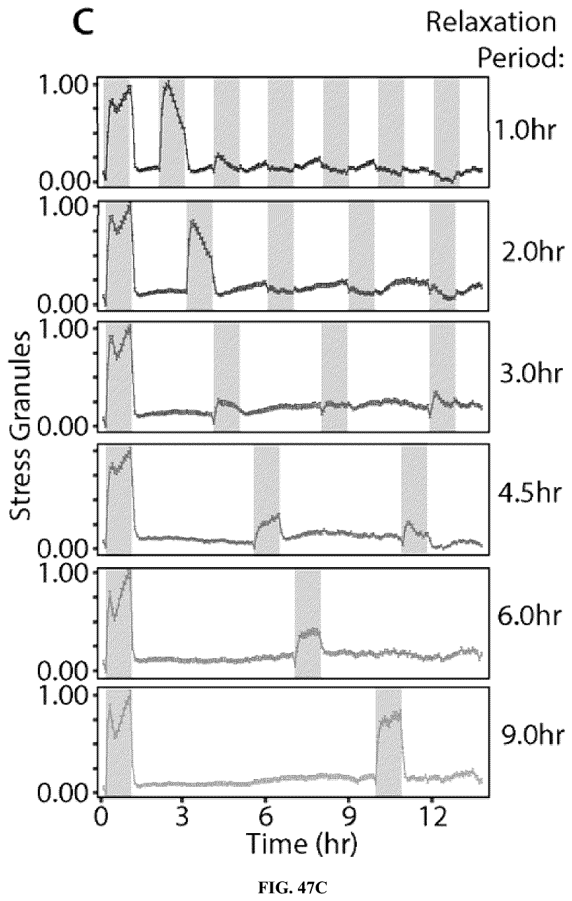

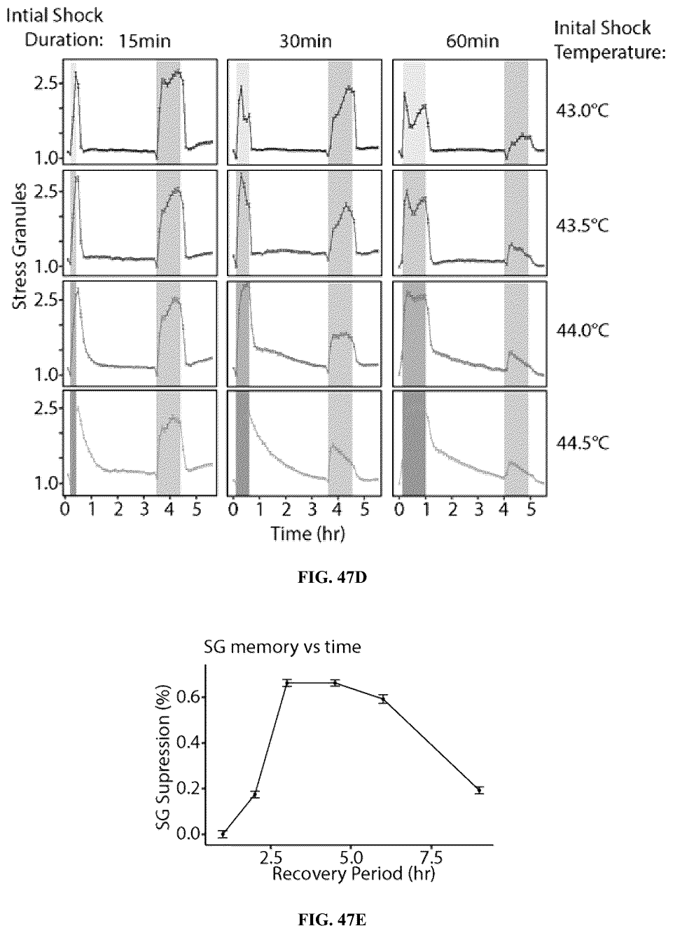

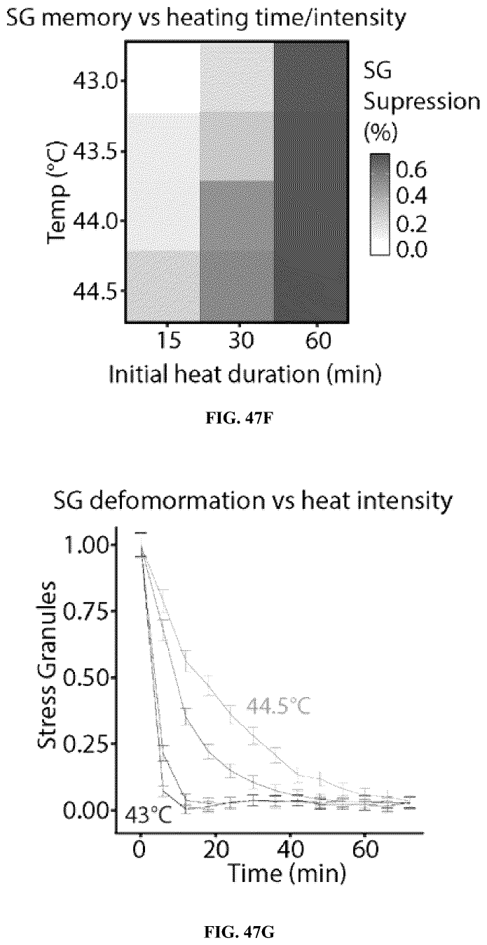

We explored the potential of Melt to control molecular circuits in mammalian cells in response to temperature changes. Recruitment of cargo to/from the membrane is a powerful mode of post-translational control, including for cell signaling.²² We first targeted signaling through the Ras-Erk pathway, a central regulator of cell growth and cancer. We generated an end-to-end fusion of Melt to the catalytic domain of the Ras activator SOS2,²³ an architecture that previously allowed potent stimulation of Ras signaling using optogenetic BcLOV4.¹⁸ We expressed this construct (meltSOS) in HEK 293T cells and measured Erk activation upon changing temperature from 37° C. to 27° C. (FIG. 32A). Active Erk (phospho-Erk, or ppErk) could be observed even within 5 minutes of temperature change to 27° C. and continued to rise until its plateau at 30 mins (FIGS. 32B-C). Conversely, shifting temperature from 27° C. back to 37° C. resulted in measurable signal decrease within 5 min and 172 full decay within 30 mins (FIGS. 32B-C), comparable to the kinetics of thermal inactivation during optogenetic stimulation of BcLOV-SOS.¹⁸

Separately, we tested whether we could leverage the clustering of Melt for control of signaling from the receptor level. We generated a fusion of Melt to the intracellular domain of the epidermal growth factor receptor (EGFR) (FIG. 32D). EGFR is a receptor tyrosine kinase with important roles in development and tumorigenesis and stimulates intracellular signaling through multiple pathways, including Ras-Erk.²⁴ Importantly, both membrane recruitment and clustering of the EGFR intracellular domain are required for its activation.^{20,25} In cells expressing meltEGFR, lowering the temperature from 37° C. to 27° C. activated strong Erk signaling within 10 minutes, and reversion to 37° C. caused signal decay within 5 minutes, with full decay within 30-60 mins (FIGS. 32E-F). Thus, the inducible membrane recruitment and clustering of Melt can be used for rapid, potent, and reversible thermal control of signaling in a modular fashion. When Melt activates proteins at the membrane, it operates as a heat-OFF system. We next examined whether Melt could also implement a heat-ON system by coupling membrane translocation to negative regulation. Proteases can negatively regulate their targets through protein cleavage in both natural and synthetic systems.²⁶⁻²⁸ We thus tested whether Melt could regulate proteolysis at the membrane. We fused Melt to the viral TEV protease (meltTEVp) and we measured whether its membrane recruitment could trigger a membrane-associated reporter of TEVp activity, FlipGFP²⁹ (FlipGFP-CAAX). FlipGFP is non-fluorescent until proteolytic cleavage allows proper folding and maturation of the chromophore (FIG. 32G). Cells that expressed meltTEVp and FlipGFP-CAAX showed minimal levels of fluorescence when cultured at 37° C., similar to cells that expressed FlipGFP-CAAX and cytoplasmic TEVp or FlipGFP-CAAX alone. However, culturing meltTEVp cells at lower temperatures for 24 hours increased FlipGFP fluorescence, with fluorescence increasing monotonically with decreasing temperature, whereas cells expressing cytoplasmic TEVp remained at baseline fluorescence (FIGS. 32H-I, FIGS. 36A-C). Thus, Melt can implement thermal control of proteolysis, providing one method by which it could control downstream circuits as a heat-ON switch.

A second way to convert Melt to heat-ON is to regulate its subcellular compartmentalization. Here, the plasma membrane would sequester Melt, and heat would release sequestration and allow translocation to a separate compartment where it could perform a desired function. As a proof of concept, we engineered Melt to regulate nuclear localization by fusing it to sequences that facilitate nuclear import and export (FIG. 32J). We tested several combinations of nuclear localization sequences (NLS) and nuclear export sequences (NES) to optimize the relative strengths of import and export (FIGS. 37A-D). Melt fused to the SV40 NLS³⁰ and the Stradα NES³¹ showed strong membrane binding and nuclear exclusion at 27° C. and nuclear enrichment when heated to 37° C.(FIGS. 32K-L, SMovie 2). This construct could be dynamically shuttled to and from the nucleus through repeated rounds of heating and cooling. By contrast, Melt without NLS/NES showed no nuclear accumulation upon heating (FIGS. 32K-L). Collectively, our results show that Melt can be applied 215 to control a variety of molecular events, in either heat-ON or heat-OFF configuration, in a straightforward and modular manner.

The utility of Melt in mammals will depend on its ability to induce a strong change in localization in response to temperature, as well as on its ability to operate within a mammalian temperature range (37-42° C.). We thus sought to tune these properties. To increase the magnitude of membrane translocation, we tested whether short polybasic (PB) peptides could strengthen the electrostatic molecular interactions that mediate BcLOV4 membrane binding (FIGS. 33A-B)^{19,32}. We chose two well-characterized PB domains from the STIM1 and Rit proteins, which can enhance membrane-binding of unrelated proteins.³³ End-to-end fusions of Melt to the STIM, tandem STIM (STIM2X), or Rit domains all increased the magnitude of membrane binding at 27° C., in increasing order of strength (FIGS. 33C-D). Kinetic analysis showed that PB domains did not change the rate of Melt dissociation, although some changes in reassociation kinetics were observed (FIGS. 38A-C).

Although PB domains provided a large increase in steady-state membrane binding at 27° C., they provided only a mild increase in thermal switch point to ˜32° C., only 1-2 degrees higher than the original Melt (FIG. 33D). We achieved a more substantial increase through the fortuitous discovery that the C292 residue plays an important role in defining the Melt thermal response. In wt BcLOV4, C292 is thought to form a light-dependent bond with a flavin mononucleotide cofactor that underlies the BcLOV4 photoresponse.¹⁹ Although Melt translocation did not respond to light (FIG. 30G), introduction of a C292A mutation dramatically increased its membrane association not only at 27° C., but also at 37° C. where the original Melt was fully dissociated (FIGS. 33F-G and 39). As before, addition of the STIM PB domain further increased membrane association strength at these higher temperatures. Importantly, both C292A variants retained temperature sensitivity and fully dissociated from the membrane at 41-42° C., with a thermal switch point of 36.5 and 39.5° C. for the C292A and C292A/STIM variants, respectively (FIGS. 33H and 40A-C). Because these Melt variants can exist in one state at 37° C. and another at 41/42° C., they are thus both potentially suitable for actuation within mammalian tissues, with distinct levels of membrane binding and dynamic range that could each be optimal for certain applications. These variants also included a truncation of 96 amino acids from the N-terminal of BcLOV4, which we found expendable, consistent with previous results.¹⁹ Collectively, our work presents four Melt variants with a range of thermal switch points between 30° C. and 40° C., covering temperatures suitable for actuation of a broad range animal cells. We adopted a nomenclature for these variants that reflects these switch-points: Melt-30, Melt-32, Melt-37, and Melt-40.

We tested the ability of the higher switch-point Melt variants to actuate post-translational events between 37 and 42° C. meltEGFR driven by Melt-37 showed strong Erk activation at 37° C. and only baseline levels at 40-41° C (FIGS. 331-J). Erk activity could be stimulated repeatedly over multiple heating/cooling cycles as indicated by the ErkKTR biosensor, which translocates from the nucleus to the cytoplasm upon Erk activation (FIGS. 33K-L, Supplementary Movie 3)³⁴. meltSOS-37 could also stimulate Erk activity but only at <˜ 37° C., potentially reflecting a requirement for higher levels 258 of membrane translocation relative to meltEGFR20 (FIG. 41).

Melt-37/40 could also regulate behaviors that allowed its inversion to a heat-ON signal. Melt-40 fused to TEVp showed strong proteolysis and FlipGFP activation at 37° C., with markedly reduced activity at 41° C. (FIGS. 33M-O). Melt-37 also regulated proteolysis but only induced fluorescence at or below 35° C., and fluorescence fell to near baseline at 37° C. (FIG. 42). These results further highlight that although the general thermal response properties are dictated by the specific Melt variant, the precise thermal switch point of the downstream process can be influenced by the specific fusion partner or the downstream process itself. Melt-40 also regulated membrane-to-nuclear translocation within the well-tolerated 37-41° C. temperature range (FIG. 33P). Fusion to a C-terminal SV40 NLS and Strad « NES allowed strong membrane sequestration at 37° C., and fluorescence became enriched in the nucleus upon heating to 41° C. (FIGS. 33Q-R). As before, translocation was partially reversible on the timescales tested and could be cycled through repeated rounds of heating and cooling (FIGS. 33Q-R, Supplementary Movie 4).

We then asked whether Melt variants could be used to regulate cellular-level behaviors at and above 37° C. We first sought to control cell shape changes through the control of actin polymerization. We fused Melt-37 to the DH-PH domain of Intersectin1 (meltITSN1-37), an activator of the Rho GTPase Cdc42 that has previously been actuated through optogenetic recruitment,³⁵ including with BcLOV4^{36,37} (FIG. 34A). When cooled from 41° C. to 37° C., HEK 293T cells expressing meltITSNI showed rapid and dramatic expansion of lamellipodia and cell size, consistent with Cdc42 activation³⁸ (FIG. 34B). Changes in cell shape could be reversed and re-stimulated over multiple cycles of cooling and heating (FIG. 34C), showing similar magnitude of shape change in each round (FIG. 34D, S9, Supplementary Movie 5). By comparison, temperature changes had no effect on cell shape in cells that expressed Melt-37 without the ITSN1 DH-PH domain.

As a second example, we asked if Melt could be used for thermal control of cell death. Cell death can be achieved by regulated clustering of effector domains of caspase proteins.39 We reasoned that differential clustering of Melt at different temperatures could be leveraged to regulate caspase activity and cell death. We fused Melt-37 to the effector domain of caspase-1 (meltCasp1-37, FIG. 34E), and we measured cell death upon changes in temperature (FIG. 34F). While cells expressing meltCasp1-37 appeared unperturbed at 38° C., transition to 34° C. led to morphological changes within minutes, followed within hours by blebbing and cell death, indicated by both morphology and AnnexinV staining (FIGS. 34G-H, Supplementary Movie 6). ThermoPlate scanning coupled with live cell imaging of AnnexinV allowed us to observe death induction with 1° C. resolution, revealing cell death induction even when shifting temperature by only 1° C. (from 38° C.-37° C.), and the magnitude of cell death increased with larger temperature shifts (FIGS. 341-J). No death was measured in cells expressing Melt-37 without the caspase effector.

Finally, a potential concern for using heat as a stimulus 301 is that heat is a known stressor and could adversely affect cell functions. However, we observed no molecular or functional effects of either the short- or long-term heat profiles used throughout our studies in mammalian cells. Stress granules (SGs), a known consequence of heat-stress,^{40,41} were not observed at 41° C. or below in HEK 293T cells, the operating temperatures for the highest switch-point Melt variants (FIGS. 44A-B). By contrast, SGs could be detected at 42° C. in ˜1-5% of cells, and at 43° C. all cells showed strong SG formation. Of note, existing strategies for thermal induction (e.g. heat shock promoters, thermomers) are typically stimulated with 42° C.,^{4,8,9,17} at the cusp of this non-linear heat-induced SG response (FIG. 44B). We also measured cell proliferation to investigate potential integration of low-level heat stress during multi-hour heating (FIG. 44C). Again, regardless of temperature between 37-42° C, we measured no difference in the fraction of cells with high phospho-Rb levels, a marker of proliferation, or of total cell counts through 24 hr of heating (FIGS. 44D-F).

In sum, membrane binding and clustering of Melt variants can be harnessed to control a diverse array of protein and cell behavior over a broad range of temperatures, including those relevant for mammalian cells, which can be thermally controlled by Melt with a larger buffer from potential heat stress compared to the few alternative approaches.

### DISCUSSION

Here we have described a modular and tunable protein that permits thermal control over a range of molecular and cell-level behaviors. By locking the naturally light- and temperature-sensitive BcLOV4 into its “lit” state, we generated the purely thermoresponsive Melt whose membrane association and clustering can be regulated with a small temperature change (<4° C.). Tuning this thermal response further allowed us to generate multiple variants (Melt-30/32/37/40) whose activation switch points could be shifted within the 30-40° C. range. These variants allowed temperature-inducible control of signaling, proteolysis, and subcellular localization, including between 37° C.-42° C., a critical range for thermal control within mammals. Finally, we showed that Melt can provide thermal control over cell-level behaviors by changing cell size/shape and cell death.

Our engineering efforts provide insight into how the wt BcLOV4 protein senses both light and temperature. Successful isolation of the BcLOV4 thermal response from its light response confirms the distinct molecular nature of these two behaviors, as previously speculated.¹⁸ At the same time, the light and temperature responses are closely linked, since mutation of the C292 residue in the LOV domain, which mediates photo-responsiveness, dramatically shifted the thermal switchpoint of Melt (FIG. 33E). Further mechanistic and structural work will be required to fully understand the molecular basis for BcLOV thermal sensitivity, potentially allowing optimization of Melt properties including speed of response and degree of reversibility, and will shed light on how the photosensing and thermosensing elements of BcLOV4 interact. These latter studies will additionally provide insight for how to engineer novel multi-input proteins that can perform complex logic in response to user-defined stimuli.

Our work also introduces the thermoPlate, a device for independent reading 344 and writing of temperature within each well of a 96-well plate. The thermoPlate allows rapid (˜minutes) and dynamic heating and cooling of samples, which allowed quantitative systematic characterization of the kinetics and reversibility of multiple Melt variants. Importantly, multiplexed control of temperature with the thermoPlate is constrained by thermal diffusion, since a hot well will influence the temperature in neighboring wells. However, with careful definition of sample position within a plate, choice of ambient temperature, and PID feedback control, the challenges of thermal diffusion can be overcome. The thermoPlate is fully open source and can be assembled in under 6 hours for ˜$400. We anticipate this device will be highly enabling for any use case where multiplexed or dynamic thermal control is required.

Multiplexed control of sample temperature allowed us to systematically characterize new Melt variants, ultimately resulting in variants with switch-points ranging from 30-40° C. Because BcLOV4 works in mammalian cells but also in systems that are cultured at lower temperatures like yeast, flies, zebrafish, and ciona, 18,19,36,42-44, we anticipate that all Melt variants will find use across these and similar settings. Our work also highlights the utility of having multiple variants in hand to optimize specific downstream applications. We found on multiple occasions that the precise thermal response profiles depended not only on the specific Melt variant but also on the downstream process under control, requiring empirical validation for each use case and biological context. Optimization can be performed by testing other Melt variants, or by generating new ones through additional modifications (e.g. polybasic domains) or mutations. Melt dramatically expands the range of molecular and cellular events that can be controlled by temperature, and in mammalian cells allows thermal control with lower potential for heat stress relative to the few existing approaches. Melt provides an orthogonal input control on biological systems that can be used in conjunction with-or instead of-existing technologies based on light or chemicals, promising to expand the sophistication and reach of biological control with broad potential for biotechnology and biomedicine.

### METHODS

### Cell Culture

Lenti-X HEK 293T cells were maintained in 10% fetal bovine serum (FBS) and 1% penicillin/streptomycin (P/S) in DMEM. (Lenti-X HEK 293T: Takarabio 632180). Cell lines were not verified after purchase. Cells were not cultured in proximity to commonly misidentified cell lines.

### Plasmid Design and Assembly

Constructs for stable transduction and transient transfection were cloned into the pHR lentiviral backbone with a CMV promoter driving the gene of interest. Melt mutations were introduced to WT BcLOV4 (Provided by Brian Chow) (Addgene Plasmid #114595) via whole backbone PCR using primers containing the target mutation. Mutations were introduced using the same primers on BcLOV4-ITSNI (Provided by Brian Chow) (Addgene #174509) to generate meltITSN1-37. Melt-PB fusions were generated via whole backbone PCR using primers containing PB coding sequences (FIG. 31B). PCR products were circularized via ligation (New England Biolabs). For Melt-effector fusions, the pHR backbone was linearized using MluI and NotI restriction sites. Melt, TEVp (Addgene Plasmid #8827), EGFR (sourced from Opto-hEGFR), SOS,¹⁸ and Caspase-1³⁹ were generated via PCR and inserted into the pHR backbone via HiFi cloning mix (New England Biolabs). All Melt37/40-Effector fusions were generated by amplifying Melt37/40 with primers that amplified the region downstream of a.a.96 such that the final Melt variants contained a a.a. 1-96 deletion. NLS/NES insertions were generated via backbone PCRs with NLS/NES sequences (FIGS. 37A-D) incorporated into the primers. To construct FlipGFP-BFP-CAAX, the two fragments of FlipGFP B1-9 and B10-E5-B11-TEVcs-K5 were amplified from Addgene Plasmid #124429 via PCR. tagBFP¹⁸ was amplified using primers containing a CAAX membrane binding sequence. These fragments were assembled in the linearized PHR backbone via HiFi cloning mix in the order B1-9-P2A-B10-E5-B11-TEVcs-K5-tagBFP-CAAX. In order to reduce affinity of TEVp for the TEV cut site (cs) and lower basal proteolysis, the canonical cut site ENLYFQS was mutated to ENLYFQL⁴⁵ via whole backbone PCR using primers harboring the mutation. GFP-CAAX was generated via PCR of eGFP using primers containing the CAAX sequence and cloned into the linearized viral backbone using HiFi cloning mix.

### Plasmid Transfection.

HEK 293T cells were transfected using the calcium phosphate method, as follows: Per 1 mL of media of the cell culture to be transfected, 50 μL of 2×HeBS^{28,29} buffer, 1 μg of each DNA construct, and H₂O up to 94 μL was mixed. 6 μL of 2.5mM CaCl₂ was added after mixing of initial components, incubated for 1:45 minutes at room temperature, and added directly to cell culture.

### Lentiviral Packaging and Cell Line Generation

Lentivirus was packaged by cotransfecting the pHR transfer vector, pCMV-dR8.91 (Addgene, catalog number 12263), and pMD2.G (Addgene, catalog number 12259) into Lenti-X HEK293T. Briefly, cells were seeded one day prior to transfection at a concentration of 350,000 cells/mL in a 6-well plate. Plasmids were transfected using the calcium phosphate 706 method. Media was removed one day post-transfection and replaced with fresh media. Two days post-transfection, media containing virus was collected and centrifuged at 800×g for 3 minutes. The supernatant was passed through a 0.45 μm filter. 500 μL of filtered virus solution was added to 700,000 HEK293T cells seeded in a 6-well plate. Cells were expanded over multiple passages, and successfully transduced cells were enriched through fluorescence activated cell sorting (Aria Fusion).

### Preparation of Cells for Plate-Based Experiments

All experiments were carried out in Cellvis 96 well plates (#P96-1.5P). Briefly, wells were coated with 50 uL of MilliporeSigma™ Chemicon™ Human Plasma Fibronectin Purified Protein fibronectin solution diluted 100×in PBS and were incubated at 37° C. for 30 min. HEK 293T cells were seeded in wells at a density of 35,000 cells/well in 100 u L and were spun down at 20×g for 1 minute. In experiments requiring starvation (for all experiments involving SOS and EGFR constructs), after 24 hr, cells were starved by performing 7 80% washes with starvation media (DMEM+1% P/S). Experiments were performed after 3 hr of starvation.

### Fixing and Immunofluorescence Staining

Immediately following the completion of a temperature stimulation protocol, 16% paraformaldehyde (PFA) was added to each well to a final concentration of 4%, and cells were incubated in PFA for 10 min. For immunofluorescence staining, cells were then permeabilized with 100 μL phosphate buffered saline (PBS)+0.1% Triton-X for 10 min. Cells were then further permeabilized with ice cold methanol for 10 min. After permeabilization, cells were blocked with 1% BSA at room temperature for 30 min. Primary antibody was diluted in PBS+1% BSA according to the manufacturer's recommendation for immunofluorescence (phospho-p44/42 MAPK (Erk1/2) (Thr202/Tyr204), Cell Signaling #4370, 1:400 dilution; phospho-Rb (Ser807/811) Cell Signaling #9308, 1:800 dilution; Anti-Human G3BP1, BD Biosciences #611126, 1:500 dilution). Wells were incubated with 50 μL of antibody dilution for 2 hr at room temperature (RT), after which primary antibody was removed and samples underwent five washes in PBS+0.1% TWEEN-20 (PBS-T). Cells were then incubated with secondary antibody (Jackson Immunoresearch Alexa FluorR 488 AffiniPure Goat Anti-Rabbit IgG (H+L) or Invitrogen Goat anti-Mouse IgG (H+L) Cross-Adsorbed Secondary Antibody, DyLight™ 650) and DAPI (ThermoFisher, #D1306, 300 nM) in PBS-T+0.1% BSA for 1 hour at RT. Secondary antibody was removed, samples underwent 5 washes with PBS-T. Samples were imaged in PBS-T.

### Imaging

Live-cell imaging. Live-cell imaging was performed using a Nikon Ti2-E microscope equipped with a Yokagawa CSU-W1 spinning disk, 405/488/561/640 nm laser lines, an sCMOS camera (Photometrics), a motorized stage, and an environmental chamber (Okolabs). HEK 293Ts expressing the construct of interest were imaged with 744 a 20× or 40× objective at variable temperatures and 5% CO2. Optogenetic BcLOV4 was stimulated using a 488 nm laser.

High content fixed-cell imaging. Fixed samples were imaged using a Nikon Ti2E epifluorescence microscope equipped with DAPI/FITC/Texas Red/Cy5 filter cubes, a SOLA SEII 365 LED light source, and motorized stage. High content imaging was performed using the Nikon Elements AR software. Image focus was ensured using image-based focusing in the DAPI channel.

### Image Processing and Analysis

Immunofluorescence quantification. Images were processed using Cell Profiler. Cells were segmented using the DAPI channel, and cytoplasm was identified using a 5 pixel ring around the nucleus. Nuclear and cytoplasmic fluorescence values were then exported and analyzed using R (https://cran.r-project.org/) and R-Studio (https://rstudio.com/). Data was processed and visualized using the tidyR⁴⁶ and ggplot2⁴⁷ packages.

Membrane recruitment. Membrane localization was quantified using the MorphoLibJ plugin for ImageJ⁴⁸. Briefly, MorphoLibJ was used to segment single cells based on a constitutively membrane bound GFP-CAAX marker. The resulting segmentation was imported into Cell Profiler and was used to quantify the amount of mCherry (fused to the protein of interest) localized to the membrane as well as total mCh per cell (FIG. 35). Total mCh and membrane-localized mCh intensity was recorded and further processed in R. Bleaching was corrected by dividing the membrane intensity of mCh by total cell mCh.

FlipGEP Quantification. Cells expressing membrane bound FlipGFP-CAAX and the indicated TEVp construct were grown at the indicated temperature and fixed in 4% PFA after 24 hours. FlipGFP was tethered to the membrane via a Blue Fluorescent Protein (TagBFP)-CAAX fusion. BFP-CAAX remained tethered to the membrane before and after proteolysis and thus could be used as a membrane marker. This marker was used to segment single cells using the same workflow used for membrane recruitment quantification. Single cell GFP levels were quantified using Cell Profiler and used as an indicator of relative levels of proteolysis.

Nuclear Localization. To quantify nuclear localization of a protein of interest, cells expressing a GFP-CAAX membrane marker (see above) were transfected with an H2B-iRFP nuclear marker. The above workflow was used to segment individual cells based on the membrane marker. This segmentation was imported to CellProfiler, which was also used to segment nuclei based on iRFP imaging. Each nucleus was then assigned to a parent cell. Nuclei were assigned to a cell if >90% of the nucleus object was contained by the cell object. Membrane segmented cells that contained no nuclei objects or nuclei that were not within a parent cell were eliminated from quantification. Finally, nuclear to total cell mCherry (used as a marker fused to the protein of interest) was calculated and recorded for each cell. Annexin Staining and Quantification. Annexin V-647 (Invitrogen A23204) was added to 100 uL of cell culture at a 1:100 final dilution. A final concentration of 1 mM CaCl2 was also added to each well to allow Annexin V cell labeling. Cell media was removed and replaced with Annexin V media 30 min prior to imaging. To quantify Annexin V, images of cells 783 expressing meltCasp1-37 or Melt-37 both with a GFP fusion were used to create GFP masks using CellProfiler's threshold function. Annexin images were masked for GFP positive pixels. The total masked Annexin image intensity was recorded and normalized by the number of GFP positive pixels (cell area per image) in each image.

Cell Area Quantification. Cell area was measured semi-manually. Images of cells expressing meltITSN1-37 and Melt-37 were imaged and resulting images were thresholded in ImageJ such that cell positive pixels were set to 1 and background pixels were set to 0. Cells were manually chosen for quantification and regions containing the cell of interest were drawn by hand. Measuring integrated pixel intensity of these regions gave rise to the number of cell positive pixels in that region which was used as a metric of total cell area. For further explanation, see FIG. 43.

### Curve Fitting

Data points for Melt variant equilibrium membrane binding at various temperatures were fit to the Hill Equation (Eq.1). MATLAB was used to minimize the error between the sigmoid function and each data point. The characteristic function used for fitting was:

F(x)=A*x^{B}/(C^{B}+x^{B}) (1)

A, B, and C were used as the adjusted parameters. These curves are displayed in FIGS. 31E, 33D, and 33H with datapoints overlaid. The associated code can be found in this manuscript's code repository (https://rb.gy/1k7tc).

### Example 4

### Overview of ThermoPlate

FIGS. 45A-47G demonstrate the design and use of the thermoPlate device according to one or more embodiments disclosed herein. More specifically, FIGS. 45A-H illustrate the design and use of the thermoPlate for simultaneous, independent temperature control of multiple wells in a 96 well plate. FIGS. 46A-F illustrate characterization of thermoPlate performance during heating and cooling. FIGS. 47A-G illustrate that the thermoPlate allows interrogation of stress granule dynamics in a high-throughput format.

While this invention has been disclosed with reference to specific embodiments, it is apparent that other embodiments and variations of this invention may be devised by others 10 skilled in the art without departing from the true spirit and scope of the invention. The appended claims are intended to be construed to include all such embodiments and equivalent variations.

What is claimed is:

1. A device for well plate temperature control, the device comprising:
a microwell plate comprising:
a top well plate surface;
a bottom well plate surface; and
at least one well extending from the top well plate surface towards the bottom well plate surface;
a temperature control assembly comprising:
a printed circuit board having:
a top circuit board surface; and
a bottom circuit board surface; and
at least one pair of thermistors extending outwardly from the bottom circuit board surface, each of the at least one pair of thermistors arranged and disposed to align with one of the at least one wells when the bottom circuit board surface is positioned adjacent to the top well plate surface; and
a microcontroller configured to individually control each of the at least one pair of thermistors.

2. The device according to claim 1, wherein each pair of thermistors comprises a heating thermistor and a measurement thermistor.

3. The device according to claim 1, wherein the heating thermistor comprises a resistive heating thermistor.

4. The device according to claim 1, wherein the temperature control assembly further comprises at least one shift register and at least one control transistor.

5. The device according to claim 4, wherein the microcontroller communicates with the at least one shift register to control the at least one control transistor, and the at least one control transistor determines the current flow through each of the thermistors.

6. The device according to claim 1, wherein the temperature control assembly further comprises at least one voltage divider and at least one multiplexer in electrical communication with the microcontroller.

7. The device according to claim 6, wherein the temperature of each of the at least one wells is measured through the at least one voltage divider and the at least one multiplexer.

8. The device according to claim 1, wherein the at least one well comprises 96 wells.

9. The device according to claim 1, wherein each of the at least one pair of resistors includes a liquid-tight covering.

10. The device according to claim 9, wherein the liquid-tight covering comprises heatshrink tubing.

11. The device according to claim 10, wherein the liquid-tight covering further comprises a conformal coating over the heatshrink tubing.

12. The device according to claim 11, wherein the conformal coating comprises a silicone conformal coating.

13. The device according to claim 9, wherein each of the resistors independently includes the liquid-tight covering.

14. The device according to claim 1, further comprising an individual feedback loop between the microcontroller and each of the at least one pair of thermistors.

15. The device according to claim 1, further comprising:
an adapter positioned between the microwell plate and the temperature control assembly;
wherein the adapter is arranged and disposed to position the temperature control assembly relative to the well plate.

16. A method of independently controlling the temperature in individual wells of a microwell plate, the method comprising:
providing the device according to claim 1;
filling one or more of the at least one wells with a liquid sample;
positioning the bottom circuit board surface adjacent to the top well plate surface, the positioning immersing the at least one pair of thermistors in the one or more wells with the liquid sample;
independently providing a current flow to each of the at least one pair of thermistors, the current flow to each of the at least one pair of thermistors providing a desired temperature in the corresponding well through resistive heating.

17. The method of claim 16, wherein the device further comprises an adapter positioned between the microwell plate and the temperature control assembly, the adapter being arranged and disposed to position the temperature control assembly relative to the well plate.

18. A temperature-responsive protein comprising:
a BcLOV4 protein variant having a point mutation at Q355;
wherein the variant includes at least 80% sequence homology with the wild-type BcLOV4 protein.

19. The protein according to claim 18, wherein the Q355 point mutation is Q355N.

20. The protein according to claim 18, further comprising a point mutation at C292.

21. The protein according to claim 20, wherein the C292 point mutation is selected from the group consisting of C292A, C292R, C292N, C292D, C292E, C292Q, C292G, C292H, C292I, C292L, C292K, C292M, C292F, C292P, C292S, C292T, C292W, C292Y, C292V.

22. The protein according to claim 20, wherein the C292 point mutation is C292A.

23. The protein according to claim 20, wherein the C292 point mutation is selected from the group consisting of C292R, C292N, C292D, C292E, C292Q, C292G, C292H, C292I, C292L, C292K, C292M, C292F, C292P, C292S, C292T, C292W, C292Y, C292V.

24. The protein according to claim 18, wherein the point mutation further comprises deletion of one or more of amino acids 1-97.

25. A method of controlling the membrane localization of a protein, the method comprising:
providing the protein according to claim 18; and
exposing the protein to a temperature above or below an activation temperature.

26. The method according to claim 25, wherein the C292 point mutation is selected from the group consisting of C292A, C292R, C292N, C292D, C292E, C292Q, C292G, C292H, C292I, C292L, C292K, C292M, C292F, C292P, C292S, C292T, C292W, C292Y, C292V.
